SEQUENCE LISTING (1926 sequences)

SEQ ID NO: 1 - DNA, 2225 bp, Homo sapiens
agcgggactggagaggagtgagtggcgggctgggcggcggcggccgtagccgcgggtgcctccccgcctcaccgcttcgcaggcagcaccgcctcccgggccgcggccgcccggccgggtcctctgcgcgttcccgggcccggaaccggcacacgatgcacccggctgcgttctgatcgccgccgcgcctcagcctctgccgcgatgatcccccctgagcagccgcagcagcagctgcagccgccgtcgccagccccgccgaaccatgtggtgaccaccatcgagaacctgccggccgagggcagcggcggcggcgggagcctgtccgcctcctcccgggctggcgtgcgccagaggatccgcaaagtgctgaacagggagatgttaatctctgtggccctaggccaggtgttatccctccttatttgtggaattggcttgactagcaagtatctgtcagaagatttccacgccaacacaccagtcttccagagtttcctcaattacattcttctcttcttggtctataccaccacactagccgtcagacaaggagaagaaaacctcctggcaattttacgacgaagatggtggaagtacatgattttgggactcatagacctggaagcaaattatctggtggtcaaggcttaccaatacacaactctgaccagtatccagctcctggactgttttgtgatcccagtcgtgattttgctctcctggttcttcctgctgatccggtacaaggctgtgcatttcatcggcatcgttgtctgcatcctgggaatgggctgcatggtgggagcagatgtgcttgtgggaagacatcagggagcaggggaaaataagctggtaggggaccttctggtcttaggaggagccacactctatggtatttctaacgtctgggaagaatacatcatccgaactctgagccgagtggaattcctgggaatgattggtctctttggagcatttttcagtggaattcaattagctataatggagcataaggaactgttgaaggtgccctgggactggcaaataggactgctctacgttggctttagtgcctgcatgtttggtctctacagctttatgccagtcgtcataaagaaaaccagtgccacttcagtcaacctctccttgctcacagcagacttgtacagcctgttctgtggattgtttctcttccactacaagttttcaggactttatctcctgtctttcttcaccatcctcattgggctggtgctctactcctccacctccacctacatagcccaggacccccgagtgtataagcagttccgcaatccttcaggacctgttgtggacttaccgaccacagctcaggtggaaccctcagtcacctacaccagcctgggccaggagaccgaagaggagcctcatgttcgtgtggcctagggtgaggcccgccctgccaactgaggccaactcattggccatgtttttgcccatcatctctgtattgtacatagagaaaggtatttactaggtgcagtttacacaggtggactgcaaggtagcaaatcctccaaaagcttgtgaaaggaacaagctcaacatcactggagacacaggctctaatccacctgacttggaaggatgcctagctaacgtgtatcctgatcacaactcccctgcattcattactgtgaaaatttttgaatcaaaagcaagtattattgttattatgatttgtattattattattgctgttactgttattacaccaactttcaggaggatgttttgtactcctgatggaaactattgccagacccacatgtagtcaacataaaccccacttttctatggcaattcactttttaagagtcatactttatttttttgcacagactatatgagtgatgcatgccacaggagctccacccctgagaagtttccttttcctagggacctggtggttaatggtttcctctgttacctgttggattgcctgctcaataagtattttgtgctgtgacctttgtggggaggggcaactgaccatatacttctctattctaggaatagatataaaagaaacattttagcctttttatatttcgatctctgattactccaggccattgcctttctgttgtgccacgtgattctgctaatcaagtccctgtaataacaggataaggactgttccattactgtggcttttgggagcccccagaaataatatc

SEQ ID NO: 2 - DNA, 1227 bp, Homo sapiens
atgatcccccctgagcagccgcagcagcagctgcagccgccgtcgccagccccgccgaaccatgtggtgaccaccatcgagaacctgccggccgagggcagcggcggcggcgggagcctgtccgcctcctcccgggctggcgtgcgccagaggatccgcaaagtgctgaacagggagatgttaatctctgtggccctaggccaggtgttatccctccttatttgtggaattggcttgactagcaagtatctgtcagaagatttccacgccaacacaccagtcttccagagtttcctcaattacattcttctcttcttggtctataccaccacactagccgtcagacaaggagaagaaaacctcctggcaattttacgacgaagatggtggaagtacatgattttgggactcatagacctggaagcaaattatctggtggtcaaggcttaccaatacacaactctgaccagtatccagctcctggactgttttgtgatcccagtcgtgattttgctctcctggttcttcctgctgatccggtacaaggctgtgcatttcatcggcatcgttgtctgcatcctgggaatgggctgcatggtgggagcagatgtgcttgtgggaagacatcagggagcaggggaaaataagctggtaggggaccttctggtcttaggaggagccacactctatggtatttctaacgtctgggaagaatacatcatccgaactctgagccgagtggaattcctgggaatgattggtctctttggagcatttttcagtggaattcaattagctataatggagcataaggaactgttgaaggtgccctgggactggcaaataggactgctctacgttggctttagtgcctgcatgtttggtctctacagctttatgccagtcgtcataaagaaaaccagtgccacttcagtcaacctctccttgctcacagcagacttgtacagcctgttctgtggattgtttctcttccactacaagttttcaggactttatctcctgtctttcttcaccatcctcattgggctggtgctctactcctccacctccacctacatagcccaggacccccgagtgtataagcagttccgcaatccttcaggacctgttgtggacttaccgaccacagctcaggtggaaccctcagtcacctacaccagcctgggccaggagaccgaagaggagcctcatgttcgtgtggcctag

SEQ ID NO: 3 - protein, 408 aa, Homo sapiens
MIPPEQPQQQLQPPSPAPPNHVVTTIENLPAEGSGGGGSLSASSRAGVRQRIRKVLNREMLISVALGQVLSLLICGIGLTSKYLSEDFHANTPVFQSFLNYILLFLVYTTTLAVRQGEENLLAILRRRWWKYMILGLIDLEANYLVVKAYQYTTLTSIQLLDCFVIPVVILLSWFFLLIRYKAVHFIGIVVCILGMGCMVGADVLVGRHQGAGENKLVGDLLVLGGATLYGISNVWEEYIIRTLSRVEFLGMIGLFGAFFSGIQLAIMEHKELLKVPWDWQIGLLYVGFSACMFGLYSFMPVVIKKTSATSVNLSLLTADLYSLFCGLFLFHYKFSGLYLLSFFTILIGLVLYSSTSTYIAQDPRVYKQFRNPSGPVVDLPTTAQVEPSVTYTSLGQETEEEPHVRVA

SEQ ID NO: 4 - DNA, 53 bp, Homo sapiens
ccgcccggccgggtcctctgcgcgttcccgggcccggaaccggcacacgatgc

SEQ ID NO: 5 - DNA, 427 bp, Homo sapiens
ttcctgctgatccggtacaaggctgtgcatttcatcggcatcgttgtctgcatcctgggaatgggctgcatggtgggagcagatgtgcttgtgggaagacatcagggagcaggggaaaataagctggtaggggaccttctggtcttaggaggagccacactctatggtatttctaacgtctgggaagaatacatcatccgaactctgagccgagtggaattcctgggaatgattggtctctttggagcatttttcagtggaattcaattagctataatggagcataaggaactgttgaaggtgccctgggactggcaaataggactgctctacgttggctttagtgcctgcatgtttggtctctacagctttatgccagtcgtcataaagaaaaccagtgccacttcagtcaacctctccttgctca

SEQ ID NO: 6 - protein, 142 aa, Homo sapiens
FLLIRYKAVHFIGIVVCILGMGCMVGADVLVGRHQGAGENKLVGDLLVLGGATLYGISNVWEEYIIRTLSRVEFLGMIGLFGAFFSGIQLAIMEHKELLKVPWDWQIGLLYVGFSACMFGLYSFMPVVIKKTSATSVNLSLL

SEQ ID NO: 7 - DNA, 524 bp, Homo sapiens
tgtgaaaggaacaagctcaacatcactggagacacaggctctaatccacctgacttggaaggatgcctagctaacgtgtatcctgatcacaactcccctgcattcattactgtgaaaatttttgaatcaaaagcaagtattattgttattatgatttgtattattattattgctgttactgttattacaccaactttcaggaggatgttttgtactcctgatggaaactattgccagacccacatgtagtcaacataaaccccacttttctatggcaattcactttttaagagtcatactttatttttttgcacagactatatgagtgatgcatgccacaggagctccacccctgagaagtttccttttcctagggacctggtggttaatggtttcctctgttacctgttggattgcctgctcaataagtattttgtgctgtgacctttgtggggaggggcaactgaccatatacttctctattctaggaatagatataaaagaaacattttagcctttttata

SEQ ID NO: 8 - DNA, 374 bp, Homo sapiens
gggactggagaggagtgagtggcgggctgggcggcggcggccgtagccgcgggtgcctccccgcctcaccgcttcgcaggcagcaccgcctcccgggccgcggccgcccggccgggtcctctgcgcgttcccgggcccggaaccggcacacgatgcacccggctgcgttctgatcgccgccgcgcctcagcctctgccgcgatgatcccccctgagcagccgcagcagcagctgcagccgccgtcgccagccccgccgaaccatgtggtgaccaccatcgagaacctgccggccgagggcagcggcggcggcgggagcctgtccgcctcctcccgggctggcgtgcgccagaggatccgcaaagtgctgaacag

SEQ ID NO: 9 - DNA, 176 bp, Homo sapiens
ggagatgttaatctctgtggccctaggccaggtgttatccctccttatttgtggaattggcttgactagcaagtatctgtcagaagatttccacgccaacacaccagtcttccagagtttcctcaattacattcttctcttcttggtctataccaccacactagccgtcagacaag

SEQ ID NO: 10 - DNA, 128 bp, Homo sapiens
gagaagaaaacctcctggcaattttacgacgaagatggtggaagtacatgattttgggactcatagacctggaagcaaattatctggtggtcaaggcttaccaatacacaactctgaccagtatccag

SEQ ID NO: 11 - DNA, 160 bp, Homo sapiens
ctcctggactgttttgtgatcccagtcgtgattttgctctcctggttcttcctgctgatccggtacaaggctgtgcatttcatcggcatcgttgtctgcatcctgggaatgggctgcatggtgggagcagatgtgcttgtgggaagacatcagggagcag

SEQ ID NO: 12 - DNA, 157 bp, Homo sapiens
gggaaaataagctggtaggggaccttctggtcttaggaggagccacactctatggtatttctaacgtctgggaagaatacatcatccgaactctgagccgagtggaattcctgggaatgattggtctctttggagcatttttcagtggaattcaatt

SEQ ID NO: 13 - DNA, 53 bp, Homo sapiens
agctataatggagcataaggaactgttgaaggtgccctgggactggcaaatag

SEQ ID NO: 14 - DNA, 155 bp, Homo sapiens
gactgctctacgttggctttagtgcctgcatgtttggtctctacagctttatgccagtcgtcataaagaaaaccagtgccacttcagtcaacctctccttgctcacagcagacttgtacagcctgttctgtggattgtttctcttccactacaag

SEQ ID NO: 15 - DNA, 1019 bp, Homo sapiens
ttttcaggactttatctcctgtctttcttcaccatcctcattgggctggtgctctactcctccacctccacctacatagcccaggacccccgagtgtataagcagttccgcaatccttcaggacctgttgtggacttaccgaccacagctcaggtggaaccctcagtcacctacaccagcctgggccaggagaccgaagaggagcctcatgttcgtgtggcctagggtgaggcccgccctgccaactgaggccaactcattggccatgtttttgcccatcatctctgtattgtacatagagaaaggtatttactaggtgcagtttacacaggtggactgcaaggtagcaaatcctccaaaagcttgtgaaaggaacaagctcaacatcactggagacacaggctctaatccacctgacttggaaggatgcctagctaacgtgtatcctgatcacaactcccctgcattcattactgtgaaaatttttgaatcaaaagcaagtattattgttattatgatttgtattattattattgctgttactgttattacaccaactttcaggaggatgttttgtactcctgatggaaactattgccagacccacatgtagtcaacataaaccccacttttctatggcaattcactttttaagagtcatactttatttttttgcacagactatatgagtgatgcatgccacaggagctccacccctgagaagtttccttttcctagggacctggtggttaatggtttcctctgttacctgttggattgcctgctcaataagtattttgtgctgtgacctttgtggggaggggcaactgaccatatacttctctattctaggaatagatataaaagaaacattttagcctttttatatttcgatctctgattactccaggccattgcctttctgttgtgccacgtgattctgctaatcaagtccctgtaataacaggataaggactgttccattactgtggcttttgggagcccccagaaataatatc

SEQ ID NO: 16 - DNA, 500 bp, Homo sapiens
gacggaggggacccggagccgggcgcccgcagcccccgccccccgcgccctcgcccggcgccgccgctcacctgttcagcactttgcggatcctctggcgcacgccagcccgggaggaggcggacaggctcccgccgccgccgctgccctcggccggcaggttctcgatggtggtcaccacatggttcggcggggctggcgacggcggctgcagctgctgctgcggctgctcaggggggatcatcgcggcagaggctgaggcgcggcggcgatcagaacgcagccgggtgcatcgtgtgccggttccgggcccgggaacgcgcagaggacccggccgggcggccgcggcccgggaggcggtgctgcctgcgaagcggtgaggcggggaggcacccgcggctacggccgccgccgcccagcccgccactcactcctctccagtcccagtgcgcgcaccctcgcgcccgtgccgccgccgccgccgcccgccccggctcttc

SEQ ID NO: 17 - DNA, 500 bp, Homo sapiens
tcattgcaccttcctcgagggattgctttgatggactaaagggaataatatttaattgcacttaatccagtgcatgctaccagtgctcaaaaagttttaattgctattgttttaatgggcttcctgctgacctttgctgtgttttttttcctttatttctcagggagatgttaatctctgtggccctaggccaggtgttatccctccttatttgtggaattggcttgactagcaagtatctgtcagaagatttccacgccaacacaccagtcttccagagtttcctcaattacattcttctcttcttggtctataccaccacactagccgtcagacaaggtaagctcacaaaagcaccaggaatataacttttacaaacacctaaaaaaaagatgagccatgaccagataacgttttgtcacttaagcatcagttaagatcattgaaaaccccagatcttacttttctattttgcagtaatacagtaaacaaaatgagtg

SEQ ID NO: 18 - DNA, 500 bp, Homo sapiens
atctgcaataaagttaatttgctaaataaaaacatgtgggtatctgatacgttgatttatttgtttaaatatttggtaaactttaggttaaaaatatataatgttgcaacatattagaattcttctaaatgtacaatttattctatttcaggaacctaacccatttcttcttaactttgtaacacaggagaagaaaacctcctggcaattttacgacgaagatggtggaagtacatgattttgggactcatagacctggaagcaaattatctggtggtcaaggcttaccaatacacaactctgaccagtatccaggtacccatggttcttcttcccttctcaacttcctctatccagatgtagtttactttcagtttagtccttgaaaatctctatactacaagttactatctgtatactatactatagtatacagactgcagtatacagattctgtatactataagtctatgagttttgcaacaagatggaaactggtt

SEQ ID NO: 19 - DNA, 500 bp, Homo sapiens
ccatttatttcagagtctgttttaagtacatcaagtctgggtctacacaaaatctcaggttcaggggaaaggcagaaccctttctgcagaactaaattacttttccctactacatggaatgcttcctcctgaatctcctgttgttgtttaccttcatccctcccaatagctcctggactgttttgtgatcccagtcgtgattttgctctcctggttcttcctgctgatccggtacaaggctgtgcatttcatcggcatcgttgtctgcatcctgggaatgggctgcatggtgggagcagatgtgcttgtgggaagacatcagggagcaggtgagtcttcggatgttcaccaggttccttacctctgcgggtgaggccaaggcaagaaatggaatgttcatagttgggtgaaaacaaggaccttaggaacctggatttcccactatctgtgtcctgttcatgtctgttccccacacctgaaacagggcacacaggaggtgc

SEQ ID NO: 20 - DNA, 500 bp, Homo sapiens
aacgtgtctagagcacagataaactttactaaatctcagttggaaattgcatgacttctataaaaatgtcaaaatagaataggcaagggaagctcaatttgccgatgccagattcttgttaagaaagttttaatgatgctccctctgtttgtttgtttggttggttcctaggggaaaataagctggtaggggaccttctggtcttaggaggagccacactctatggtatttctaacgtctgggaagaatacatcatccgaactctgagccgagtggaattcctgggaatgattggtctctttggagcatttttcagtggaattcaattgtgagtaaaaataacaagaaatatagatagtagagggactgtcaagacttattcttacaatttttgttcttgggatgtaaaaatcaaggctggaatccagacaccagcttcctgtacaaaggacttatgtaatctgccaactcaatatctttattagtaaatcacctccaaa

SEQ ID NO: 21 - DNA, 500 bp, Homo sapiens
ccagggccccgtggcacaccctcacacaccgaccgggttcttcagggataaactgcactctgcctaattagacttgcttctgcatgacccaaaattctttctgaaattcatctggttgcttcattatttgtatacatctgataagtgggggggaaaaaagaaagaaagaaagtagagtgcattctaatcttgctcatcaggttcatttcctttgaatttgcagagctataatggagcataaggaactgttgaaggtgccctgggactggcaaataggtaaggagttggctcacatacgatgctctttttataacctgagaaatgtgtttgaagaatgatgtgggtcgggtggagcacaaacaaggaggggatttagagtggtaggggacaagggaaaatgaaatccctttgtcatttcaggaaatcctgcacactctgagagactaaattgtattattcatagtgtttcctaattgagcttctgcccattttcattgcca

SEQ ID NO: 22 - DNA, 500 bp, Homo sapiens
tttaatttttgtactgtgttatccaataataaacatcagtgaataggggaaaagagaaactgccttgtgtgagtgacaagtggtgtgcactcttccccttctttcctgctgtggatgctgaagatgccctggaggaggccctgacgctgccttctctttactcttcccccaggactgctctacgttggctttagtgcctgcatgtttggtctctacagctttatgccagtcgtcataaagaaaaccagtgccacttcagtcaacctctccttgctcacagcagacttgtacagcctgttctgtggattgtttctcttccactacaaggtaagttgagtgagtgctcctttgtgaagatgatactttgtaggaaacaatgaacatgggtaggaatgcctggatgtgaaatgccctgattttgtgtagggaatagtaatgtaactgggtgtgctaacctccatgatttaggtattcacatttcagctataagacagagacat

SEQ ID NO: 23 - DNA, 500 bp, Homo sapiens
tggccatgtttttgcccatcatctctgtattgtacatagagaaaggtatttactaggtgcagtttacacaggtggactgcaaggtagcaaatcctccaaaagcttgtgaaaggaacaagctcaacatcactggagacacaggctctaatccacctgacttggaaggatgcctagctaacgtgtatcctgatcacaactcccctgcattcattactgtgaaaatttttgaatcaaaagcaagtattattgttattatgatttgtattattattattgctgttactgttattacaccaactttcaggaggatgttttgtactcctgatggaaactattgccagacccacatgtagtcaacataaaccccacttttctatggcaattcactttttaagagtcatactttatttttttgcacagactatatgagtgatgcatgccacaggagctccacccctgagaagtttccttttcctagggacctggtggttaatggtttc

SEQ ID NO: 24 - DNA, 1000 bp, Homo sapiens
ggagttgggggactattaaaggagctgttaggctcccctgctgcgtggtttacctcatttaagtcttacaactcccctgggagaaagacatgagtagtctcgctgcacagagaagaatgtctcagaatttaattgcttagagtctctgcggataagtttcagatgtgccactgaaactcaaatctgcctacgttcctttcacagcactacattgcctcaaatggagacgttttgagttggtctgatatgctttttccagttcccagaattgattctttgtgagaagtagttttttctcccatttccaccaggccaattatggcgaatgtagtaggagcaacatctaggattctccgtttgggtttctaatccggggccggcacctgtctcggcgtcctgccgctgtcccaggccggtggtgctggattccgcggcgcttgggcagaaacgaggaatccccgccaactccacagggaggatctattcccagcccccttatccccctaaaaaacacccaaaccaaaacaatcaccacaacctagaccgtgggcctctgcggacggggcagtagagggtcgggggcaacaaggcacctcggcagcgccctctggcgagaggagagcgcgcaggtcccccgcccgcgccccgctctgccgccgccgccgccgccgccgccgccgccgccggcgcccccgcccggctctccgggttcaccctcggtctctcactccgtgagacacagacggtagctttccgaccgagcggggcacaggctgaggcggcgccagcacaactgccgccgcgcgccccgaggagacccgggcacgaggaaacaaggagaaatcaggactccttccccggaaccgaccggcagctccgcgcccccgcgcgacacccttcgcagccacttcgcggggcgggccaggacttggggacgcggctcgggaagagccggggcgggcggcggcggcggcggcacgggcgcgagggtgcgcgcact

SEQ ID NO: 25 - DNA, 17 bp, Homo sapiens
ccgcccggccgggtcct

SEQ ID NO: 26 - DNA, 17 bp, Homo sapiens
cgcccggccgggtcctc

SEQ ID NO: 27 - DNA, 17 bp, Homo sapiens
gcccggccgggtcctct

SEQ ID NO: 28 - DNA, 17 bp, Homo sapiens
cccggccgggtcctctg

SEQ ID NO: 29 - DNA, 17 bp, Homo sapiens
ccggccgggtcctctgc

SEQ ID NO: 30 - DNA, 17 bp, Homo sapiens
cggccgggtcctctgcg

SEQ ID NO: 31 - DNA, 17 bp, Homo sapiens
ggccgggtcctctgcgc

SEQ ID NO: 32 - DNA, 17 bp, Homo sapiens
gccgggtcctctgcgcg

SEQ ID NO: 33 - DNA, 17 bp, Homo sapiens
ccgggtcctctgcgcgt

SEQ ID NO: 34 - DNA, 17 bp, Homo sapiens
cgggtcctctgcgcgtt

SEQ ID NO: 35 - DNA, 17 bp, Homo sapiens
gggtcctctgcgcgttc

SEQ ID NO: 36 - DNA, 17 bp, Homo sapiens
ggtcctctgcgcgttcc

SEQ ID NO: 37 - DNA, 17 bp, Homo sapiens
gtcctctgcgcgttccc

SEQ ID NO: 38 - DNA, 17 bp, Homo sapiens
tcctctgcgcgttcccg

SEQ ID NO: 39 - DNA, 17 bp, Homo sapiens
cctctgcgcgttcccgg

SEQ ID NO: 40 - DNA, 17 bp, Homo sapiens
ctctgcgcgttcccggg

SEQ ID NO: 41 - DNA, 17 bp, Homo sapiens
tctgcgcgttcccgggc

SEQ ID NO: 42 - DNA, 17 bp, Homo sapiens
ctgcgcgttcccgggcc

SEQ ID NO: 43 - DNA, 17 bp, Homo sapiens
tgcgcgttcccgggccc

SEQ ID NO: 44 - DNA, 17 bp, Homo sapiens
gcgcgttcccgggcccg

SEQ ID NO: 45 - DNA, 17 bp, Homo sapiens
cgcgttcccgggcccgg

SEQ ID NO: 46 - DNA, 17 bp, Homo sapiens
gcgttcccgggcccgga

SEQ ID NO: 47 - DNA, 17 bp, Homo sapiens
cgttcccgggcccggaa

SEQ ID NO: 48 - DNA, 17 bp, Homo sapiens
gttcccgggcccggaac

SEQ ID NO: 49 - DNA, 17 bp, Homo sapiens
ttcccgggcccggaacc

SEQ ID NO: 50 - DNA, 17 bp, Homo sapiens
tcccgggcccggaaccg

SEQ ID NO: 51 - DNA, 17 bp, Homo sapiens
cccgggcccggaaccgg

SEQ ID NO: 52 - DNA, 17 bp, Homo sapiens
ccgggcccggaaccggc

SEQ ID NO: 53 - DNA, 17 bp, Homo sapiens
cgggcccggaaccggca

SEQ ID NO: 54 - DNA, 17 bp, Homo sapiens
gggcccggaaccggcac

SEQ ID NO: 55 - DNA, 17 bp, Homo sapiens
ggcccggaaccggcaca

SEQ ID NO: 56 - DNA, 17 bp, Homo sapiens
gcccggaaccggcacac

SEQ ID NO: 57 - DNA, 17 bp, Homo sapiens
cccggaaccggcacacg

SEQ ID NO: 58 - DNA, 17 bp, Homo sapiens
ccggaaccggcacacga

SEQ ID NO: 59 - DNA, 17 bp, Homo sapiens
cggaaccggcacacgat

SEQ ID NO: 60 - DNA, 17 bp, Homo sapiens
ggaaccggcacacgatg

SEQ ID NO: 61 - DNA, 17 bp, Homo sapiens
gaaccggcacacgatgc

SEQ ID NO: 62 - DNA, 25 bp, Homo sapiens
ccgcccggccgggtcctctgcgcgt

SEQ ID NO: 63 - DNA, 25 bp, Homo sapiens
cgcccggccgggtcctctgcgcgtt

SEQ ID NO: 64 - DNA, 25 bp, Homo sapiens
gcccggccgggtcctctgcgcgttc

SEQ ID NO: 65 - DNA, 25 bp, Homo sapiens
cccggccgggtcctctgcgcgttcc

SEQ ID NO: 66 - DNA, 25 bp, Homo sapiens
ccggccgggtcctctgcgcgttccc

SEQ ID NO: 67 - DNA, 25 bp, Homo sapiens
cggccgggtcctctgcgcgttcccg

SEQ ID NO: 68 - DNA, 25 bp, Homo sapiens
ggccgggtcctctgcgcgttcccgg

SEQ ID NO: 69 - DNA, 25 bp, Homo sapiens
gccgggtcctctgcgcgttcccggg

SEQ ID NO: 70 - DNA, 25 bp, Homo sapiens
ccgggtcctctgcgcgttcccgggc

SEQ ID NO: 71 - DNA, 25 bp, Homo sapiens
cgggtcctctgcgcgttcccgggcc

SEQ ID NO: 72 - DNA, 25 bp, Homo sapiens
gggtcctctgcgcgttcccgggccc

SEQ ID NO: 73 - DNA, 25 bp, Homo sapiens
ggtcctctgcgcgttcccgggcccg

SEQ ID NO: 74 - DNA, 25 bp, Homo sapiens
gtcctctgcgcgttcccgggcccgg

SEQ ID NO: 75 - DNA, 25 bp, Homo sapiens
tcctctgcgcgttcccgggcccgga

SEQ ID NO: 76 - DNA, 25 bp, Homo sapiens
cctctgcgcgttcccgggcccggaa

SEQ ID NO: 77 - DNA, 25 bp, Homo sapiens
ctctgcgcgttcccgggcccggaac

SEQ ID NO: 78 - DNA, 25 bp, Homo sapiens
tctgcgcgttcccgggcccggaacc

SEQ ID NO: 79 - DNA, 25 bp, Homo sapiens
ctgcgcgttcccgggcccggaaccg

SEQ ID NO: 80 - DNA, 25 bp, Homo sapiens
tgcgcgttcccgggcccggaaccgg

SEQ ID NO: 81 - DNA, 25 bp, Homo sapiens
gcgcgttcccgggcccggaaccggc

SEQ ID NO: 82 - DNA, 25 bp, Homo sapiens
cgcgttcccgggcccggaaccggca

SEQ ID NO: 83 - DNA, 25 bp, Homo sapiens
gcgttcccgggcccggaaccggcac

SEQ ID NO: 84 - DNA, 25 bp, Homo sapiens
cgttcccgggcccggaaccggcaca

SEQ ID NO: 85 - DNA, 25 bp, Homo sapiens
gttcccgggcccggaaccggcacac

SEQ ID NO: 86 - DNA, 25 bp, Homo sapiens
ttcccgggcccggaaccggcacacg

SEQ ID NO: 87 - DNA, 25 bp, Homo sapiens
tcccgggcccggaaccggcacacga

SEQ ID NO: 88 - DNA, 25 bp, Homo sapiens
cccgggcccggaaccggcacacgat

SEQ ID NO: 89 - DNA, 25 bp, Homo sapiens
ccgggcccggaaccggcacacgatg

SEQ ID NO: 90 - DNA, 25 bp, Homo sapiens
cgggcccggaaccggcacacgatgc

SEQ ID NO: 91 - DNA, 17 bp, Homo sapiens
ttcctgctgatccggta

SEQ ID NO: 92 - DNA, 17 bp, Homo sapiens
tcctgctgatccggtac

SEQ ID NO: 93 - DNA, 17 bp, Homo sapiens
cctgctgatccggtaca

SEQ ID NO: 94 - DNA, 17 bp, Homo sapiens
ctgctgatccggtacaa

SEQ ID NO: 95 - DNA, 17 bp, Homo sapiens
tgctgatccggtacaag

SEQ ID NO: 96 - DNA, 17 bp, Homo sapiens
gctgatccggtacaagg

SEQ ID NO: 97 - DNA, 17 bp, Homo sapiens
ctgatccggtacaaggc

SEQ ID NO: 98 - DNA, 17 bp, Homo sapiens
tgatccggtacaaggct

SEQ ID NO: 99 - DNA, 17 bp, Homo sapiens
gatccggtacaaggctg

SEQ ID NO: 100 - DNA, 17 bp, Homo sapiens
atccggtacaaggctgt

SEQ ID NO: 101 - DNA, 17 bp, Homo sapiens
tccggtacaaggctgtg

SEQ ID NO: 102 - DNA, 17 bp, Homo sapiens
ccggtacaaggctgtgc

SEQ ID NO: 103 - DNA, 17 bp, Homo sapiens
cggtacaaggctgtgca

SEQ ID NO: 104 - DNA, 17 bp, Homo sapiens
ggtacaaggctgtgcat

SEQ ID NO: 105 - DNA, 17 bp, Homo sapiens
gtacaaggctgtgcatt

SEQ ID NO: 106 - DNA, 17 bp, Homo sapiens
tacaaggctgtgcattt

SEQ ID NO: 107 - DNA, 17 bp, Homo sapiens
acaaggctgtgcatttc

SEQ ID NO: 108 - DNA, 17 bp, Homo sapiens
caaggctgtgcatttca

SEQ ID NO: 109 - DNA, 17 bp, Homo sapiens
aaggctgtgcatttcat

SEQ ID NO: 110 - DNA, 17 bp, Homo sapiens
aggctgtgcatttcatc

SEQ ID NO: 111 - DNA, 17 bp, Homo sapiens
ggctgtgcatttcatcg

SEQ ID NO: 112 - DNA, 17 bp, Homo sapiens
gctgtgcatttcatcgg

SEQ ID NO: 113 - DNA, 17 bp, Homo sapiens
ctgtgcatttcatcggc

SEQ ID NO: 114 - DNA, 17 bp, Homo sapiens
tgtgcatttcatcggca

SEQ ID NO: 115 - DNA, 17 bp, Homo sapiens
gtgcatttcatcggcat

SEQ ID NO: 116 - DNA, 17 bp, Homo sapiens
tgcatttcatcggcatc

SEQ ID NO: 117 - DNA, 17 bp, Homo sapiens
gcatttcatcggcatcg

SEQ ID NO: 118 - DNA, 17 bp, Homo sapiens
catttcatcggcatcgt

SEQ ID NO: 119 - DNA, 17 bp, Homo sapiens
atttcatcggcatcgtt

SEQ ID NO: 120 - DNA, 17 bp, Homo sapiens
tttcatcggcatcgttg

SEQ ID NO: 121 - DNA, 17 bp, Homo sapiens
ttcatcggcatcgttgt

SEQ ID NO: 122 - DNA, 17 bp, Homo sapiens
tcatcggcatcgttgtc

SEQ ID NO: 123 - DNA, 17 bp, Homo sapiens
catcggcatcgttgtct

SEQ ID NO: 124 - DNA, 17 bp, Homo sapiens
atcggcatcgttgtctg

SEQ ID NO: 125 - DNA, 17 bp, Homo sapiens
tcggcatcgttgtctgc

SEQ ID NO: 126 - DNA, 17 bp, Homo sapiens
cggcatcgttgtctgca

SEQ ID NO: 127 - DNA, 17 bp, Homo sapiens
ggcatcgttgtctgcat

SEQ ID NO: 128 - DNA, 17 bp, Homo sapiens
gcatcgttgtctgcatc

SEQ ID NO: 129 - DNA, 17 bp, Homo sapiens
catcgttgtctgcatcc

SEQ ID NO: 130 - DNA, 17 bp, Homo sapiens
atcgttgtctgcatcct

SEQ ID NO: 131 - DNA, 17 bp, Homo sapiens
tcgttgtctgcatcctg

SEQ ID NO: 132 - DNA, 17 bp, Homo sapiens
cgttgtctgcatcctgg

SEQ ID NO: 133 - DNA, 17 bp, Homo sapiens
gttgtctgcatcctggg

SEQ ID NO: 134 - DNA, 17 bp, Homo sapiens
ttgtctgcatcctggga

SEQ ID NO: 135 - DNA, 17 bp, Homo sapiens
tgtctgcatcctgggaa

SEQ ID NO: 136 - DNA, 17 bp, Homo sapiens
gtctgcatcctgggaat

SEQ ID NO: 137 - DNA, 17 bp, Homo sapiens
tctgcatcctgggaatg

SEQ ID NO: 138 - DNA, 17 bp, Homo sapiens
ctgcatcctgggaatgg

SEQ ID NO: 139 - DNA, 17 bp, Homo sapiens
tgcatcctgggaatggg

SEQ ID NO: 140 - DNA, 17 bp, Homo sapiens
gcatcctgggaatgggc

SEQ ID NO: 141 - DNA, 17 bp, Homo sapiens
catcctgggaatgggct

SEQ ID NO: 142 - DNA, 17 bp, Homo sapiens
atcctgggaatgggctg

SEQ ID NO: 143 - DNA, 17 bp, Homo sapiens
tcctgggaatgggctgc

SEQ ID NO: 144 - DNA, 17 bp, Homo sapiens
cctgggaatgggctgca

SEQ ID NO: 145 - DNA, 17 bp, Homo sapiens
ctgggaatgggctgcat

SEQ ID NO: 146 - DNA, 17 bp, Homo sapiens
tgggaatgggctgcatg

SEQ ID NO: 147 - DNA, 17 bp, Homo sapiens
gggaatgggctgcatgg

SEQ ID NO: 148 - DNA, 17 bp, Homo sapiens
ggaatgggctgcatggt

SEQ ID NO: 149 - DNA, 17 bp, Homo sapiens
gaatgggctgcatggtg

SEQ ID NO: 150 - DNA, 17 bp, Homo sapiens
aatgggctgcatggtgg

SEQ ID NO: 151 - DNA, 17 bp, Homo sapiens
atgggctgcatggtggg

SEQ ID NO: 152 - DNA, 17 bp, Homo sapiens
tgggctgcatggtggga

SEQ ID NO: 153 - DNA, 17 bp, Homo sapiens
gggctgcatggtgggag

SEQ ID NO: 154 - DNA, 17 bp, Homo sapiens
ggctgcatggtgggagc

SEQ ID NO: 155 - DNA, 17 bp, Homo sapiens
gctgcatggtgggagca

SEQ ID NO: 156 - DNA, 17 bp, Homo sapiens
ctgcatggtgggagcag

SEQ ID NO: 157 - DNA, 17 bp, Homo sapiens
tgcatggtgggagcaga

SEQ ID NO: 158 - DNA, 17 bp, Homo sapiens
gcatggtgggagcagat

SEQ ID NO: 159 - DNA, 17 bp, Homo sapiens
catggtgggagcagatg

SEQ ID NO: 160 - DNA, 17 bp, Homo sapiens
atggtgggagcagatgt

SEQ ID NO: 161 - DNA, 17 bp, Homo sapiens
tggtgggagcagatgtg

SEQ ID NO: 162 - DNA, 17 bp, Homo sapiens
ggtgggagcagatgtgc

SEQ ID NO: 163 - DNA, 17 bp, Homo sapiens
gtgggagcagatgtgct

SEQ ID NO: 164 - DNA, 17 bp, Homo sapiens
tgggagcagatgtgctt

SEQ ID NO: 165 - DNA, 17 bp, Homo sapiens
gggagcagatgtgcttg

SEQ ID NO: 166 - DNA, 17 bp, Homo sapiens
ggagcagatgtgcttgt

SEQ ID NO: 167 - DNA, 17 bp, Homo sapiens
gagcagatgtgcttgtg

SEQ ID NO: 168 - DNA, 17 bp, Homo sapiens
agcagatgtgcttgtgg

SEQ ID NO: 169 - DNA, 17 bp, Homo sapiens
gcagatgtgcttgtggg

SEQ ID NO: 170 - DNA, 17 bp, Homo sapiens
cagatgtgcttgtggga

SEQ ID NO: 171 - DNA, 17 bp, Homo sapiens
agatgtgcttgtgggaa

SEQ ID NO: 172 - DNA, 17 bp, Homo sapiens
gatgtgcttgtgggaag

SEQ ID NO: 173 - DNA, 17 bp, Homo sapiens
atgtgcttgtgggaaga

SEQ ID NO: 174 - DNA, 17 bp, Homo sapiens
tgtgcttgtgggaagac

SEQ ID NO: 175 - DNA, 17 bp, Homo sapiens
gtgcttgtgggaagaca

SEQ ID NO: 176 - DNA, 17 bp, Homo sapiens
tgcttgtgggaagacat

SEQ ID NO: 177 - DNA, 17 bp, Homo sapiens
gcttgtgggaagacatc

SEQ ID NO: 178 - DNA, 17 bp, Homo sapiens
cttgtgggaagacatca

SEQ ID NO: 179 - DNA, 17 bp, Homo sapiens
ttgtgggaagacatcag

SEQ ID NO: 180 - DNA, 17 bp, Homo sapiens
tgtgggaagacatcagg

SEQ ID NO: 181 - DNA, 17 bp, Homo sapiens
gtgggaagacatcaggg

SEQ ID NO: 182 - DNA, 17 bp, Homo sapiens
tgggaagacatcaggga

SEQ ID NO: 183 - DNA, 17 bp, Homo sapiens
gggaagacatcagggag

SEQ ID NO: 184 - DNA, 17 bp, Homo sapiens
ggaagacatcagggagc

SEQ ID NO: 185 - DNA, 17 bp, Homo sapiens
gaagacatcagggagca

SEQ ID NO: 186 - DNA, 17 bp, Homo sapiens
aagacatcagggagcag

SEQ ID NO: 187 - DNA, 17 bp, Homo sapiens
agacatcagggagcagg

SEQ ID NO: 188 - DNA, 17 bp, Homo sapiens
gacatcagggagcaggg

SEQ ID NO: 189 - DNA, 17 bp, Homo sapiens
acatcagggagcagggg

SEQ ID NO: 190 - DNA, 17 bp, Homo sapiens
catcagggagcagggga

SEQ ID NO: 191 - DNA, 17 bp, Homo sapiens
atcagggagcaggggaa

SEQ ID NO: 192 - DNA, 17 bp, Homo sapiens
tcagggagcaggggaaa

SEQ ID NO: 193 - DNA, 17 bp, Homo sapiens
cagggagcaggggaaaa

SEQ ID NO: 194 - DNA, 17 bp, Homo sapiens
agggagcaggggaaaat

SEQ ID NO: 195 - DNA, 17 bp, Homo sapiens
gggagcaggggaaaata

SEQ ID NO: 196 - DNA, 17 bp, Homo sapiens
ggagcaggggaaaataa

SEQ ID NO: 197 - DNA, 17 bp, Homo sapiens
gagcaggggaaaataag

SEQ ID NO: 198 - DNA, 17 bp, Homo sapiens
agcaggggaaaataagc

SEQ ID NO: 199 - DNA, 17 bp, Homo sapiens
gcaggggaaaataagct

SEQ ID NO: 200 - DNA, 17 bp, Homo sapiens
caggggaaaataagctg

SEQ ID NO: 201 - DNA, 17 bp, Homo sapiens
aggggaaaataagctgg

SEQ ID NO: 202 - DNA, 17 bp, Homo sapiens
ggggaaaataagctggt

SEQ ID NO: 203 - DNA, 17 bp, Homo sapiens
gggaaaataagctggta

SEQ ID NO: 204 - DNA, 17 bp, Homo sapiens
ggaaaataagctggtag

SEQ ID NO: 205 - DNA, 17 bp, Homo sapiens
gaaaataagctggtagg

SEQ ID NO: 206 - DNA, 17 bp, Homo sapiens
aaaataagctggtaggg

SEQ ID NO: 207 - DNA, 17 bp, Homo sapiens
aaataagctggtagggg

SEQ ID NO: 208 - DNA, 17 bp, Homo sapiens
aataagctggtagggga

SEQ ID NO: 209 - DNA, 17 bp, Homo sapiens
ataagctggtaggggac

SEQ ID NO: 210 - DNA, 17 bp, Homo sapiens
taagctggtaggggacc

SEQ ID NO: 211 - DNA, 17 bp, Homo sapiens
aagctggtaggggacct

SEQ ID NO: 212 - DNA, 17 bp, Homo sapiens
agctggtaggggacctt

SEQ ID NO: 213 - DNA, 17 bp, Homo sapiens
gctggtaggggaccttc

SEQ ID NO: 214 - DNA, 17 bp, Homo sapiens
ctggtaggggaccttct

SEQ ID NO: 215 - DNA, 17 bp, Homo sapiens
tggtaggggaccttctg

SEQ ID NO: 216 - DNA, 17 bp, Homo sapiens
ggtaggggaccttctgg

SEQ ID NO: 217 - DNA, 17 bp, Homo sapiens
gtaggggaccttctggt

SEQ ID NO: 218 - DNA, 17 bp, Homo sapiens
taggggaccttctggtc

SEQ ID NO: 219 - DNA, 17 bp, Homo sapiens
aggggaccttctggtct

SEQ ID NO: 220 - DNA, 17 bp, Homo sapiens
ggggaccttctggtctt

SEQ ID NO: 221 - DNA, 17 bp, Homo sapiens
gggaccttctggtctta

SEQ ID NO: 222 - DNA, 17 bp, Homo sapiens
ggaccttctggtcttag

SEQ ID NO: 223 - DNA, 17 bp, Homo sapiens
gaccttctggtcttagg

SEQ ID NO: 224 - DNA, 17 bp, Homo sapiens
accttctggtcttagga

SEQ ID NO: 225 - DNA, 17 bp, Homo sapiens
ccttctggtcttaggag

SEQ ID NO: 226 - DNA, 17 bp, Homo sapiens
cttctggtcttaggagg

SEQ ID NO: 227 - DNA, 17 bp, Homo sapiens
ttctggtcttaggagga

SEQ ID NO: 228 - DNA, 17 bp, Homo sapiens
tctggtcttaggaggag

SEQ ID NO: 229 - DNA, 17 bp, Homo sapiens
ctggtcttaggaggagc

SEQ ID NO: 230 - DNA, 17 bp, Homo sapiens
tggtcttaggaggagcc

SEQ ID NO: 231 - DNA, 17 bp, Homo sapiens
ggtcttaggaggagcca

SEQ ID NO: 232 - DNA, 17 bp, Homo sapiens
gtcttaggaggagccac

SEQ ID NO: 233 - DNA, 17 bp, Homo sapiens
tcttaggaggagccaca

SEQ ID NO: 234 - DNA, 17 bp, Homo sapiens
cttaggaggagccacac

SEQ ID NO: 235 - DNA, 17 bp, Homo sapiens
ttaggaggagccacact

SEQ ID NO: 236 - DNA, 17 bp, Homo sapiens
taggaggagccacactc

SEQ ID NO: 237 - DNA, 17 bp, Homo sapiens
aggaggagccacactct

SEQ ID NO: 238 - DNA, 17 bp, Homo sapiens
ggaggagccacactcta

SEQ ID NO: 239 - DNA, 17 bp, Homo sapiens
gaggagccacactctat

SEQ ID NO: 240 - DNA, 17 bp, Homo sapiens
aggagccacactctatg

SEQ ID NO: 241 - DNA, 17 bp, Homo sapiens
ggagccacactctatgg

SEQ ID NO: 242 - DNA, 17 bp, Homo sapiens
gagccacactctatggt

SEQ ID NO: 243 - DNA, 17 bp, Homo sapiens
agccacactctatggta

SEQ ID NO: 244 - DNA, 17 bp, Homo sapiens
gccacactctatggtat

SEQ ID NO: 245 - DNA, 17 bp, Homo sapiens
ccacactctatggtatt

SEQ ID NO: 246 - DNA, 17 bp, Homo sapiens
cacactctatggtattt

SEQ ID NO: 247 - DNA, 17 bp, Homo sapiens
acactctatggtatttc

SEQ ID NO: 248 - DNA, 17 bp, Homo sapiens
cactctatggtatttct

SEQ ID NO: 249 - DNA, 17 bp, Homo sapiens
actctatggtatttcta

SEQ ID NO: 250 - DNA, 17 bp, Homo sapiens
ctctatggtatttctaa

SEQ ID NO: 251 - DNA, 17 bp, Homo sapiens
tctatggtatttctaac

SEQ ID NO: 252 - DNA, 17 bp, Homo sapiens
ctatggtatttctaacg

SEQ ID NO: 253 - DNA, 17 bp, Homo sapiens
tatggtatttctaacgt

SEQ ID NO: 254 - DNA, 17 bp, Homo sapiens
atggtatttctaacgtc

SEQ ID NO: 255 - DNA, 17 bp, Homo sapiens
tggtatttctaacgtct

SEQ ID NO: 256 - DNA, 17 bp, Homo sapiens
ggtatttctaacgtctg

SEQ ID NO: 257 - DNA, 17 bp, Homo sapiens
gtatttctaacgtctgg

SEQ ID NO: 258 - DNA, 17 bp, Homo sapiens
tatttctaacgtctggg

SEQ ID NO: 259 - DNA, 17 bp, Homo sapiens
atttctaacgtctggga

SEQ ID NO: 260 - DNA, 17 bp, Homo sapiens
tttctaacgtctgggaa

SEQ ID NO: 261 - DNA, 17 bp, Homo sapiens
ttctaacgtctgggaag

SEQ ID NO: 262 - DNA, 17 bp, Homo sapiens
tctaacgtctgggaaga

SEQ ID NO: 263 - DNA, 17 bp, Homo sapiens
ctaacgtctgggaagaa

SEQ ID NO: 264 - DNA, 17 bp, Homo sapiens
taacgtctgggaagaat

SEQ ID NO: 265 - DNA, 17 bp, Homo sapiens
aacgtctgggaagaata

SEQ ID NO: 266 - DNA, 17 bp, Homo sapiens
acgtctgggaagaatac

SEQ ID NO: 267 - DNA, 17 bp, Homo sapiens
cgtctgggaagaataca

SEQ ID NO: 268 - DNA, 17 bp, Homo sapiens
gtctgggaagaatacat

SEQ ID NO: 269 - DNA, 17 bp, Homo sapiens
tctgggaagaatacatc

SEQ ID NO: 270 - DNA, 17 bp, Homo sapiens
ctgggaagaatacatca

SEQ ID NO: 271 - DNA, 17 bp, Homo sapiens
tgggaagaatacatcat

SEQ ID NO: 272 - DNA, 17 bp, Homo sapiens
gggaagaatacatcatc

SEQ ID NO: 273 - DNA, 17 bp, Homo sapiens
ggaagaatacatcatcc

SEQ ID NO: 274 - DNA, 17 bp, Homo sapiens
gaagaatacatcatccg

SEQ ID NO: 275 - DNA, 17 bp, Homo sapiens
aagaatacatcatccga

SEQ ID NO: 276 - DNA, 17 bp, Homo sapiens
agaatacatcatccgaa

SEQ ID NO: 277 - DNA, 17 bp, Homo sapiens
gaatacatcatccgaac

SEQ ID NO: 278 - DNA, 17 bp, Homo sapiens
aatacatcatccgaact

SEQ ID NO: 279 - DNA, 17 bp, Homo sapiens
atacatcatccgaactc

SEQ ID NO: 280 - DNA, 17 bp, Homo sapiens
tacatcatccgaactct

SEQ ID NO: 281 - DNA, 17 bp, Homo sapiens
acatcatccgaactctg

SEQ ID NO: 282 - DNA, 17 bp, Homo sapiens
catcatccgaactctga

SEQ ID NO: 283 - DNA, 17 bp, Homo sapiens
atcatccgaactctgag

SEQ ID NO: 284 - DNA, 17 bp, Homo sapiens
tcatccgaactctgagc

SEQ ID NO: 285 - DNA, 17 bp, Homo sapiens
catccgaactctgagcc

SEQ ID NO: 286 - DNA, 17 bp, Homo sapiens
atccgaactctgagccg

SEQ ID NO: 287 - DNA, 17 bp, Homo sapiens
tccgaactctgagccga

SEQ ID NO: 288 - DNA, 17 bp, Homo sapiens
ccgaactctgagccgag

SEQ ID NO: 289 - DNA, 17 bp, Homo sapiens
cgaactctgagccgagt

SEQ ID NO: 290 - DNA, 17 bp, Homo sapiens
gaactctgagccgagtg

SEQ ID NO: 291 - DNA, 17 bp, Homo sapiens
aactctgagccgagtgg

SEQ ID NO: 292 - DNA, 17 bp, Homo sapiens
actctgagccgagtgga

SEQ ID NO: 293 - DNA, 17 bp, Homo sapiens
ctctgagccgagtggaa

SEQ ID NO: 294 - DNA, 17 bp, Homo sapiens
tctgagccgagtggaat

SEQ ID NO: 295 - DNA, 17 bp, Homo sapiens
ctgagccgagtggaatt

SEQ ID NO: 296 - DNA, 17 bp, Homo sapiens
tgagccgagtggaattc

SEQ ID NO: 297 - DNA, 17 bp, Homo sapiens
gagccgagtggaattcc

SEQ ID NO: 298 - DNA, 17 bp, Homo sapiens
agccgagtggaattcct

SEQ ID NO: 299 - DNA, 17 bp, Homo sapiens
gccgagtggaattcctg

SEQ ID NO: 300 - DNA, 17 bp, Homo sapiens
ccgagtggaattcctgg

SEQ ID NO: 301 - DNA, 17 bp, Homo sapiens
cgagtggaattcctggg

SEQ ID NO: 302 - DNA, 17 bp, Homo sapiens
gagtggaattcctggga

SEQ ID NO: 303 - DNA, 17 bp, Homo sapiens
agtggaattcctgggaa

SEQ ID NO: 304 - DNA, 17 bp, Homo sapiens
gtggaattcctgggaat

SEQ ID NO: 305 - DNA, 17 bp, Homo sapiens
tggaattcctgggaatg

SEQ ID NO: 306 - DNA, 17 bp, Homo sapiens
ggaattcctgggaatga

SEQ ID NO: 307 - DNA, 17 bp, Homo sapiens
gaattcctgggaatgat

SEQ ID NO: 308 - DNA, 17 bp, Homo sapiens
aattcctgggaatgatt

SEQ ID NO: 309 - DNA, 17 bp, Homo sapiens
attcctgggaatgattg

SEQ ID NO: 310 - DNA, 17 bp, Homo sapiens
ttcctgggaatgattgg

SEQ ID NO: 311 - DNA, 17 bp, Homo sapiens
tcctgggaatgattggt

SEQ ID NO: 312 - DNA, 17 bp, Homo sapiens
cctgggaatgattggtc

SEQ ID NO: 313 - DNA, 17 bp, Homo sapiens
ctgggaatgattggtct

SEQ ID NO: 314 - DNA, 17 bp, Homo sapiens
tgggaatgattggtctc

SEQ ID NO: 315 - DNA, 17 bp, Homo sapiens
gggaatgattggtctct

SEQ ID NO: 316 - DNA, 17 bp, Homo sapiens
ggaatgattggtctctt

SEQ ID NO: 317 - DNA, 17 bp, Homo sapiens
gaatgattggtctcttt

SEQ ID NO: 318 - DNA, 17 bp, Homo sapiens
aatgattggtctctttg

SEQ ID NO: 319 - DNA, 17 bp, Homo sapiens
atgattggtctctttgg

SEQ ID NO: 320 - DNA, 17 bp, Homo sapiens
tgattggtctctttgga

SEQ ID NO: 321 - DNA, 17 bp, Homo sapiens
gattggtctctttggag

SEQ ID NO: 322 - DNA, 17 bp, Homo sapiens
attggtctctttggagc

SEQ ID NO: 323 - DNA, 17 bp, Homo sapiens
ttggtctctttggagca

SEQ ID NO: 324 - DNA, 17 bp, Homo sapiens
tggtctctttggagcat

SEQ ID NO: 325 - DNA, 17 bp, Homo sapiens
ggtctctttggagcatt

SEQ ID NO: 326 - DNA, 17 bp, Homo sapiens
gtctctttggagcattt

SEQ ID NO: 327 - DNA, 17 bp, Homo sapiens
tctctttggagcatttt

SEQ ID NO: 328 - DNA, 17 bp, Homo sapiens
ctctttggagcattttt

SEQ ID NO: 329 - DNA, 17 bp, Homo sapiens
tctttggagcatttttc

SEQ ID NO: 330 - DNA, 17 bp, Homo sapiens
ctttggagcatttttca

SEQ ID NO: 331 - DNA, 17 bp, Homo sapiens
tttggagcatttttcag

SEQ ID NO: 332 - DNA, 17 bp, Homo sapiens
ttggagcatttttcagt

SEQ ID NO: 333 - DNA, 17 bp, Homo sapiens
tggagcatttttcagtg

SEQ ID NO: 334 - DNA, 17 bp, Homo sapiens
ggagcatttttcagtgg

SEQ ID NO: 335 - DNA, 17 bp, Homo sapiens
gagcatttttcagtgga

SEQ ID NO: 336 - DNA, 17 bp, Homo sapiens
agcatttttcagtggaa

SEQ ID NO: 337 - DNA, 17 bp, Homo sapiens
gcatttttcagtggaat

SEQ ID NO: 338 - DNA, 17 bp, Homo sapiens
catttttcagtggaatt

SEQ ID NO: 339 - DNA, 17 bp, Homo sapiens
atttttcagtggaattc

SEQ ID NO: 340 - DNA, 17 bp, Homo sapiens
tttttcagtggaattca

SEQ ID NO: 341 - DNA, 17 bp, Homo sapiens
ttttcagtggaattcaa

SEQ ID NO: 342 - DNA, 17 bp, Homo sapiens
tttcagtggaattcaat

SEQ ID NO: 343 - DNA, 17 bp, Homo sapiens
ttcagtggaattcaatt

SEQ ID NO: 344 - DNA, 17 bp, Homo sapiens
tcagtggaattcaatta

SEQ ID NO: 345 - DNA, 17 bp, Homo sapiens
cagtggaattcaattag

SEQ ID NO: 346 - DNA, 17 bp, Homo sapiens
agtggaattcaattagc

SEQ ID NO: 347 - DNA, 17 bp, Homo sapiens
gtggaattcaattagct

SEQ ID NO: 348 - DNA, 17 bp, Homo sapiens
tggaattcaattagcta

SEQ ID NO: 349 - DNA, 17 bp, Homo sapiens
ggaattcaattagctat

SEQ ID NO: 350 - DNA, 17 bp, Homo sapiens
gaattcaattagctata

SEQ ID NO: 351 - DNA, 17 bp, Homo sapiens
aattcaattagctataa

SEQ ID NO: 352 - DNA, 17 bp, Homo sapiens
attcaattagctataat

SEQ ID NO: 353 - DNA, 17 bp, Homo sapiens
ttcaattagctataatg

SEQ ID NO: 354 - DNA, 17 bp, Homo sapiens
tcaattagctataatgg

SEQ ID NO: 355 - DNA, 17 bp, Homo sapiens
caattagctataatgga

SEQ ID NO: 356 - DNA, 17 bp, Homo sapiens
aattagctataatggag

SEQ ID NO: 357 - DNA, 17 bp, Homo sapiens
attagctataatggagc

SEQ ID NO: 358 - DNA, 17 bp, Homo sapiens
ttagctataatggagca

SEQ ID NO: 359 - DNA, 17 bp, Homo sapiens
tagctataatggagcat

SEQ ID NO: 360 - DNA, 17 bp, Homo sapiens
agctataatggagcata

SEQ ID NO: 361 - DNA, 17 bp, Homo sapiens
gctataatggagcataa

SEQ ID NO: 362 - DNA, 17 bp, Homo sapiens
ctataatggagcataag

SEQ ID NO: 363 - DNA, 17 bp, Homo sapiens
tataatggagcataagg

SEQ ID NO: 364 - DNA, 17 bp, Homo sapiens
ataatggagcataagga

SEQ ID NO: 365 - DNA, 17 bp, Homo sapiens
taatggagcataaggaa

SEQ ID NO: 366 - DNA, 17 bp, Homo sapiens
aatggagcataaggaac

SEQ ID NO: 367 - DNA, 17 bp, Homo sapiens
atggagcataaggaact

SEQ ID NO: 368 - DNA, 17 bp, Homo sapiens
tggagcataaggaactg

SEQ ID NO: 369 - DNA, 17 bp, Homo sapiens
ggagcataaggaactgt

SEQ ID NO: 370 - DNA, 17 bp, Homo sapiens
gagcataaggaactgtt

SEQ ID NO: 371 - DNA, 17 bp, Homo sapiens
agcataaggaactgttg

SEQ ID NO: 372 - DNA, 17 bp, Homo sapiens
gcataaggaactgttga

SEQ ID NO: 373 - DNA, 17 bp, Homo sapiens
cataaggaactgttgaa

SEQ ID NO: 374 - DNA, 17 bp, Homo sapiens
ataaggaactgttgaag

SEQ ID NO: 375 - DNA, 17 bp, Homo sapiens
taaggaactgttgaagg

SEQ ID NO: 376 - DNA, 17 bp, Homo sapiens
aaggaactgttgaaggt

SEQ ID NO: 377 - DNA, 17 bp, Homo sapiens
aggaactgttgaaggtg

SEQ ID NO: 378 - DNA, 17 bp, Homo sapiens
ggaactgttgaaggtgc

SEQ ID NO: 379 - DNA, 17 bp, Homo sapiens
gaactgttgaaggtgcc

SEQ ID NO: 380 - DNA, 17 bp, Homo sapiens
aactgttgaaggtgccc

SEQ ID NO: 381 - DNA, 17 bp, Homo sapiens
actgttgaaggtgccct

SEQ ID NO: 382 - DNA, 17 bp, Homo sapiens
ctgttgaaggtgccctg

SEQ ID NO: 383 - DNA, 17 bp, Homo sapiens
tgttgaaggtgccctgg

SEQ ID NO: 384 - DNA, 17 bp, Homo sapiens
gttgaaggtgccctggg

SEQ ID NO: 385 - DNA, 17 bp, Homo sapiens
ttgaaggtgccctggga

SEQ ID NO: 386 - DNA, 17 bp, Homo sapiens
tgaaggtgccctgggac

SEQ ID NO: 387 - DNA, 17 bp, Homo sapiens
gaaggtgccctgggact

SEQ ID NO: 388 - DNA, 17 bp, Homo sapiens
aaggtgccctgggactg

SEQ ID NO: 389 - DNA, 17 bp, Homo sapiens
aggtgccctgggactgg

SEQ ID NO: 390 - DNA, 17 bp, Homo sapiens
ggtgccctgggactggc

SEQ ID NO: 391 - DNA, 17 bp, Homo sapiens
gtgccctgggactggca

SEQ ID NO: 392 - DNA, 17 bp, Homo sapiens
tgccctgggactggcaa

SEQ ID NO: 393 - DNA, 17 bp, Homo sapiens
gccctgggactggcaaa

SEQ ID NO: 394 - DNA, 17 bp, Homo sapiens
ccctgggactggcaaat

SEQ ID NO: 395 - DNA, 17 bp, Homo sapiens
cctgggactggcaaata

SEQ ID NO: 396 - DNA, 17 bp, Homo sapiens
ctgggactggcaaatag

SEQ ID NO: 397 - DNA, 17 bp, Homo sapiens
tgggactggcaaatagg

SEQ ID NO: 398 - DNA, 17 bp, Homo sapiens
gggactggcaaatagga

SEQ ID NO: 399 - DNA, 17 bp, Homo sapiens
ggactggcaaataggac

SEQ ID NO: 400 - DNA, 17 bp, Homo sapiens
gactggcaaataggact

SEQ ID NO: 401 - DNA, 17 bp, Homo sapiens
actggcaaataggactg

SEQ ID NO: 402 - DNA, 17 bp, Homo sapiens
ctggcaaataggactgc

SEQ ID NO: 403 - DNA, 17 bp, Homo sapiens
tggcaaataggactgct

SEQ ID NO: 404 - DNA, 17 bp, Homo sapiens
ggcaaataggactgctc

SEQ ID NO: 405 - DNA, 17 bp, Homo sapiens
gcaaataggactgctct

SEQ ID NO: 406 - DNA, 17 bp, Homo sapiens
caaataggactgctcta

SEQ ID NO: 407 - DNA, 17 bp, Homo sapiens
aaataggactgctctac

SEQ ID NO: 408 - DNA, 17 bp, Homo sapiens
aataggactgctctacg

SEQ ID NO: 409 - DNA, 17 bp, Homo sapiens
ataggactgctctacgt

SEQ ID NO: 410 - DNA, 17 bp, Homo sapiens
taggactgctctacgtt

SEQ ID NO: 411 - DNA, 17 bp, Homo sapiens
aggactgctctacgttg

SEQ ID NO: 412 - DNA, 17 bp, Homo sapiens
ggactgctctacgttgg

SEQ ID NO: 413 - DNA, 17 bp, Homo sapiens
gactgctctacgttggc

SEQ ID NO: 414 - DNA, 17 bp, Homo sapiens
actgctctacgttggct

SEQ ID NO: 415 - DNA, 17 bp, Homo sapiens
ctgctctacgttggctt

SEQ ID NO: 416 - DNA, 17 bp, Homo sapiens
tgctctacgttggcttt

SEQ ID NO: 417 - DNA, 17 bp, Homo sapiens
gctctacgttggcttta

SEQ ID NO: 418 - DNA, 17 bp, Homo sapiens
ctctacgttggctttag

SEQ ID NO: 419 - DNA, 17 bp, Homo sapiens
tctacgttggctttagt

SEQ ID NO: 420 - DNA, 17 bp, Homo sapiens
ctacgttggctttagtg

SEQ ID NO: 421 - DNA, 17 bp, Homo sapiens
tacgttggctttagtgc

SEQ ID NO: 422 - DNA, 17 bp, Homo sapiens
acgttggctttagtgcc

SEQ ID NO: 423 - DNA, 17 bp, Homo sapiens
cgttggctttagtgcct

SEQ ID NO: 424 - DNA, 17 bp, Homo sapiens
gttggctttagtgcctg

SEQ ID NO: 425 - DNA, 17 bp, Homo sapiens
ttggctttagtgcctgc

SEQ ID NO: 426 - DNA, 17 bp, Homo sapiens
tggctttagtgcctgca

SEQ ID NO: 427 - DNA, 17 bp, Homo sapiens
ggctttagtgcctgcat

SEQ ID NO: 428 - DNA, 17 bp, Homo sapiens
gctttagtgcctgcatg

SEQ ID NO: 429 - DNA, 17 bp, Homo sapiens
ctttagtgcctgcatgt

SEQ ID NO: 430 - DNA, 17 bp, Homo sapiens
tttagtgcctgcatgtt

SEQ ID NO: 431 - DNA, 17 bp, Homo sapiens
ttagtgcctgcatgttt

SEQ ID NO: 432 - DNA, 17 bp, Homo sapiens
tagtgcctgcatgtttg

SEQ ID NO: 433 - DNA, 17 bp, Homo sapiens
agtgcctgcatgtttgg

SEQ ID NO: 434 - DNA, 17 bp, Homo sapiens
gtgcctgcatgtttggt

SEQ ID NO: 435 - DNA, 17 bp, Homo sapiens
tgcctgcatgtttggtc

SEQ ID NO: 436 - DNA, 17 bp, Homo sapiens
gcctgcatgtttggtct

SEQ ID NO: 437 - DNA, 17 bp, Homo sapiens
cctgcatgtttggtctc

SEQ ID NO: 438 - DNA, 17 bp, Homo sapiens
ctgcatgtttggtctct

SEQ ID NO: 439 - DNA, 17 bp, Homo sapiens
tgcatgtttggtctcta

SEQ ID NO: 440 - DNA, 17 bp, Homo sapiens
gcatgtttggtctctac

SEQ ID NO: 441 - DNA, 17 bp, Homo sapiens
catgtttggtctctaca

SEQ ID NO: 442 - DNA, 17 bp, Homo sapiens
atgtttggtctctacag

SEQ ID NO: 443 - DNA, 17 bp, Homo sapiens
tgtttggtctctacagc

SEQ ID NO: 444 - DNA, 17 bp, Homo sapiens
gtttggtctctacagct

SEQ ID NO: 445 - DNA, 17 bp, Homo sapiens
tttggtctctacagctt

SEQ ID NO: 446 - DNA, 17 bp, Homo sapiens
ttggtctctacagcttt

SEQ ID NO: 447 - DNA, 17 bp, Homo sapiens
tggtctctacagcttta

SEQ ID NO: 448 - DNA, 17 bp, Homo sapiens
ggtctctacagctttat

SEQ ID NO: 449 - DNA, 17 bp, Homo sapiens
gtctctacagctttatg

SEQ ID NO: 450 - DNA, 17 bp, Homo sapiens
tctctacagctttatgc

SEQ ID NO: 451 - DNA, 17 bp, Homo sapiens
ctctacagctttatgcc

SEQ ID NO: 452 - DNA, 17 bp, Homo sapiens
tctacagctttatgcca

SEQ ID NO: 453 - DNA, 17 bp, Homo sapiens
ctacagctttatgccag

SEQ ID NO: 454 - DNA, 17 bp, Homo sapiens
tacagctttatgccagt

SEQ ID NO: 455 - DNA, 17 bp, Homo sapiens
acagctttatgccagtc

SEQ ID NO: 456 - DNA, 17 bp, Homo sapiens
cagctttatgccagtcg

SEQ ID NO: 457 - DNA, 17 bp, Homo sapiens
agctttatgccagtcgt

SEQ ID NO: 458 - DNA, 17 bp, Homo sapiens
gctttatgccagtcgtc

SEQ ID NO: 459 - DNA, 17 bp, Homo sapiens
ctttatgccagtcgtca

SEQ ID NO: 460 - DNA, 17 bp, Homo sapiens
tttatgccagtcgtcat

SEQ ID NO: 461 - DNA, 17 bp, Homo sapiens
ttatgccagtcgtcata

SEQ ID NO: 462 - DNA, 17 bp, Homo sapiens
tatgccagtcgtcataa

SEQ ID NO: 463 - DNA, 17 bp, Homo sapiens
atgccagtcgtcataaa

SEQ ID NO: 464 - DNA, 17 bp, Homo sapiens
tgccagtcgtcataaag

SEQ ID NO: 465 - DNA, 17 bp, Homo sapiens
gccagtcgtcataaaga

SEQ ID NO: 466 - DNA, 17 bp, Homo sapiens
ccagtcgtcataaagaa

SEQ ID NO: 467 - DNA, 17 bp, Homo sapiens
cagtcgtcataaagaaa

SEQ ID NO: 468 - DNA, 17 bp, Homo sapiens
agtcgtcataaagaaaa

SEQ ID NO: 469 - DNA, 17 bp, Homo sapiens
gtcgtcataaagaaaac

SEQ ID NO: 470 - DNA, 17 bp, Homo sapiens
tcgtcataaagaaaacc

SEQ ID NO: 471 - DNA, 17 bp, Homo sapiens
cgtcataaagaaaacca

SEQ ID NO: 472 - DNA, 17 bp, Homo sapiens
gtcataaagaaaaccag

SEQ ID NO: 473 - DNA, 17 bp, Homo sapiens
tcataaagaaaaccagt

SEQ ID NO: 474 - DNA, 17 bp, Homo sapiens
cataaagaaaaccagtg

SEQ ID NO: 475 - DNA, 17 bp, Homo sapiens
ataaagaaaaccagtgc

SEQ ID NO: 476 - DNA, 17 bp, Homo sapiens
taaagaaaaccagtgcc

SEQ ID NO: 477 - DNA, 17 bp, Homo sapiens
aaagaaaaccagtgcca

SEQ ID NO: 478 - DNA, 17 bp, Homo sapiens
aagaaaaccagtgccac

SEQ ID NO: 479 - DNA, 17 bp, Homo sapiens
agaaaaccagtgccact

SEQ ID NO: 480 - DNA, 17 bp, Homo sapiens
gaaaaccagtgccactt

SEQ ID NO: 481 - DNA, 17 bp, Homo sapiens
aaaaccagtgccacttc

SEQ ID NO: 482 - DNA, 17 bp, Homo sapiens
aaaccagtgccacttca

SEQ ID NO: 483 - DNA, 17 bp, Homo sapiens
aaccagtgccacttcag

SEQ ID NO: 484 - DNA, 17 bp, Homo sapiens
accagtgccacttcagt

SEQ ID NO: 485 - DNA, 17 bp, Homo sapiens
ccagtgccacttcagtc

SEQ ID NO: 486 - DNA, 17 bp, Homo sapiens
cagtgccacttcagtca

SEQ ID NO: 487 - DNA, 17 bp, Homo sapiens
agtgccacttcagtcaa

SEQ ID NO: 488 - DNA, 17 bp, Homo sapiens
gtgccacttcagtcaac

SEQ ID NO: 489 - DNA, 17 bp, Homo sapiens
tgccacttcagtcaacc

SEQ ID NO: 490 - DNA, 17 bp, Homo sapiens
gccacttcagtcaacct

SEQ ID NO: 491 - DNA, 17 bp, Homo sapiens
ccacttcagtcaacctc

SEQ ID NO: 492 - DNA, 17 bp, Homo sapiens
cacttcagtcaacctct

SEQ ID NO: 493 - DNA, 17 bp, Homo sapiens
acttcagtcaacctctc

SEQ ID NO: 494 - DNA, 17 bp, Homo sapiens
cttcagtcaacctctcc

SEQ ID NO: 495 - DNA, 17 bp, Homo sapiens
ttcagtcaacctctcct

SEQ ID NO: 496 - DNA, 17 bp, Homo sapiens
tcagtcaacctctcctt

SEQ ID NO: 497 - DNA, 17 bp, Homo sapiens
cagtcaacctctccttg

SEQ ID NO: 498 - DNA, 17 bp, Homo sapiens
agtcaacctctccttgc

SEQ ID NO: 499 - DNA, 17 bp, Homo sapiens
gtcaacctctccttgct

SEQ ID NO: 500 - DNA, 17 bp, Homo sapiens
tcaacctctccttgctc

SEQ ID NO: 501 - DNA, 17 bp, Homo sapiens
caacctctccttgctca

SEQ ID NO: 502 - DNA, 25 bp, Homo sapiens
ttcctgctgatccggtacaaggctg

SEQ ID NO: 503 - DNA, 25 bp, Homo sapiens
tcctgctgatccggtacaaggctgt

SEQ ID NO: 504 - DNA, 25 bp, Homo sapiens
cctgctgatccggtacaaggctgtg

SEQ ID NO: 505 - DNA, 25 bp, Homo sapiens
ctgctgatccggtacaaggctgtgc

SEQ ID NO: 506 - DNA, 25 bp, Homo sapiens
tgctgatccggtacaaggctgtgca

SEQ ID NO: 507 - DNA, 25 bp, Homo sapiens
gctgatccggtacaaggctgtgcat

SEQ ID NO: 508 - DNA, 25 bp, Homo sapiens
ctgatccggtacaaggctgtgcatt

SEQ ID NO: 509 - DNA, 25 bp, Homo sapiens
tgatccggtacaaggctgtgcattt

SEQ ID NO: 510 - DNA, 25 bp, Homo sapiens
gatccggtacaaggctgtgcatttc

SEQ ID NO: 511 - DNA, 25 bp, Homo sapiens
atccggtacaaggctgtgcatttca

SEQ ID NO: 512 - DNA, 25 bp, Homo sapiens
tccggtacaaggctgtgcatttcat

SEQ ID NO: 513 - DNA, 25 bp, Homo sapiens
ccggtacaaggctgtgcatttcatc

SEQ ID NO: 514 - DNA, 25 bp, Homo sapiens
cggtacaaggctgtgcatttcatcg

SEQ ID NO: 515 - DNA, 25 bp, Homo sapiens
ggtacaaggctgtgcatttcatcgg

SEQ ID NO: 516 - DNA, 25 bp, Homo sapiens
gtacaaggctgtgcatttcatcggc

SEQ ID NO: 517 - DNA, 25 bp, Homo sapiens
tacaaggctgtgcatttcatcggca

SEQ ID NO: 518 - DNA, 25 bp, Homo sapiens
acaaggctgtgcatttcatcggcat

SEQ ID NO: 519 - DNA, 25 bp, Homo sapiens
caaggctgtgcatttcatcggcatc

SEQ ID NO: 520 - DNA, 25 bp, Homo sapiens
aaggctgtgcatttcatcggcatcg

SEQ ID NO: 521 - DNA, 25 bp, Homo sapiens
aggctgtgcatttcatcggcatcgt

SEQ ID NO: 522 - DNA, 25 bp, Homo sapiens
ggctgtgcatttcatcggcatcgtt

SEQ ID NO: 523 - DNA, 25 bp, Homo sapiens
gctgtgcatttcatcggcatcgttg

SEQ ID NO: 524 - DNA, 25 bp, Homo sapiens
ctgtgcatttcatcggcatcgttgt

SEQ ID NO: 525 - DNA, 25 bp, Homo sapiens
tgtgcatttcatcggcatcgttgtc

SEQ ID NO: 526 - DNA, 25 bp, Homo sapiens
gtgcatttcatcggcatcgttgtct

SEQ ID NO: 527 - DNA, 25 bp, Homo sapiens
tgcatttcatcggcatcgttgtctg

SEQ ID NO: 528 - DNA, 25 bp, Homo sapiens
gcatttcatcggcatcgttgtctgc

SEQ ID NO: 529 - DNA, 25 bp, Homo sapiens
catttcatcggcatcgttgtctgca

SEQ ID NO: 530 - DNA, 25 bp, Homo sapiens
atttcatcggcatcgttgtctgcat

SEQ ID NO: 531 - DNA, 25 bp, Homo sapiens
tttcatcggcatcgttgtctgcatc

SEQ ID NO: 532 - DNA, 25 bp, Homo sapiens
ttcatcggcatcgttgtctgcatcc

SEQ ID NO: 533 - DNA, 25 bp, Homo sapiens
tcatcggcatcgttgtctgcatcct

SEQ ID NO: 534 - DNA, 25 bp, Homo sapiens
catcggcatcgttgtctgcatcctg

SEQ ID NO: 535 - DNA, 25 bp, Homo sapiens
atcggcatcgttgtctgcatcctgg

SEQ ID NO: 536 - DNA, 25 bp, Homo sapiens
tcggcatcgttgtctgcatcctggg

SEQ ID NO: 537 - DNA, 25 bp, Homo sapiens
cggcatcgttgtctgcatcctggga

SEQ ID NO: 538 - DNA, 25 bp, Homo sapiens
ggcatcgttgtctgcatcctgggaa

SEQ ID NO: 539 - DNA, 25 bp, Homo sapiens
gcatcgttgtctgcatcctgggaat

SEQ ID NO: 540 - DNA, 25 bp, Homo sapiens
catcgttgtctgcatcctgggaatg

SEQ ID NO: 541 - DNA, 25 bp, Homo sapiens
atcgttgtctgcatcctgggaatgg

SEQ ID NO: 542 - DNA, 25 bp, Homo sapiens
tcgttgtctgcatcctgggaatggg

SEQ ID NO: 543 - DNA, 25 bp, Homo sapiens
cgttgtctgcatcctgggaatgggc

SEQ ID NO: 544 - DNA, 25 bp, Homo sapiens
gttgtctgcatcctgggaatgggct

SEQ ID NO: 545 - DNA, 25 bp, Homo sapiens
ttgtctgcatcctgggaatgggctg

SEQ ID NO: 546 - DNA, 25 bp, Homo sapiens
tgtctgcatcctgggaatgggctgc

SEQ ID NO: 547 - DNA, 25 bp, Homo sapiens
gtctgcatcctgggaatgggctgca

SEQ ID NO: 548 - DNA, 25 bp, Homo sapiens
tctgcatcctgggaatgggctgcat

SEQ ID NO: 549 - DNA, 25 bp, Homo sapiens
ctgcatcctgggaatgggctgcatg

SEQ ID NO: 550 - DNA, 25 bp, Homo sapiens
tgcatcctgggaatgggctgcatgg

SEQ ID NO: 551 - DNA, 25 bp, Homo sapiens
gcatcctgggaatgggctgcatggt

SEQ ID NO: 552 - DNA, 25 bp, Homo sapiens
catcctgggaatgggctgcatggtg

SEQ ID NO: 553 - DNA, 25 bp, Homo sapiens
atcctgggaatgggctgcatggtgg

SEQ ID NO: 554 - DNA, 25 bp, Homo sapiens
tcctgggaatgggctgcatggtggg

SEQ ID NO: 555 - DNA, 25 bp, Homo sapiens
cctgggaatgggctgcatggtggga

SEQ ID NO: 556 - DNA, 25 bp, Homo sapiens
ctgggaatgggctgcatggtgggag

SEQ ID NO: 557 - DNA, 25 bp, Homo sapiens
tgggaatgggctgcatggtgggagc

SEQ ID NO: 558 - DNA, 25 bp, Homo sapiens
gggaatgggctgcatggtgggagca

SEQ ID NO: 559 - DNA, 25 bp, Homo sapiens
ggaatgggctgcatggtgggagcag

SEQ ID NO: 560 - DNA, 25 bp, Homo sapiens
gaatgggctgcatggtgggagcaga

SEQ ID NO: 561 - DNA, 25 bp, Homo sapiens
aatgggctgcatggtgggagcagat

SEQ ID NO: 562 - DNA, 25 bp, Homo sapiens
atgggctgcatggtgggagcagatg

SEQ ID NO: 563 - DNA, 25 bp, Homo sapiens
tgggctgcatggtgggagcagatgt

SEQ ID NO: 564 - DNA, 25 bp, Homo sapiens
gggctgcatggtgggagcagatgtg

SEQ ID NO: 565 - DNA, 25 bp, Homo sapiens
ggctgcatggtgggagcagatgtgc

SEQ ID NO: 566 - DNA, 25 bp, Homo sapiens
gctgcatggtgggagcagatgtgct

SEQ ID NO: 567 - DNA, 25 bp, Homo sapiens
ctgcatggtgggagcagatgtgctt

SEQ ID NO: 568 - DNA, 25 bp, Homo sapiens
tgcatggtgggagcagatgtgcttg

SEQ ID NO: 569 - DNA, 25 bp, Homo sapiens
gcatggtgggagcagatgtgcttgt

SEQ ID NO: 570 - DNA, 25 bp, Homo sapiens
catggtgggagcagatgtgcttgtg

SEQ ID NO: 571 - DNA, 25 bp, Homo sapiens
atggtgggagcagatgtgcttgtgg

SEQ ID NO: 572 - DNA, 25 bp, Homo sapiens
tggtgggagcagatgtgcttgtggg

SEQ ID NO: 573 - DNA, 25 bp, Homo sapiens
ggtgggagcagatgtgcttgtggga

SEQ ID NO: 574 - DNA, 25 bp, Homo sapiens
gtgggagcagatgtgcttgtgggaa

SEQ ID NO: 575 - DNA, 25 bp, Homo sapiens
tgggagcagatgtgcttgtgggaag

SEQ ID NO: 576 - DNA, 25 bp, Homo sapiens
gggagcagatgtgcttgtgggaaga

SEQ ID NO: 577 - DNA, 25 bp, Homo sapiens
ggagcagatgtgcttgtgggaagac

SEQ ID NO: 578 - DNA, 25 bp, Homo sapiens
gagcagatgtgcttgtgggaagaca

SEQ ID NO: 579 - DNA, 25 bp, Homo sapiens
agcagatgtgcttgtgggaagacat

SEQ ID NO: 580 - DNA, 25 bp, Homo sapiens
gcagatgtgcttgtgggaagacatc

SEQ ID NO: 581 - DNA, 25 bp, Homo sapiens
cagatgtgcttgtgggaagacatca

SEQ ID NO: 582 - DNA, 25 bp, Homo sapiens
agatgtgcttgtgggaagacatcag

SEQ ID NO: 583 - DNA, 25 bp, Homo sapiens
gatgtgcttgtgggaagacatcagg

SEQ ID NO: 584 - DNA, 25 bp, Homo sapiens
atgtgcttgtgggaagacatcaggg

SEQ ID NO: 585 - DNA, 25 bp, Homo sapiens
tgtgcttgtgggaagacatcaggga

SEQ ID NO: 586 - DNA, 25 bp, Homo sapiens
gtgcttgtgggaagacatcagggag

SEQ ID NO: 587 - DNA, 25 bp, Homo sapiens
tgcttgtgggaagacatcagggagc

SEQ ID NO: 588 - DNA, 25 bp, Homo sapiens
gcttgtgggaagacatcagggagca

SEQ ID NO: 589 - DNA, 25 bp, Homo sapiens
cttgtgggaagacatcagggagcag

SEQ ID NO: 590 - DNA, 25 bp, Homo sapiens
ttgtgggaagacatcagggagcagg

SEQ ID NO: 591 - DNA, 25 bp, Homo sapiens
tgtgggaagacatcagggagcaggg

SEQ ID NO: 592 - DNA, 25 bp, Homo sapiens
gtgggaagacatcagggagcagggg

SEQ ID NO: 593 - DNA, 25 bp, Homo sapiens
tgggaagacatcagggagcagggga

SEQ ID NO: 594 - DNA, 25 bp, Homo sapiens
gggaagacatcagggagcaggggaa

SEQ ID NO: 595 - DNA, 25 bp, Homo sapiens
ggaagacatcagggagcaggggaaa

SEQ ID NO: 596 - DNA, 25 bp, Homo sapiens
gaagacatcagggagcaggggaaaa

SEQ ID NO: 597 - DNA, 25 bp, Homo sapiens
aagacatcagggagcaggggaaaat

SEQ ID NO: 598 - DNA, 25 bp, Homo sapiens
agacatcagggagcaggggaaaata

SEQ ID NO: 599 - DNA, 25 bp, Homo sapiens
gacatcagggagcaggggaaaataa

SEQ ID NO: 600 - DNA, 25 bp, Homo sapiens
acatcagggagcaggggaaaataag

SEQ ID NO: 601 - DNA, 25 bp, Homo sapiens
catcagggagcaggggaaaataagc

SEQ ID NO: 602 - DNA, 25 bp, Homo sapiens
atcagggagcaggggaaaataagct

SEQ ID NO: 603 - DNA, 25 bp, Homo sapiens
tcagggagcaggggaaaataagctg

SEQ ID NO: 604 - DNA, 25 bp, Homo sapiens
cagggagcaggggaaaataagctgg

SEQ ID NO: 605 - DNA, 25 bp, Homo sapiens
agggagcaggggaaaataagctggt

SEQ ID NO: 606 - DNA, 25 bp, Homo sapiens
gggagcaggggaaaataagctggta

SEQ ID NO: 607 - DNA, 25 bp, Homo sapiens
ggagcaggggaaaataagctggtag

SEQ ID NO: 608 - DNA, 25 bp, Homo sapiens
gagcaggggaaaataagctggtagg

SEQ ID NO: 609 - DNA, 25 bp, Homo sapiens
agcaggggaaaataagctggtaggg

SEQ ID NO: 610 - DNA, 25 bp, Homo sapiens
gcaggggaaaataagctggtagggg

SEQ ID NO: 611 - DNA, 25 bp, Homo sapiens
caggggaaaataagctggtagggga

SEQ ID NO: 612 - DNA, 25 bp, Homo sapiens
aggggaaaataagctggtaggggac

SEQ ID NO: 613 - DNA, 25 bp, Homo sapiens
ggggaaaataagctggtaggggacc

SEQ ID NO: 614 - DNA, 25 bp, Homo sapiens
gggaaaataagctggtaggggacct

SEQ ID NO: 615 - DNA, 25 bp, Homo sapiens
ggaaaataagctggtaggggacctt

SEQ ID NO: 616 - DNA, 25 bp, Homo sapiens
gaaaataagctggtaggggaccttc

SEQ ID NO: 617 - DNA, 25 bp, Homo sapiens
aaaataagctggtaggggaccttct

SEQ ID NO: 618 - DNA, 25 bp, Homo sapiens
aaataagctggtaggggaccttctg

SEQ ID NO: 619 - DNA, 25 bp, Homo sapiens
aataagctggtaggggaccttctgg

SEQ ID NO: 620 - DNA, 25 bp, Homo sapiens
ataagctggtaggggaccttctggt

SEQ ID NO: 621 - DNA, 25 bp, Homo sapiens
taagctggtaggggaccttctggtc

SEQ ID NO: 622 - DNA, 25 bp, Homo sapiens
aagctggtaggggaccttctggtct

SEQ ID NO: 623 - DNA, 25 bp, Homo sapiens
agctggtaggggaccttctggtctt

SEQ ID NO: 624 - DNA, 25 bp, Homo sapiens
gctggtaggggaccttctggtctta

SEQ ID NO: 625 - DNA, 25 bp, Homo sapiens
ctggtaggggaccttctggtcttag

SEQ ID NO: 626 - DNA, 25 bp, Homo sapiens
tggtaggggaccttctggtcttagg

SEQ ID NO: 627 - DNA, 25 bp, Homo sapiens
ggtaggggaccttctggtcttagga

SEQ ID NO: 628 - DNA, 25 bp, Homo sapiens
gtaggggaccttctggtcttaggag

SEQ ID NO: 629 - DNA, 25 bp, Homo sapiens
taggggaccttctggtcttaggagg

SEQ ID NO: 630 - DNA, 25 bp, Homo sapiens
aggggaccttctggtcttaggagga

SEQ ID NO: 631 - DNA, 25 bp, Homo sapiens
ggggaccttctggtcttaggaggag

SEQ ID NO: 632 - DNA, 25 bp, Homo sapiens
gggaccttctggtcttaggaggagc

SEQ ID NO: 633 - DNA, 25 bp, Homo sapiens
ggaccttctggtcttaggaggagcc

SEQ ID NO: 634 - DNA, 25 bp, Homo sapiens
gaccttctggtcttaggaggagcca

SEQ ID NO: 635 - DNA, 25 bp, Homo sapiens
accttctggtcttaggaggagccac

SEQ ID NO: 636 - DNA, 25 bp, Homo sapiens
ccttctggtcttaggaggagccaca

SEQ ID NO: 637 - DNA, 25 bp, Homo sapiens
cttctggtcttaggaggagccacac

SEQ ID NO: 638 - DNA, 25 bp, Homo sapiens
ttctggtcttaggaggagccacact

SEQ ID NO: 639 - DNA, 25 bp, Homo sapiens
tctggtcttaggaggagccacactc

SEQ ID NO: 640 - DNA, 25 bp, Homo sapiens
ctggtcttaggaggagccacactct

SEQ ID NO: 641 - DNA, 25 bp, Homo sapiens
tggtcttaggaggagccacactcta

SEQ ID NO: 642 - DNA, 25 bp, Homo sapiens
ggtcttaggaggagccacactctat

SEQ ID NO: 643 - DNA, 25 bp, Homo sapiens
gtcttaggaggagccacactctatg

SEQ ID NO: 644 - DNA, 25 bp, Homo sapiens
tcttaggaggagccacactctatgg

SEQ ID NO: 645 - DNA, 25 bp, Homo sapiens
cttaggaggagccacactctatggt

SEQ ID NO: 646 - DNA, 25 bp, Homo sapiens
ttaggaggagccacactctatggta

SEQ ID NO: 647 - DNA, 25 bp, Homo sapiens
taggaggagccacactctatggtat

SEQ ID NO: 648 - DNA, 25 bp, Homo sapiens
aggaggagccacactctatggtatt

SEQ ID NO: 649 - DNA, 25 bp, Homo sapiens
ggaggagccacactctatggtattt

SEQ ID NO: 650 - DNA, 25 bp, Homo sapiens
gaggagccacactctatggtatttc

SEQ ID NO: 651 - DNA, 25 bp, Homo sapiens
aggagccacactctatggtatttct

SEQ ID NO: 652 - DNA, 25 bp, Homo sapiens
ggagccacactctatggtatttcta

SEQ ID NO: 653 - DNA, 25 bp, Homo sapiens
gagccacactctatggtatttctaa

SEQ ID NO: 654 - DNA, 25 bp, Homo sapiens
agccacactctatggtatttctaac

SEQ ID NO: 655 - DNA, 25 bp, Homo sapiens
gccacactctatggtatttctaacg

SEQ ID NO: 656 - DNA, 25 bp, Homo sapiens
ccacactctatggtatttctaacgt

SEQ ID NO: 657 - DNA, 25 bp, Homo sapiens
cacactctatggtatttctaacgtc

SEQ ID NO: 658 - DNA, 25 bp, Homo sapiens
acactctatggtatttctaacgtct

SEQ ID NO: 659 - DNA, 25 bp, Homo sapiens
cactctatggtatttctaacgtctg

SEQ ID NO: 660 - DNA, 25 bp, Homo sapiens
actctatggtatttctaacgtctgg

SEQ ID NO: 661 - DNA, 25 bp, Homo sapiens
ctctatggtatttctaacgtctggg

SEQ ID NO: 662 - DNA, 25 bp, Homo sapiens
tctatggtatttctaacgtctggga

SEQ ID NO: 663 - DNA, 25 bp, Homo sapiens
ctatggtatttctaacgtctgggaa

SEQ ID NO: 664 - DNA, 25 bp, Homo sapiens
tatggtatttctaacgtctgggaag

SEQ ID NO: 665 - DNA, 25 bp, Homo sapiens
atggtatttctaacgtctgggaaga

SEQ ID NO: 666 - DNA, 25 bp, Homo sapiens
tggtatttctaacgtctgggaagaa

SEQ ID NO: 667 - DNA, 25 bp, Homo sapiens
ggtatttctaacgtctgggaagaat

SEQ ID NO: 668 - DNA, 25 bp, Homo sapiens
gtatttctaacgtctgggaagaata

SEQ ID NO: 669 - DNA, 25 bp, Homo sapiens
tatttctaacgtctgggaagaatac

SEQ ID NO: 670 - DNA, 25 bp, Homo sapiens
atttctaacgtctgggaagaataca

SEQ ID NO: 671 - DNA, 25 bp, Homo sapiens
tttctaacgtctgggaagaatacat

SEQ ID NO: 672 - DNA, 25 bp, Homo sapiens
ttctaacgtctgggaagaatacatc

SEQ ID NO: 673 - DNA, 25 bp, Homo sapiens
tctaacgtctgggaagaatacatca

SEQ ID NO: 674 - DNA, 25 bp, Homo sapiens
ctaacgtctgggaagaatacatcat

SEQ ID NO: 675 - DNA, 25 bp, Homo sapiens
taacgtctgggaagaatacatcatc

SEQ ID NO: 676 - DNA, 25 bp, Homo sapiens
aacgtctgggaagaatacatcatcc

SEQ ID NO: 677 - DNA, 25 bp, Homo sapiens
acgtctgggaagaatacatcatccg

SEQ ID NO: 678 - DNA, 25 bp, Homo sapiens
cgtctgggaagaatacatcatccga

SEQ ID NO: 679 - DNA, 25 bp, Homo sapiens
gtctgggaagaatacatcatccgaa

SEQ ID NO: 680 - DNA, 25 bp, Homo sapiens
tctgggaagaatacatcatccgaac

SEQ ID NO: 681 - DNA, 25 bp, Homo sapiens
ctgggaagaatacatcatccgaact

SEQ ID NO: 682 - DNA, 25 bp, Homo sapiens
tgggaagaatacatcatccgaactc

SEQ ID NO: 683 - DNA, 25 bp, Homo sapiens
gggaagaatacatcatccgaactct

SEQ ID NO: 684 - DNA, 25 bp, Homo sapiens
ggaagaatacatcatccgaactctg

SEQ ID NO: 685 - DNA, 25 bp, Homo sapiens
gaagaatacatcatccgaactctga

SEQ ID NO: 686 - DNA, 25 bp, Homo sapiens
aagaatacatcatccgaactctgag

SEQ ID NO: 687 - DNA, 25 bp, Homo sapiens
agaatacatcatccgaactctgagc

SEQ ID NO: 688 - DNA, 25 bp, Homo sapiens
gaatacatcatccgaactctgagcc

SEQ ID NO: 689 - DNA, 25 bp, Homo sapiens
aatacatcatccgaactctgagccg

SEQ ID NO: 690 - DNA, 25 bp, Homo sapiens
atacatcatccgaactctgagccga

SEQ ID NO: 691 - DNA, 25 bp, Homo sapiens
tacatcatccgaactctgagccgag

SEQ ID NO: 692 - DNA, 25 bp, Homo sapiens
acatcatccgaactctgagccgagt

SEQ ID NO: 693 - DNA, 25 bp, Homo sapiens
catcatccgaactctgagccgagtg

SEQ ID NO: 694 - DNA, 25 bp, Homo sapiens
atcatccgaactctgagccgagtgg

SEQ ID NO: 695 - DNA, 25 bp, Homo sapiens
tcatccgaactctgagccgagtgga

SEQ ID NO: 696 - DNA, 25 bp, Homo sapiens
catccgaactctgagccgagtggaa

SEQ ID NO: 697 - DNA, 25 bp, Homo sapiens
atccgaactctgagccgagtggaat

SEQ ID NO: 698 - DNA, 25 bp, Homo sapiens
tccgaactctgagccgagtggaatt

SEQ ID NO: 699 - DNA, 25 bp, Homo sapiens
ccgaactctgagccgagtggaattc

SEQ ID NO: 700 - DNA, 25 bp, Homo sapiens
cgaactctgagccgagtggaattcc

SEQ ID NO: 701 - DNA, 25 bp, Homo sapiens
gaactctgagccgagtggaattcct

SEQ ID NO: 702 - DNA, 25 bp, Homo sapiens
aactctgagccgagtggaattcctg

SEQ ID NO: 703 - DNA, 25 bp, Homo sapiens
actctgagccgagtggaattcctgg

SEQ ID NO: 704 - DNA, 25 bp, Homo sapiens
ctctgagccgagtggaattcctggg

SEQ ID NO: 705 - DNA, 25 bp, Homo sapiens
tctgagccgagtggaattcctggga

SEQ ID NO: 706 - DNA, 25 bp, Homo sapiens
ctgagccgagtggaattcctgggaa

SEQ ID NO: 707 - DNA, 25 bp, Homo sapiens
tgagccgagtggaattcctgggaat

SEQ ID NO: 708 - DNA, 25 bp, Homo sapiens
gagccgagtggaattcctgggaatg

SEQ ID NO: 709 - DNA, 25 bp, Homo sapiens
agccgagtggaattcctgggaatga

SEQ ID NO: 710 - DNA, 25 bp, Homo sapiens
gccgagtggaattcctgggaatgat

SEQ ID NO: 711 - DNA, 25 bp, Homo sapiens
ccgagtggaattcctgggaatgatt

SEQ ID NO: 712 - DNA, 25 bp, Homo sapiens
cgagtggaattcctgggaatgattg

SEQ ID NO: 713 - DNA, 25 bp, Homo sapiens
gagtggaattcctgggaatgattgg

SEQ ID NO: 714 - DNA, 25 bp, Homo sapiens
agtggaattcctgggaatgattggt

SEQ ID NO: 715 - DNA, 25 bp, Homo sapiens
gtggaattcctgggaatgattggtc

SEQ ID NO: 716 - DNA, 25 bp, Homo sapiens
tggaattcctgggaatgattggtct

SEQ ID NO: 717 - DNA, 25 bp, Homo sapiens
ggaattcctgggaatgattggtctc

SEQ ID NO: 718 - DNA, 25 bp, Homo sapiens
gaattcctgggaatgattggtctct

SEQ ID NO: 719 - DNA, 25 bp, Homo sapiens
aattcctgggaatgattggtctctt

SEQ ID NO: 720 - DNA, 25 bp, Homo sapiens
attcctgggaatgattggtctcttt

SEQ ID NO: 721 - DNA, 25 bp, Homo sapiens
ttcctgggaatgattggtctctttg

SEQ ID NO: 722 - DNA, 25 bp, Homo sapiens
tcctgggaatgattggtctctttgg

SEQ ID NO: 723 - DNA, 25 bp, Homo sapiens
cctgggaatgattggtctctttgga

SEQ ID NO: 724 - DNA, 25 bp, Homo sapiens
ctgggaatgattggtctctttggag

SEQ ID NO: 725 - DNA, 25 bp, Homo sapiens
tgggaatgattggtctctttggagc

SEQ ID NO: 726 - DNA, 25 bp, Homo sapiens
gggaatgattggtctctttggagca

SEQ ID NO: 727 - DNA, 25 bp, Homo sapiens
ggaatgattggtctctttggagcat

SEQ ID NO: 728 - DNA, 25 bp, Homo sapiens
gaatgattggtctctttggagcatt

SEQ ID NO: 729 - DNA, 25 bp, Homo sapiens
aatgattggtctctttggagcattt

SEQ ID NO: 730 - DNA, 25 bp, Homo sapiens
atgattggtctctttggagcatttt

SEQ ID NO: 731 - DNA, 25 bp, Homo sapiens
tgattggtctctttggagcattttt

SEQ ID NO: 732 - DNA, 25 bp, Homo sapiens
gattggtctctttggagcatttttc

SEQ ID NO: 733 - DNA, 25 bp, Homo sapiens
attggtctctttggagcatttttca

SEQ ID NO: 734 - DNA, 25 bp, Homo sapiens
ttggtctctttggagcatttttcag

SEQ ID NO: 735 - DNA, 25 bp, Homo sapiens
tggtctctttggagcatttttcagt

SEQ ID NO: 736 - DNA, 25 bp, Homo sapiens
ggtctctttggagcatttttcagtg

SEQ ID NO: 737 - DNA, 25 bp, Homo sapiens
gtctctttggagcatttttcagtgg

SEQ ID NO: 738 - DNA, 25 bp, Homo sapiens
tctctttggagcatttttcagtgga

SEQ ID NO: 739 - DNA, 25 bp, Homo sapiens
ctctttggagcatttttcagtggaa

SEQ ID NO: 740 - DNA, 25 bp, Homo sapiens
tctttggagcatttttcagtggaat

SEQ ID NO: 741 - DNA, 25 bp, Homo sapiens
ctttggagcatttttcagtggaatt

SEQ ID NO: 742 - DNA, 25 bp, Homo sapiens
tttggagcatttttcagtggaattc

SEQ ID NO: 743 - DNA, 25 bp, Homo sapiens
ttggagcatttttcagtggaattca

SEQ ID NO: 744 - DNA, 25 bp, Homo sapiens
tggagcatttttcagtggaattcaa

SEQ ID NO: 745 - DNA, 25 bp, Homo sapiens
ggagcatttttcagtggaattcaat

SEQ ID NO: 746 - DNA, 25 bp, Homo sapiens
gagcatttttcagtggaattcaatt

SEQ ID NO: 747 - DNA, 25 bp, Homo sapiens
agcatttttcagtggaattcaatta

SEQ ID NO: 748 - DNA, 25 bp, Homo sapiens
gcatttttcagtggaattcaattag

SEQ ID NO: 749 - DNA, 25 bp, Homo sapiens
catttttcagtggaattcaattagc

SEQ ID NO: 750 - DNA, 25 bp, Homo sapiens
atttttcagtggaattcaattagct

SEQ ID NO: 751 - DNA, 25 bp, Homo sapiens
tttttcagtggaattcaattagcta

SEQ ID NO: 752 - DNA, 25 bp, Homo sapiens
ttttcagtggaattcaattagctat

SEQ ID NO: 753 - DNA, 25 bp, Homo sapiens
tttcagtggaattcaattagctata

SEQ ID NO: 754 - DNA, 25 bp, Homo sapiens
ttcagtggaattcaattagctataa

SEQ ID NO: 755 - DNA, 25 bp, Homo sapiens
tcagtggaattcaattagctataat

SEQ ID NO: 756 - DNA, 25 bp, Homo sapiens
cagtggaattcaattagctataatg

SEQ ID NO: 757 - DNA, 25 bp, Homo sapiens
agtggaattcaattagctataatgg

SEQ ID NO: 758 - DNA, 25 bp, Homo sapiens
gtggaattcaattagctataatgga

SEQ ID NO: 759 - DNA, 25 bp, Homo sapiens
tggaattcaattagctataatggag

SEQ ID NO: 760 - DNA, 25 bp, Homo sapiens
ggaattcaattagctataatggagc

SEQ ID NO: 761 - DNA, 25 bp, Homo sapiens
gaattcaattagctataatggagca

SEQ ID NO: 762 - DNA, 25 bp, Homo sapiens
aattcaattagctataatggagcat

SEQ ID NO: 763 - DNA, 25 bp, Homo sapiens
attcaattagctataatggagcata

SEQ ID NO: 764 - DNA, 25 bp, Homo sapiens
ttcaattagctataatggagcataa

SEQ ID NO: 765 - DNA, 25 bp, Homo sapiens
tcaattagctataatggagcataag

SEQ ID NO: 766 - DNA, 25 bp, Homo sapiens
caattagctataatggagcataagg

SEQ ID NO: 767 - DNA, 25 bp, Homo sapiens
aattagctataatggagcataagga

SEQ ID NO: 768 - DNA, 25 bp, Homo sapiens
attagctataatggagcataaggaa

SEQ ID NO: 769 - DNA, 25 bp, Homo sapiens
ttagctataatggagcataaggaac

SEQ ID NO: 770 - DNA, 25 bp, Homo sapiens
tagctataatggagcataaggaact

SEQ ID NO: 771 - DNA, 25 bp, Homo sapiens
agctataatggagcataaggaactg

SEQ ID NO: 772 - DNA, 25 bp, Homo sapiens
gctataatggagcataaggaactgt

SEQ ID NO: 773 - DNA, 25 bp, Homo sapiens
ctataatggagcataaggaactgtt

SEQ ID NO: 774 - DNA, 25 bp, Homo sapiens
tataatggagcataaggaactgttg

SEQ ID NO: 775 - DNA, 25 bp, Homo sapiens
ataatggagcataaggaactgttga

SEQ ID NO: 776 - DNA, 25 bp, Homo sapiens
taatggagcataaggaactgttgaa

SEQ ID NO: 777 - DNA, 25 bp, Homo sapiens
aatggagcataaggaactgttgaag

SEQ ID NO: 778 - DNA, 25 bp, Homo sapiens
atggagcataaggaactgttgaagg

SEQ ID NO: 779 - DNA, 25 bp, Homo sapiens
tggagcataaggaactgttgaaggt

SEQ ID NO: 780 - DNA, 25 bp, Homo sapiens
ggagcataaggaactgttgaaggtg

SEQ ID NO: 781 - DNA, 25 bp, Homo sapiens
gagcataaggaactgttgaaggtgc

SEQ ID NO: 782 - DNA, 25 bp, Homo sapiens
agcataaggaactgttgaaggtgcc

SEQ ID NO: 783 - DNA, 25 bp, Homo sapiens
gcataaggaactgttgaaggtgccc

SEQ ID NO: 784 - DNA, 25 bp, Homo sapiens
cataaggaactgttgaaggtgccct

SEQ ID NO: 785 - DNA, 25 bp, Homo sapiens
ataaggaactgttgaaggtgccctg

SEQ ID NO: 786 - DNA, 25 bp, Homo sapiens
taaggaactgttgaaggtgccctgg

SEQ ID NO: 787 - DNA, 25 bp, Homo sapiens
aaggaactgttgaaggtgccctggg

SEQ ID NO: 788 - DNA, 25 bp, Homo sapiens
aggaactgttgaaggtgccctggga

SEQ ID NO: 789 - DNA, 25 bp, Homo sapiens
ggaactgttgaaggtgccctgggac

SEQ ID NO: 790 - DNA, 25 bp, Homo sapiens
gaactgttgaaggtgccctgggact

SEQ ID NO: 791 - DNA, 25 bp, Homo sapiens
aactgttgaaggtgccctgggactg

SEQ ID NO: 792 - DNA, 25 bp, Homo sapiens
actgttgaaggtgccctgggactgg

SEQ ID NO: 793 - DNA, 25 bp, Homo sapiens
ctgttgaaggtgccctgggactggc

SEQ ID NO: 794 - DNA, 25 bp, Homo sapiens
tgttgaaggtgccctgggactggca

SEQ ID NO: 795 - DNA, 25 bp, Homo sapiens
gttgaaggtgccctgggactggcaa

SEQ ID NO: 796 - DNA, 25 bp, Homo sapiens
ttgaaggtgccctgggactggcaaa

SEQ ID NO: 797 - DNA, 25 bp, Homo sapiens
tgaaggtgccctgggactggcaaat

SEQ ID NO: 798 - DNA, 25 bp, Homo sapiens
gaaggtgccctgggactggcaaata

SEQ ID NO: 799 - DNA, 25 bp, Homo sapiens
aaggtgccctgggactggcaaatag

SEQ ID NO: 800 - DNA, 25 bp, Homo sapiens
aggtgccctgggactggcaaatagg

SEQ ID NO: 801 - DNA, 25 bp, Homo sapiens
ggtgccctgggactggcaaatagga

SEQ ID NO: 802 - DNA, 25 bp, Homo sapiens
gtgccctgggactggcaaataggac

SEQ ID NO: 803 - DNA, 25 bp, Homo sapiens
tgccctgggactggcaaataggact

SEQ ID NO: 804 - DNA, 25 bp, Homo sapiens
gccctgggactggcaaataggactg

SEQ ID NO: 805 - DNA, 25 bp, Homo sapiens
ccctgggactggcaaataggactgc

SEQ ID NO: 806 - DNA, 25 bp, Homo sapiens
cctgggactggcaaataggactgct

SEQ ID NO: 807 - DNA, 25 bp, Homo sapiens
ctgggactggcaaataggactgctc

SEQ ID NO: 808 - DNA, 25 bp, Homo sapiens
tgggactggcaaataggactgctct

SEQ ID NO: 809 - DNA, 25 bp, Homo sapiens
gggactggcaaataggactgctcta

SEQ ID NO: 810 - DNA, 25 bp, Homo sapiens
ggactggcaaataggactgctctac

SEQ ID NO: 811 - DNA, 25 bp, Homo sapiens
gactggcaaataggactgctctacg

SEQ ID NO: 812 - DNA, 25 bp, Homo sapiens
actggcaaataggactgctctacgt

SEQ ID NO: 813 - DNA, 25 bp, Homo sapiens
ctggcaaataggactgctctacgtt

SEQ ID NO: 814 - DNA, 25 bp, Homo sapiens
tggcaaataggactgctctacgttg

SEQ ID NO: 815 - DNA, 25 bp, Homo sapiens
ggcaaataggactgctctacgttgg

SEQ ID NO: 816 - DNA, 25 bp, Homo sapiens
gcaaataggactgctctacgttggc

SEQ ID NO: 817 - DNA, 25 bp, Homo sapiens
caaataggactgctctacgttggct

SEQ ID NO: 818 - DNA, 25 bp, Homo sapiens
aaataggactgctctacgttggctt

SEQ ID NO: 819 - DNA, 25 bp, Homo sapiens
aataggactgctctacgttggcttt

SEQ ID NO: 820 - DNA, 25 bp, Homo sapiens
ataggactgctctacgttggcttta

SEQ ID NO: 821 - DNA, 25 bp, Homo sapiens
taggactgctctacgttggctttag

SEQ ID NO: 822 - DNA, 25 bp, Homo sapiens
aggactgctctacgttggctttagt

SEQ ID NO: 823 - DNA, 25 bp, Homo sapiens
ggactgctctacgttggctttagtg

SEQ ID NO: 824 - DNA, 25 bp, Homo sapiens
gactgctctacgttggctttagtgc

SEQ ID NO: 825 - DNA, 25 bp, Homo sapiens
actgctctacgttggctttagtgcc

SEQ ID NO: 826 - DNA, 25 bp, Homo sapiens
ctgctctacgttggctttagtgcct

SEQ ID NO: 827 - DNA, 25 bp, Homo sapiens
tgctctacgttggctttagtgcctg

SEQ ID NO: 828 - DNA, 25 bp, Homo sapiens
gctctacgttggctttagtgcctgc

SEQ ID NO: 829 - DNA, 25 bp, Homo sapiens
ctctacgttggctttagtgcctgca

SEQ ID NO: 830 - DNA, 25 bp, Homo sapiens
tctacgttggctttagtgcctgcat

SEQ ID NO: 831 - DNA, 25 bp, Homo sapiens
ctacgttggctttagtgcctgcatg

SEQ ID NO: 832 - DNA, 25 bp, Homo sapiens
tacgttggctttagtgcctgcatgt

SEQ ID NO: 833 - DNA, 25 bp, Homo sapiens
acgttggctttagtgcctgcatgtt

SEQ ID NO: 834 - DNA, 25 bp, Homo sapiens
cgttggctttagtgcctgcatgttt

SEQ ID NO: 835 - DNA, 25 bp, Homo sapiens
gttggctttagtgcctgcatgtttg

SEQ ID NO: 836 - DNA, 25 bp, Homo sapiens
ttggctttagtgcctgcatgtttgg

SEQ ID NO: 837 - DNA, 25 bp, Homo sapiens
tggctttagtgcctgcatgtttggt

SEQ ID NO: 838 - DNA, 25 bp, Homo sapiens
ggctttagtgcctgcatgtttggtc

SEQ ID NO: 839 - DNA, 25 bp, Homo sapiens
gctttagtgcctgcatgtttggtct

SEQ ID NO: 840 - DNA, 25 bp, Homo sapiens
ctttagtgcctgcatgtttggtctc

SEQ ID NO: 841 - DNA, 25 bp, Homo sapiens
tttagtgcctgcatgtttggtctct

SEQ ID NO: 842 - DNA, 25 bp, Homo sapiens
ttagtgcctgcatgtttggtctcta

SEQ ID NO: 843 - DNA, 25 bp, Homo sapiens
tagtgcctgcatgtttggtctctac

SEQ ID NO: 844 - DNA, 25 bp, Homo sapiens
agtgcctgcatgtttggtctctaca

SEQ ID NO: 845 - DNA, 25 bp, Homo sapiens
gtgcctgcatgtttggtctctacag

SEQ ID NO: 846 - DNA, 25 bp, Homo sapiens
tgcctgcatgtttggtctctacagc

SEQ ID NO: 847 - DNA, 25 bp, Homo sapiens
gcctgcatgtttggtctctacagct

SEQ ID NO: 848 - DNA, 25 bp, Homo sapiens
cctgcatgtttggtctctacagctt

SEQ ID NO: 849 - DNA, 25 bp, Homo sapiens
ctgcatgtttggtctctacagcttt

SEQ ID NO: 850 - DNA, 25 bp, Homo sapiens
tgcatgtttggtctctacagcttta

SEQ ID NO: 851 - DNA, 25 bp, Homo sapiens
gcatgtttggtctctacagctttat

SEQ ID NO: 852 - DNA, 25 bp, Homo sapiens
catgtttggtctctacagctttatg

SEQ ID NO: 853 - DNA, 25 bp, Homo sapiens
atgtttggtctctacagctttatgc

SEQ ID NO: 854 - DNA, 25 bp, Homo sapiens
tgtttggtctctacagctttatgcc

SEQ ID NO: 855 - DNA, 25 bp, Homo sapiens
gtttggtctctacagctttatgcca

SEQ ID NO: 856 - DNA, 25 bp, Homo sapiens
tttggtctctacagctttatgccag

SEQ ID NO: 857 - DNA, 25 bp, Homo sapiens
ttggtctctacagctttatgccagt

SEQ ID NO: 858 - DNA, 25 bp, Homo sapiens
tggtctctacagctttatgccagtc

SEQ ID NO: 859 - DNA, 25 bp, Homo sapiens
ggtctctacagctttatgccagtcg

SEQ ID NO: 860 - DNA, 25 bp, Homo sapiens
gtctctacagctttatgccagtcgt

SEQ ID NO: 861 - DNA, 25 bp, Homo sapiens
tctctacagctttatgccagtcgtc

SEQ ID NO: 862 - DNA, 25 bp, Homo sapiens
ctctacagctttatgccagtcgtca

SEQ ID NO: 863 - DNA, 25 bp, Homo sapiens
tctacagctttatgccagtcgtcat

SEQ ID NO: 864 - DNA, 25 bp, Homo sapiens
ctacagctttatgccagtcgtcata

SEQ ID NO: 865 - DNA, 25 bp, Homo sapiens
tacagctttatgccagtcgtcataa

SEQ ID NO: 866 - DNA, 25 bp, Homo sapiens
acagctttatgccagtcgtcataaa

SEQ ID NO: 867 - DNA, 25 bp, Homo sapiens
cagctttatgccagtcgtcataaag

SEQ ID NO: 868 - DNA, 25 bp, Homo sapiens
agctttatgccagtcgtcataaaga

SEQ ID NO: 869 - DNA, 25 bp, Homo sapiens
gctttatgccagtcgtcataaagaa

SEQ ID NO: 870 - DNA, 25 bp, Homo sapiens
ctttatgccagtcgtcataaagaaa

SEQ ID NO: 871 - DNA, 25 bp, Homo sapiens
tttatgccagtcgtcataaagaaaa

SEQ ID NO: 872 - DNA, 25 bp, Homo sapiens
ttatgccagtcgtcataaagaaaac

SEQ ID NO: 873 - DNA, 25 bp, Homo sapiens
tatgccagtcgtcataaagaaaacc

SEQ ID NO: 874 - DNA, 25 bp, Homo sapiens
atgccagtcgtcataaagaaaacca

SEQ ID NO: 875 - DNA, 25 bp, Homo sapiens
tgccagtcgtcataaagaaaaccag

SEQ ID NO: 876 - DNA, 25 bp, Homo sapiens
gccagtcgtcataaagaaaaccagt

SEQ ID NO: 877 - DNA, 25 bp, Homo sapiens
ccagtcgtcataaagaaaaccagtg

SEQ ID NO: 878 - DNA, 25 bp, Homo sapiens
cagtcgtcataaagaaaaccagtgc

SEQ ID NO: 879 - DNA, 25 bp, Homo sapiens
agtcgtcataaagaaaaccagtgcc

SEQ ID NO: 880 - DNA, 25 bp, Homo sapiens
gtcgtcataaagaaaaccagtgcca

SEQ ID NO: 881 - DNA, 25 bp, Homo sapiens
tcgtcataaagaaaaccagtgccac

SEQ ID NO: 882 - DNA, 25 bp, Homo sapiens
cgtcataaagaaaaccagtgccact

SEQ ID NO: 883 - DNA, 25 bp, Homo sapiens
gtcataaagaaaaccagtgccactt

SEQ ID NO: 884 - DNA, 25 bp, Homo sapiens
tcataaagaaaaccagtgccacttc

SEQ ID NO: 885 - DNA, 25 bp, Homo sapiens
cataaagaaaaccagtgccacttca

SEQ ID NO: 886 - DNA, 25 bp, Homo sapiens
ataaagaaaaccagtgccacttcag

SEQ ID NO: 887 - DNA, 25 bp, Homo sapiens
taaagaaaaccagtgccacttcagt

SEQ ID NO: 888 - DNA, 25 bp, Homo sapiens
aaagaaaaccagtgccacttcagtc

SEQ ID NO: 889 - DNA, 25 bp, Homo sapiens
aagaaaaccagtgccacttcagtca

SEQ ID NO: 890 - DNA, 25 bp, Homo sapiens
agaaaaccagtgccacttcagtcaa

SEQ ID NO: 891 - DNA, 25 bp, Homo sapiens
gaaaaccagtgccacttcagtcaac

SEQ ID NO: 892 - DNA, 25 bp, Homo sapiens
aaaaccagtgccacttcagtcaacc

SEQ ID NO: 893 - DNA, 25 bp, Homo sapiens
aaaccagtgccacttcagtcaacct

SEQ ID NO: 894 - DNA, 25 bp, Homo sapiens
aaccagtgccacttcagtcaacctc

SEQ ID NO: 895 - DNA, 25 bp, Homo sapiens
accagtgccacttcagtcaacctct

SEQ ID NO: 896 - DNA, 25 bp, Homo sapiens
ccagtgccacttcagtcaacctctc

SEQ ID NO: 897 - DNA, 25 bp, Homo sapiens
cagtgccacttcagtcaacctctcc

SEQ ID NO: 898 - DNA, 25 bp, Homo sapiens
agtgccacttcagtcaacctctcct

SEQ ID NO: 899 - DNA, 25 bp, Homo sapiens
gtgccacttcagtcaacctctcctt

SEQ ID NO: 900 - DNA, 25 bp, Homo sapiens
tgccacttcagtcaacctctccttg

SEQ ID NO: 901 - DNA, 25 bp, Homo sapiens
gccacttcagtcaacctctccttgc

SEQ ID NO: 902 - DNA, 25 bp, Homo sapiens
ccacttcagtcaacctctccttgct

SEQ ID NO: 903 - DNA, 25 bp, Homo sapiens
cacttcagtcaacctctccttgctc

SEQ ID NO: 904 - DNA, 25 bp, Homo sapiens
acttcagtcaacctctccttgctca

SEQ ID NO: 905 - DNA, 17 bp, Homo sapiens
tgtgaaaggaacaagct

SEQ ID NO: 906 - DNA, 17 bp, Homo sapiens
gtgaaaggaacaagctc

SEQ ID NO: 907 - DNA, 17 bp, Homo sapiens
tgaaaggaacaagctca

SEQ ID NO: 908 - DNA, 17 bp, Homo sapiens
gaaaggaacaagctcaa

SEQ ID NO: 909 - DNA, 17 bp, Homo sapiens
aaaggaacaagctcaac

SEQ ID NO: 910 - DNA, 17 bp, Homo sapiens
aaggaacaagctcaaca

SEQ ID NO: 911 - DNA, 17 bp, Homo sapiens
aggaacaagctcaacat

SEQ ID NO: 912 - DNA, 17 bp, Homo sapiens
ggaacaagctcaacatc

SEQ ID NO: 913 - DNA, 17 bp, Homo sapiens
gaacaagctcaacatca

SEQ ID NO: 914 - DNA, 17 bp, Homo sapiens
aacaagctcaacatcac

SEQ ID NO: 915 - DNA, 17 bp, Homo sapiens
acaagctcaacatcact

SEQ ID NO: 916 - DNA, 17 bp, Homo sapiens
caagctcaacatcactg

SEQ ID NO: 917 - DNA, 17 bp, Homo sapiens
aagctcaacatcactgg

SEQ ID NO: 918 - DNA, 17 bp, Homo sapiens
agctcaacatcactgga

SEQ ID NO: 919 - DNA, 17 bp, Homo sapiens
gctcaacatcactggag

SEQ ID NO: 920 - DNA, 17 bp, Homo sapiens
ctcaacatcactggaga

SEQ ID NO: 921 - DNA, 17 bp, Homo sapiens
tcaacatcactggagac

SEQ ID NO: 922 - DNA, 17 bp, Homo sapiens
caacatcactggagaca

SEQ ID NO: 923 - DNA, 17 bp, Homo sapiens
aacatcactggagacac

SEQ ID NO: 924 - DNA, 17 bp, Homo sapiens
acatcactggagacaca

SEQ ID NO: 925 - DNA, 17 bp, Homo sapiens
catcactggagacacag

SEQ ID NO: 926 - DNA, 17 bp, Homo sapiens
atcactggagacacagg

SEQ ID NO: 927 - DNA, 17 bp, Homo sapiens
tcactggagacacaggc

SEQ ID NO: 928 - DNA, 17 bp, Homo sapiens
cactggagacacaggct

SEQ ID NO: 929 - DNA, 17 bp, Homo sapiens
actggagacacaggctc

SEQ ID NO: 930 - DNA, 17 bp, Homo sapiens
ctggagacacaggctct

SEQ ID NO: 931 - DNA, 17 bp, Homo sapiens
tggagacacaggctcta

SEQ ID NO: 932 - DNA, 17 bp, Homo sapiens
ggagacacaggctctaa

SEQ ID NO: 933 - DNA, 17 bp, Homo sapiens
gagacacaggctctaat

SEQ ID NO: 934 - DNA, 17 bp, Homo sapiens
agacacaggctctaatc

SEQ ID NO: 935 - DNA, 17 bp, Homo sapiens
gacacaggctctaatcc

SEQ ID NO: 936 - DNA, 17 bp, Homo sapiens
acacaggctctaatcca

SEQ ID NO: 937 - DNA, 17 bp, Homo sapiens
cacaggctctaatccac

SEQ ID NO: 938 - DNA, 17 bp, Homo sapiens
acaggctctaatccacc

SEQ ID NO: 939 - DNA, 17 bp, Homo sapiens
caggctctaatccacct

SEQ ID NO: 940 - DNA, 17 bp, Homo sapiens
aggctctaatccacctg

SEQ ID NO: 941 - DNA, 17 bp, Homo sapiens
ggctctaatccacctga

SEQ ID NO: 942 - DNA, 17 bp, Homo sapiens
gctctaatccacctgac

SEQ ID NO: 943 - DNA, 17 bp, Homo sapiens
ctctaatccacctgact

SEQ ID NO: 944 - DNA, 17 bp, Homo sapiens
tctaatccacctgactt

SEQ ID NO: 945 - DNA, 17 bp, Homo sapiens
ctaatccacctgacttg

SEQ ID NO: 946 - DNA, 17 bp, Homo sapiens
taatccacctgacttgg

SEQ ID NO: 947 - DNA, 17 bp, Homo sapiens
aatccacctgacttgga

SEQ ID NO: 948 - DNA, 17 bp, Homo sapiens
atccacctgacttggaa

SEQ ID NO: 949 - DNA, 17 bp, Homo sapiens
tccacctgacttggaag

SEQ ID NO: 950 - DNA, 17 bp, Homo sapiens
ccacctgacttggaagg

SEQ ID NO: 951 - DNA, 17 bp, Homo sapiens
cacctgacttggaagga

SEQ ID NO: 952 - DNA, 17 bp, Homo sapiens
acctgacttggaaggat

SEQ ID NO: 953 - DNA, 17 bp, Homo sapiens
cctgacttggaaggatg

SEQ ID NO: 954 - DNA, 17 bp, Homo sapiens
ctgacttggaaggatgc

SEQ ID NO: 955 - DNA, 17 bp, Homo sapiens
tgacttggaaggatgcc

SEQ ID NO: 956 - DNA, 17 bp, Homo sapiens
gacttggaaggatgcct

SEQ ID NO: 957 - DNA, 17 bp, Homo sapiens
acttggaaggatgccta

SEQ ID NO: 958 - DNA, 17 bp, Homo sapiens
cttggaaggatgcctag

SEQ ID NO: 959 - DNA, 17 bp, Homo sapiens
ttggaaggatgcctagc

SEQ ID NO: 960 - DNA, 17 bp, Homo sapiens
tggaaggatgcctagct

SEQ ID NO: 961 - DNA, 17 bp, Homo sapiens
ggaaggatgcctagcta

SEQ ID NO: 962 - DNA, 17 bp, Homo sapiens
gaaggatgcctagctaa

SEQ ID NO: 963 - DNA, 17 bp, Homo sapiens
aaggatgcctagctaac

SEQ ID NO: 964 - DNA, 17 bp, Homo sapiens
aggatgcctagctaacg

SEQ ID NO: 965 - DNA, 17 bp, Homo sapiens
ggatgcctagctaacgt

SEQ ID NO: 966 - DNA, 17 bp, Homo sapiens
gatgcctagctaacgtg

SEQ ID NO: 967 - DNA, 17 bp, Homo sapiens
atgcctagctaacgtgt

SEQ ID NO: 968 - DNA, 17 bp, Homo sapiens
tgcctagctaacgtgta

SEQ ID NO: 969 - DNA, 17 bp, Homo sapiens
gcctagctaacgtgtat

SEQ ID NO: 970 - DNA, 17 bp, Homo sapiens
cctagctaacgtgtatc

SEQ ID NO: 971 - DNA, 17 bp, Homo sapiens
ctagctaacgtgtatcc

SEQ ID NO: 972 - DNA, 17 bp, Homo sapiens
tagctaacgtgtatcct

SEQ ID NO: 973 - DNA, 17 bp, Homo sapiens
agctaacgtgtatcctg

SEQ ID NO: 974 - DNA, 17 bp, Homo sapiens
gctaacgtgtatcctga

SEQ ID NO: 975 - DNA, 17 bp, Homo sapiens
ctaacgtgtatcctgat

SEQ ID NO: 976 - DNA, 17 bp, Homo sapiens
taacgtgtatcctgatc

SEQ ID NO: 977 - DNA, 17 bp, Homo sapiens
aacgtgtatcctgatca

SEQ ID NO: 978 - DNA, 17 bp, Homo sapiens
acgtgtatcctgatcac

SEQ ID NO: 979 - DNA, 17 bp, Homo sapiens
cgtgtatcctgatcaca

SEQ ID NO: 980 - DNA, 17 bp, Homo sapiens
gtgtatcctgatcacaa

SEQ ID NO: 981 - DNA, 17 bp, Homo sapiens
tgtatcctgatcacaac

SEQ ID NO: 982 - DNA, 17 bp, Homo sapiens
gtatcctgatcacaact

SEQ ID NO: 983 - DNA, 17 bp, Homo sapiens
tatcctgatcacaactc

SEQ ID NO: 984 - DNA, 17 bp, Homo sapiens
atcctgatcacaactcc

SEQ ID NO: 985 - DNA, 17 bp, Homo sapiens
tcctgatcacaactccc

SEQ ID NO: 986 - DNA, 17 bp, Homo sapiens
cctgatcacaactcccc

SEQ ID NO: 987 - DNA, 17 bp, Homo sapiens
ctgatcacaactcccct

SEQ ID NO: 988 - DNA, 17 bp, Homo sapiens
tgatcacaactcccctg

SEQ ID NO: 989 - DNA, 17 bp, Homo sapiens
gatcacaactcccctgc

SEQ ID NO: 990 - DNA, 17 bp, Homo sapiens
atcacaactcccctgca

SEQ ID NO: 991 - DNA, 17 bp, Homo sapiens
tcacaactcccctgcat

SEQ ID NO: 992 - DNA, 17 bp, Homo sapiens
cacaactcccctgcatt

SEQ ID NO: 993 - DNA, 17 bp, Homo sapiens
acaactcccctgcattc

SEQ ID NO: 994 - DNA, 17 bp, Homo sapiens
caactcccctgcattca

SEQ ID NO: 995 - DNA, 17 bp, Homo sapiens
aactcccctgcattcat

SEQ ID NO: 996 - DNA, 17 bp, Homo sapiens
actcccctgcattcatt

SEQ ID NO: 997 - DNA, 17 bp, Homo sapiens
ctcccctgcattcatta

SEQ ID NO: 998 - DNA, 17 bp, Homo sapiens
tcccctgcattcattac

SEQ ID NO: 999 - DNA, 17 bp, Homo sapiens
cccctgcattcattact

SEQ ID NO: 1000 - DNA, 17 bp, Homo sapiens
ccctgcattcattactg

SEQ ID NO: 1001 - DNA, 17 bp, Homo sapiens
cctgcattcattactgt

SEQ ID NO: 1002 - DNA, 17 bp, Homo sapiens
ctgcattcattactgtg

SEQ ID NO: 1003 - DNA, 17 bp, Homo sapiens
tgcattcattactgtga

SEQ ID NO: 1004 - DNA, 17 bp, Homo sapiens
gcattcattactgtgaa

SEQ ID NO: 1005 - DNA, 17 bp, Homo sapiens
cattcattactgtgaaa

SEQ ID NO: 1006 - DNA, 17 bp, Homo sapiens
attcattactgtgaaaa

SEQ ID NO: 1007 - DNA, 17 bp, Homo sapiens
ttcattactgtgaaaat

SEQ ID NO: 1008 - DNA, 17 bp, Homo sapiens
tcattactgtgaaaatt

SEQ ID NO: 1009 - DNA, 17 bp, Homo sapiens
cattactgtgaaaattt

SEQ ID NO: 1010 - DNA, 17 bp, Homo sapiens
attactgtgaaaatttt

SEQ ID NO: 1011 - DNA, 17 bp, Homo sapiens
ttactgtgaaaattttt

SEQ ID NO: 1012 - DNA, 17 bp, Homo sapiens
tactgtgaaaatttttg

SEQ ID NO: 1013 - DNA, 17 bp, Homo sapiens
actgtgaaaatttttga

SEQ ID NO: 1014 - DNA, 17 bp, Homo sapiens
ctgtgaaaatttttgaa

SEQ ID NO: 1015 - DNA, 17 bp, Homo sapiens
tgtgaaaatttttgaat

SEQ ID NO: 1016 - DNA, 17 bp, Homo sapiens
gtgaaaatttttgaatc

SEQ ID NO: 1017 - DNA, 17 bp, Homo sapiens
tgaaaatttttgaatca

SEQ ID NO: 1018 - DNA, 17 bp, Homo sapiens
gaaaatttttgaatcaa

SEQ ID NO: 1019 - DNA, 17 bp, Homo sapiens
aaaatttttgaatcaaa

SEQ ID NO: 1020 - DNA, 17 bp, Homo sapiens
aaatttttgaatcaaaa

SEQ ID NO: 1021 - DNA, 17 bp, Homo sapiens
aatttttgaatcaaaag

SEQ ID NO: 1022 - DNA, 17 bp, Homo sapiens
atttttgaatcaaaagc

SEQ ID NO: 1023 - DNA, 17 bp, Homo sapiens
tttttgaatcaaaagca

SEQ ID NO: 1024 - DNA, 17 bp, Homo sapiens
ttttgaatcaaaagcaa

SEQ ID NO: 1025 - DNA, 17 bp, Homo sapiens
tttgaatcaaaagcaag

SEQ ID NO: 1026 - DNA, 17 bp, Homo sapiens
ttgaatcaaaagcaagt

SEQ ID NO: 1027 - DNA, 17 bp, Homo sapiens
tgaatcaaaagcaagta

SEQ ID NO: 1028 - DNA, 17 bp, Homo sapiens
gaatcaaaagcaagtat

SEQ ID NO: 1029 - DNA, 17 bp, Homo sapiens
aatcaaaagcaagtatt

SEQ ID NO: 1030 - DNA, 17 bp, Homo sapiens
atcaaaagcaagtatta

SEQ ID NO: 1031 - DNA, 17 bp, Homo sapiens
tcaaaagcaagtattat

SEQ ID NO: 1032 - DNA, 17 bp, Homo sapiens
caaaagcaagtattatt

SEQ ID NO: 1033 - DNA, 17 bp, Homo sapiens
aaaagcaagtattattg

SEQ ID NO: 1034 - DNA, 17 bp, Homo sapiens
aaagcaagtattattgt

SEQ ID NO: 1035 - DNA, 17 bp, Homo sapiens
aagcaagtattattgtt

SEQ ID NO: 1036 - DNA, 17 bp, Homo sapiens
agcaagtattattgtta

SEQ ID NO: 1037 - DNA, 17 bp, Homo sapiens
gcaagtattattgttat

SEQ ID NO: 1038 - DNA, 17 bp, Homo sapiens
caagtattattgttatt

SEQ ID NO: 1039 - DNA, 17 bp, Homo sapiens
aagtattattgttatta

SEQ ID NO: 1040 - DNA, 17 bp, Homo sapiens
agtattattgttattat

SEQ ID NO: 1041 - DNA, 17 bp, Homo sapiens
gtattattgttattatg

SEQ ID NO: 1042 - DNA, 17 bp, Homo sapiens
tattattgttattatga

SEQ ID NO: 1043 - DNA, 17 bp, Homo sapiens
attattgttattatgat

SEQ ID NO: 1044 - DNA, 17 bp, Homo sapiens
ttattgttattatgatt

SEQ ID NO: 1045 - DNA, 17 bp, Homo sapiens
tattgttattatgattt

SEQ ID NO: 1046 - DNA, 17 bp, Homo sapiens
attgttattatgatttg

SEQ ID NO: 1047 - DNA, 17 bp, Homo sapiens
ttgttattatgatttgt

SEQ ID NO: 1048 - DNA, 17 bp, Homo sapiens
tgttattatgatttgta

SEQ ID NO: 1049 - DNA, 17 bp, Homo sapiens
gttattatgatttgtat

SEQ ID NO: 1050 - DNA, 17 bp, Homo sapiens
ttattatgatttgtatt

SEQ ID NO: 1051 - DNA, 17 bp, Homo sapiens
tattatgatttgtatta

SEQ ID NO: 1052 - DNA, 17 bp, Homo sapiens
attatgatttgtattat

SEQ ID NO: 1053 - DNA, 17 bp, Homo sapiens
ttatgatttgtattatt

SEQ ID NO: 1054 - DNA, 17 bp, Homo sapiens
tatgatttgtattatta

SEQ ID NO: 1055 - DNA, 17 bp, Homo sapiens
atgatttgtattattat

SEQ ID NO: 1056 - DNA, 17 bp, Homo sapiens
tgatttgtattattatt

SEQ ID NO: 1057 - DNA, 17 bp, Homo sapiens
gatttgtattattatta

SEQ ID NO: 1058 - DNA, 17 bp, Homo sapiens
atttgtattattattat

SEQ ID NO: 1059 - DNA, 17 bp, Homo sapiens
tttgtattattattatt

SEQ ID NO: 1060 - DNA, 17 bp, Homo sapiens
ttgtattattattattg

SEQ ID NO: 1061 - DNA, 17 bp, Homo sapiens
tgtattattattattgc

SEQ ID NO: 1062 - DNA, 17 bp, Homo sapiens
gtattattattattgct

SEQ ID NO: 1063 - DNA, 17 bp, Homo sapiens
tattattattattgctg

SEQ ID NO: 1064 - DNA, 17 bp, Homo sapiens
attattattattgctgt

SEQ ID NO: 1065 - DNA, 17 bp, Homo sapiens
ttattattattgctgtt

SEQ ID NO: 1066 - DNA, 17 bp, Homo sapiens
tattattattgctgtta

SEQ ID NO: 1067 - DNA, 17 bp, Homo sapiens
attattattgctgttac

SEQ ID NO: 1068 - DNA, 17 bp, Homo sapiens
ttattattgctgttact

SEQ ID NO: 1069 - DNA, 17 bp, Homo sapiens
tattattgctgttactg

SEQ ID NO: 1070 - DNA, 17 bp, Homo sapiens
attattgctgttactgt

SEQ ID NO: 1071 - DNA, 17 bp, Homo sapiens
ttattgctgttactgtt

SEQ ID NO: 1072 - DNA, 17 bp, Homo sapiens
tattgctgttactgtta

SEQ ID NO: 1073 - DNA, 17 bp, Homo sapiens
attgctgttactgttat

SEQ ID NO: 1074 - DNA, 17 bp, Homo sapiens
ttgctgttactgttatt

SEQ ID NO: 1075 - DNA, 17 bp, Homo sapiens
tgctgttactgttatta

SEQ ID NO: 1076 - DNA, 17 bp, Homo sapiens
gctgttactgttattac

SEQ ID NO: 1077 - DNA, 17 bp, Homo sapiens
ctgttactgttattaca

SEQ ID NO: 1078 - DNA, 17 bp, Homo sapiens
tgttactgttattacac

SEQ ID NO: 1079 - DNA, 17 bp, Homo sapiens
gttactgttattacacc

SEQ ID NO: 1080 - DNA, 17 bp, Homo sapiens
ttactgttattacacca

SEQ ID NO: 1081 - DNA, 17 bp, Homo sapiens
tactgttattacaccaa

SEQ ID NO: 1082 - DNA, 17 bp, Homo sapiens
actgttattacaccaac

SEQ ID NO: 1083 - DNA, 17 bp, Homo sapiens
ctgttattacaccaact

SEQ ID NO: 1084 - DNA, 17 bp, Homo sapiens
tgttattacaccaactt

SEQ ID NO: 1085 - DNA, 17 bp, Homo sapiens
gttattacaccaacttt

SEQ ID NO: 1086 - DNA, 17 bp, Homo sapiens
ttattacaccaactttc

SEQ ID NO: 1087 - DNA, 17 bp, Homo sapiens
tattacaccaactttca

SEQ ID NO: 1088 - DNA, 17 bp, Homo sapiens
attacaccaactttcag

SEQ ID NO: 1089 - DNA, 17 bp, Homo sapiens
ttacaccaactttcagg

SEQ ID NO: 1090 - DNA, 17 bp, Homo sapiens
tacaccaactttcagga

SEQ ID NO: 1091 - DNA, 17 bp, Homo sapiens
acaccaactttcaggag

SEQ ID NO: 1092 - DNA, 17 bp, Homo sapiens
caccaactttcaggagg

SEQ ID NO: 1093 - DNA, 17 bp, Homo sapiens
accaactttcaggagga

SEQ ID NO: 1094 - DNA, 17 bp, Homo sapiens
ccaactttcaggaggat

SEQ ID NO: 1095 - DNA, 17 bp, Homo sapiens
caactttcaggaggatg

SEQ ID NO: 1096 - DNA, 17 bp, Homo sapiens
aactttcaggaggatgt

SEQ ID NO: 1097 - DNA, 17 bp, Homo sapiens
actttcaggaggatgtt

SEQ ID NO: 1098 - DNA, 17 bp, Homo sapiens
ctttcaggaggatgttt

SEQ ID NO: 1099 - DNA, 17 bp, Homo sapiens
tttcaggaggatgtttt

SEQ ID NO: 1100 - DNA, 17 bp, Homo sapiens
ttcaggaggatgttttg

SEQ ID NO: 1101 - DNA, 17 bp, Homo sapiens
tcaggaggatgttttgt

SEQ ID NO: 1102 - DNA, 17 bp, Homo sapiens
caggaggatgttttgta

SEQ ID NO: 1103 - DNA, 17 bp, Homo sapiens
aggaggatgttttgtac

SEQ ID NO: 1104 - DNA, 17 bp, Homo sapiens
ggaggatgttttgtact

SEQ ID NO: 1105 - DNA, 17 bp, Homo sapiens
gaggatgttttgtactc

SEQ ID NO: 1106 - DNA, 17 bp, Homo sapiens
aggatgttttgtactcc

SEQ ID NO: 1107 - DNA, 17 bp, Homo sapiens
ggatgttttgtactcct

SEQ ID NO: 1108 - DNA, 17 bp, Homo sapiens
gatgttttgtactcctg

SEQ ID NO: 1109 - DNA, 17 bp, Homo sapiens
atgttttgtactcctga

SEQ ID NO: 1110 - DNA, 17 bp, Homo sapiens
tgttttgtactcctgat

SEQ ID NO: 1111 - DNA, 17 bp, Homo sapiens
gttttgtactcctgatg

SEQ ID NO: 1112 - DNA, 17 bp, Homo sapiens
ttttgtactcctgatgg

SEQ ID NO: 1113 - DNA, 17 bp, Homo sapiens
tttgtactcctgatgga

SEQ ID NO: 1114 - DNA, 17 bp, Homo sapiens
ttgtactcctgatggaa

SEQ ID NO: 1115 - DNA, 17 bp, Homo sapiens
tgtactcctgatggaaa

SEQ ID NO: 1116 - DNA, 17 bp, Homo sapiens
gtactcctgatggaaac

SEQ ID NO: 1117 - DNA, 17 bp, Homo sapiens
tactcctgatggaaact

SEQ ID NO: 1118 - DNA, 17 bp, Homo sapiens
actcctgatggaaacta

SEQ ID NO: 1119 - DNA, 17 bp, Homo sapiens
ctcctgatggaaactat

SEQ ID NO: 1120 - DNA, 17 bp, Homo sapiens
tcctgatggaaactatt

SEQ ID NO: 1121 - DNA, 17 bp, Homo sapiens
cctgatggaaactattg

SEQ ID NO: 1122 - DNA, 17 bp, Homo sapiens
ctgatggaaactattgc

SEQ ID NO: 1123 - DNA, 17 bp, Homo sapiens
tgatggaaactattgcc

SEQ ID NO: 1124 - DNA, 17 bp, Homo sapiens
gatggaaactattgcca

SEQ ID NO: 1125 - DNA, 17 bp, Homo sapiens
atggaaactattgccag

SEQ ID NO: 1126 - DNA, 17 bp, Homo sapiens
tggaaactattgccaga

SEQ ID NO: 1127 - DNA, 17 bp, Homo sapiens
ggaaactattgccagac

SEQ ID NO: 1128 - DNA, 17 bp, Homo sapiens
gaaactattgccagacc

SEQ ID NO: 1129 - DNA, 17 bp, Homo sapiens
aaactattgccagaccc

SEQ ID NO: 1130 - DNA, 17 bp, Homo sapiens
aactattgccagaccca

SEQ ID NO: 1131 - DNA, 17 bp, Homo sapiens
actattgccagacccac

SEQ ID NO: 1132 - DNA, 17 bp, Homo sapiens
ctattgccagacccaca

SEQ ID NO: 1133 - DNA, 17 bp, Homo sapiens
tattgccagacccacat

SEQ ID NO: 1134 - DNA, 17 bp, Homo sapiens
attgccagacccacatg

SEQ ID NO: 1135 - DNA, 17 bp, Homo sapiens
ttgccagacccacatgt

SEQ ID NO: 1136 - DNA, 17 bp, Homo sapiens
tgccagacccacatgta

SEQ ID NO: 1137 - DNA, 17 bp, Homo sapiens
gccagacccacatgtag

SEQ ID NO: 1138 - DNA, 17 bp, Homo sapiens
ccagacccacatgtagt

SEQ ID NO: 1139 - DNA, 17 bp, Homo sapiens
cagacccacatgtagtc

SEQ ID NO: 1140 - DNA, 17 bp, Homo sapiens
agacccacatgtagtca

SEQ ID NO: 1141 - DNA, 17 bp, Homo sapiens
gacccacatgtagtcaa

SEQ ID NO: 1142 - DNA, 17 bp, Homo sapiens
acccacatgtagtcaac

SEQ ID NO: 1143 - DNA, 17 bp, Homo sapiens
cccacatgtagtcaaca

SEQ ID NO: 1144 - DNA, 17 bp, Homo sapiens
ccacatgtagtcaacat

SEQ ID NO: 1145 - DNA, 17 bp, Homo sapiens
cacatgtagtcaacata

SEQ ID NO: 1146 - DNA, 17 bp, Homo sapiens
acatgtagtcaacataa

SEQ ID NO: 1147 - DNA, 17 bp, Homo sapiens
catgtagtcaacataaa

SEQ ID NO: 1148 - DNA, 17 bp, Homo sapiens
atgtagtcaacataaac

SEQ ID NO: 1149 - DNA, 17 bp, Homo sapiens
tgtagtcaacataaacc

SEQ ID NO: 1150 - DNA, 17 bp, Homo sapiens
gtagtcaacataaaccc

SEQ ID NO: 1151 - DNA, 17 bp, Homo sapiens
tagtcaacataaacccc

SEQ ID NO: 1152 - DNA, 17 bp, Homo sapiens
agtcaacataaacccca

SEQ ID NO: 1153 - DNA, 17 bp, Homo sapiens
gtcaacataaaccccac

SEQ ID NO: 1154 - DNA, 17 bp, Homo sapiens
tcaacataaaccccact

SEQ ID NO: 1155 - DNA, 17 bp, Homo sapiens
caacataaaccccactt

SEQ ID NO: 1156 - DNA, 17 bp, Homo sapiens
aacataaaccccacttt

SEQ ID NO: 1157 - DNA, 17 bp, Homo sapiens
acataaaccccactttt

SEQ ID NO: 1158 - DNA, 17 bp, Homo sapiens
cataaaccccacttttc

SEQ ID NO: 1159 - DNA, 17 bp, Homo sapiens
ataaaccccacttttct

SEQ ID NO: 1160 - DNA, 17 bp, Homo sapiens
taaaccccacttttcta

SEQ ID NO: 1161 - DNA, 17 bp, Homo sapiens
aaaccccacttttctat

SEQ ID NO: 1162 - DNA, 17 bp, Homo sapiens
aaccccacttttctatg

SEQ ID NO: 1163 - DNA, 17 bp, Homo sapiens
accccacttttctatgg

SEQ ID NO: 1164 - DNA, 17 bp, Homo sapiens
ccccacttttctatggc

SEQ ID NO: 1165 - DNA, 17 bp, Homo sapiens
cccacttttctatggca

SEQ ID NO: 1166 - DNA, 17 bp, Homo sapiens
ccacttttctatggcaa

SEQ ID NO: 1167 - DNA, 17 bp, Homo sapiens
cacttttctatggcaat

SEQ ID NO: 1168 - DNA, 17 bp, Homo sapiens
acttttctatggcaatt

SEQ ID NO: 1169 - DNA, 17 bp, Homo sapiens
cttttctatggcaattc

SEQ ID NO: 1170 - DNA, 17 bp, Homo sapiens
ttttctatggcaattca

SEQ ID NO: 1171 - DNA, 17 bp, Homo sapiens
tttctatggcaattcac

SEQ ID NO: 1172 - DNA, 17 bp, Homo sapiens
ttctatggcaattcact

SEQ ID NO: 1173 - DNA, 17 bp, Homo sapiens
tctatggcaattcactt

SEQ ID NO: 1174 - DNA, 17 bp, Homo sapiens
ctatggcaattcacttt

SEQ ID NO: 1175 - DNA, 17 bp, Homo sapiens
tatggcaattcactttt

SEQ ID NO: 1176 - DNA, 17 bp, Homo sapiens
atggcaattcacttttt

SEQ ID NO: 1177 - DNA, 17 bp, Homo sapiens
tggcaattcacttttta

SEQ ID NO: 1178 - DNA, 17 bp, Homo sapiens
ggcaattcactttttaa

SEQ ID NO: 1179 - DNA, 17 bp, Homo sapiens
gcaattcactttttaag

SEQ ID NO: 1180 - DNA, 17 bp, Homo sapiens
caattcactttttaaga

SEQ ID NO: 1181 - DNA, 17 bp, Homo sapiens
aattcactttttaagag

SEQ ID NO: 1182 - DNA, 17 bp, Homo sapiens
attcactttttaagagt

SEQ ID NO: 1183 - DNA, 17 bp, Homo sapiens
ttcactttttaagagtc

SEQ ID NO: 1184 - DNA, 17 bp, Homo sapiens
tcactttttaagagtca

SEQ ID NO: 1185 - DNA, 17 bp, Homo sapiens
cactttttaagagtcat

SEQ ID NO: 1186 - DNA, 17 bp, Homo sapiens
actttttaagagtcata

SEQ ID NO: 1187 - DNA, 17 bp, Homo sapiens
ctttttaagagtcatac

SEQ ID NO: 1188 - DNA, 17 bp, Homo sapiens
tttttaagagtcatact

SEQ ID NO: 1189 - DNA, 17 bp, Homo sapiens
ttttaagagtcatactt

SEQ ID NO: 1190 - DNA, 17 bp, Homo sapiens
tttaagagtcatacttt

SEQ ID NO: 1191 - DNA, 17 bp, Homo sapiens
ttaagagtcatacttta

SEQ ID NO: 1192 - DNA, 17 bp, Homo sapiens
taagagtcatactttat

SEQ ID NO: 1193 - DNA, 17 bp, Homo sapiens
aagagtcatactttatt

SEQ ID NO: 1194 - DNA, 17 bp, Homo sapiens
agagtcatactttattt

SEQ ID NO: 1195 - DNA, 17 bp, Homo sapiens
gagtcatactttatttt

SEQ ID NO: 1196 - DNA, 17 bp, Homo sapiens
agtcatactttattttt

SEQ ID NO: 1197 - DNA, 17 bp, Homo sapiens
gtcatactttatttttt

SEQ ID NO: 1198 - DNA, 17 bp, Homo sapiens
tcatactttattttttt

SEQ ID NO: 1199 - DNA, 17 bp, Homo sapiens
catactttatttttttg

SEQ ID NO: 1200 - DNA, 17 bp, Homo sapiens
atactttatttttttgc

SEQ ID NO: 1201 - DNA, 17 bp, Homo sapiens
tactttatttttttgca

SEQ ID NO: 1202 - DNA, 17 bp, Homo sapiens
actttatttttttgcac

SEQ ID NO: 1203 - DNA, 17 bp, Homo sapiens
ctttatttttttgcaca

SEQ ID NO: 1204 - DNA, 17 bp, Homo sapiens
tttatttttttgcacag

SEQ ID NO: 1205 - DNA, 17 bp, Homo sapiens
ttatttttttgcacaga

SEQ ID NO: 1206 - DNA, 17 bp, Homo sapiens
tatttttttgcacagac

SEQ ID NO: 1207 - DNA, 17 bp, Homo sapiens
atttttttgcacagact

SEQ ID NO: 1208 - DNA, 17 bp, Homo sapiens
tttttttgcacagacta

SEQ ID NO: 1209 - DNA, 17 bp, Homo sapiens
ttttttgcacagactat

SEQ ID NO: 1210 - DNA, 17 bp, Homo sapiens
tttttgcacagactata

SEQ ID NO: 1211 - DNA, 17 bp, Homo sapiens
ttttgcacagactatat

SEQ ID NO: 1212 - DNA, 17 bp, Homo sapiens
tttgcacagactatatg

SEQ ID NO: 1213 - DNA, 17 bp, Homo sapiens
ttgcacagactatatga

SEQ ID NO: 1214 - DNA, 17 bp, Homo sapiens
tgcacagactatatgag

SEQ ID NO: 1215 - DNA, 17 bp, Homo sapiens
gcacagactatatgagt

SEQ ID NO: 1216 - DNA, 17 bp, Homo sapiens
cacagactatatgagtg

SEQ ID NO: 1217 - DNA, 17 bp, Homo sapiens
acagactatatgagtga

SEQ ID NO: 1218 - DNA, 17 bp, Homo sapiens
cagactatatgagtgat

SEQ ID NO: 1219 - DNA, 17 bp, Homo sapiens
agactatatgagtgatg

SEQ ID NO: 1220 - DNA, 17 bp, Homo sapiens
gactatatgagtgatgc

SEQ ID NO: 1221 - DNA, 17 bp, Homo sapiens
actatatgagtgatgca

SEQ ID NO: 1222 - DNA, 17 bp, Homo sapiens
ctatatgagtgatgcat

SEQ ID NO: 1223 - DNA, 17 bp, Homo sapiens
tatatgagtgatgcatg

SEQ ID NO: 1224 - DNA, 17 bp, Homo sapiens
atatgagtgatgcatgc

SEQ ID NO: 1225 - DNA, 17 bp, Homo sapiens
tatgagtgatgcatgcc

SEQ ID NO: 1226 - DNA, 17 bp, Homo sapiens
atgagtgatgcatgcca

SEQ ID NO: 1227 - DNA, 17 bp, Homo sapiens
tgagtgatgcatgccac

SEQ ID NO: 1228 - DNA, 17 bp, Homo sapiens
gagtgatgcatgccaca

SEQ ID NO: 1229 - DNA, 17 bp, Homo sapiens
agtgatgcatgccacag

SEQ ID NO: 1230 - DNA, 17 bp, Homo sapiens
gtgatgcatgccacagg

SEQ ID NO: 1231 - DNA, 17 bp, Homo sapiens
tgatgcatgccacagga

SEQ ID NO: 1232 - DNA, 17 bp, Homo sapiens
gatgcatgccacaggag

SEQ ID NO: 1233 - DNA, 17 bp, Homo sapiens
atgcatgccacaggagc

SEQ ID NO: 1234 - DNA, 17 bp, Homo sapiens
tgcatgccacaggagct

SEQ ID NO: 1235 - DNA, 17 bp, Homo sapiens
gcatgccacaggagctc

SEQ ID NO: 1236 - DNA, 17 bp, Homo sapiens
catgccacaggagctcc

SEQ ID NO: 1237 - DNA, 17 bp, Homo sapiens
atgccacaggagctcca

SEQ ID NO: 1238 - DNA, 17 bp, Homo sapiens
tgccacaggagctccac

SEQ ID NO: 1239 - DNA, 17 bp, Homo sapiens
gccacaggagctccacc

SEQ ID NO: 1240 - DNA, 17 bp, Homo sapiens
ccacaggagctccaccc

SEQ ID NO: 1241 - DNA, 17 bp, Homo sapiens
cacaggagctccacccc

SEQ ID NO: 1242 - DNA, 17 bp, Homo sapiens
acaggagctccacccct

SEQ ID NO: 1243 - DNA, 17 bp, Homo sapiens
caggagctccacccctg

SEQ ID NO: 1244 - DNA, 17 bp, Homo sapiens
aggagctccacccctga

SEQ ID NO: 1245 - DNA, 17 bp, Homo sapiens
ggagctccacccctgag

SEQ ID NO: 1246 - DNA, 17 bp, Homo sapiens
gagctccacccctgaga

SEQ ID NO: 1247 - DNA, 17 bp, Homo sapiens
agctccacccctgagaa

SEQ ID NO: 1248 - DNA, 17 bp, Homo sapiens
gctccacccctgagaag

SEQ ID NO: 1249 - DNA, 17 bp, Homo sapiens
ctccacccctgagaagt

SEQ ID NO: 1250 - DNA, 17 bp, Homo sapiens
tccacccctgagaagtt

SEQ ID NO: 1251 - DNA, 17 bp, Homo sapiens
ccacccctgagaagttt

SEQ ID NO: 1252 - DNA, 17 bp, Homo sapiens
cacccctgagaagtttc

SEQ ID NO: 1253 - DNA, 17 bp, Homo sapiens
acccctgagaagtttcc

SEQ ID NO: 1254 - DNA, 17 bp, Homo sapiens
cccctgagaagtttcct

SEQ ID NO: 1255 - DNA, 17 bp, Homo sapiens
ccctgagaagtttcctt

SEQ ID NO: 1256 - DNA, 17 bp, Homo sapiens
cctgagaagtttccttt

SEQ ID NO: 1257 - DNA, 17 bp, Homo sapiens
ctgagaagtttcctttt

SEQ ID NO: 1258 - DNA, 17 bp, Homo sapiens
tgagaagtttccttttc

SEQ ID NO: 1259 - DNA, 17 bp, Homo sapiens
gagaagtttccttttcc

SEQ ID NO: 1260 - DNA, 17 bp, Homo sapiens
agaagtttccttttcct

SEQ ID NO: 1261 - DNA, 17 bp, Homo sapiens
gaagtttccttttccta

SEQ ID NO: 1262 - DNA, 17 bp, Homo sapiens
aagtttccttttcctag

SEQ ID NO: 1263 - DNA, 17 bp, Homo sapiens
agtttccttttcctagg

SEQ ID NO: 1264 - DNA, 17 bp, Homo sapiens
gtttccttttcctaggg

SEQ ID NO: 1265 - DNA, 17 bp, Homo sapiens
tttccttttcctaggga

SEQ ID NO: 1266 - DNA, 17 bp, Homo sapiens
ttccttttcctagggac

SEQ ID NO: 1267 - DNA, 17 bp, Homo sapiens
tccttttcctagggacc

SEQ ID NO: 1268 - DNA, 17 bp, Homo sapiens
ccttttcctagggacct

SEQ ID NO: 1269 - DNA, 17 bp, Homo sapiens
cttttcctagggacctg

SEQ ID NO: 1270 - DNA, 17 bp, Homo sapiens
ttttcctagggacctgg

SEQ ID NO: 1271 - DNA, 17 bp, Homo sapiens
tttcctagggacctggt

SEQ ID NO: 1272 - DNA, 17 bp, Homo sapiens
ttcctagggacctggtg

SEQ ID NO: 1273 - DNA, 17 bp, Homo sapiens
tcctagggacctggtgg

SEQ ID NO: 1274 - DNA, 17 bp, Homo sapiens
cctagggacctggtggt

SEQ ID NO: 1275 - DNA, 17 bp, Homo sapiens
ctagggacctggtggtt

SEQ ID NO: 1276 - DNA, 17 bp, Homo sapiens
tagggacctggtggtta

SEQ ID NO: 1277 - DNA, 17 bp, Homo sapiens
agggacctggtggttaa

SEQ ID NO: 1278 - DNA, 17 bp, Homo sapiens
gggacctggtggttaat

SEQ ID NO: 1279 - DNA, 17 bp, Homo sapiens
ggacctggtggttaatg

SEQ ID NO: 1280 - DNA, 17 bp, Homo sapiens
gacctggtggttaatgg

SEQ ID NO: 1281 - DNA, 17 bp, Homo sapiens
acctggtggttaatggt

SEQ ID NO: 1282 - DNA, 17 bp, Homo sapiens
cctggtggttaatggtt

SEQ ID NO: 1283 - DNA, 17 bp, Homo sapiens
ctggtggttaatggttt

SEQ ID NO: 1284 - DNA, 17 bp, Homo sapiens
tggtggttaatggtttc

SEQ ID NO: 1285 - DNA, 17 bp, Homo sapiens
ggtggttaatggtttcc

SEQ ID NO: 1286 - DNA, 17 bp, Homo sapiens
gtggttaatggtttcct

SEQ ID NO: 1287 - DNA, 17 bp, Homo sapiens
tggttaatggtttcctc

SEQ ID NO: 1288 - DNA, 17 bp, Homo sapiens
ggttaatggtttcctct

SEQ ID NO: 1289 - DNA, 17 bp, Homo sapiens
gttaatggtttcctctg

SEQ ID NO: 1290 - DNA, 17 bp, Homo sapiens
ttaatggtttcctctgt

SEQ ID NO: 1291 - DNA, 17 bp, Homo sapiens
taatggtttcctctgtt

SEQ ID NO: 1292 - DNA, 17 bp, Homo sapiens
aatggtttcctctgtta

SEQ ID NO: 1293 - DNA, 17 bp, Homo sapiens
atggtttcctctgttac

SEQ ID NO: 1294 - DNA, 17 bp, Homo sapiens
tggtttcctctgttacc

SEQ ID NO: 1295 - DNA, 17 bp, Homo sapiens
ggtttcctctgttacct

SEQ ID NO: 1296 - DNA, 17 bp, Homo sapiens
gtttcctctgttacctg

SEQ ID NO: 1297 - DNA, 17 bp, Homo sapiens
tttcctctgttacctgt

SEQ ID NO: 1298 - DNA, 17 bp, Homo sapiens
ttcctctgttacctgtt

SEQ ID NO: 1299 - DNA, 17 bp, Homo sapiens
tcctctgttacctgttg

SEQ ID NO: 1300 - DNA, 17 bp, Homo sapiens
cctctgttacctgttgg

SEQ ID NO: 1301 - DNA, 17 bp, Homo sapiens
ctctgttacctgttgga

SEQ ID NO: 1302 - DNA, 17 bp, Homo sapiens
tctgttacctgttggat

SEQ ID NO: 1303 - DNA, 17 bp, Homo sapiens
ctgttacctgttggatt

SEQ ID NO: 1304 - DNA, 17 bp, Homo sapiens
tgttacctgttggattg

SEQ ID NO: 1305 - DNA, 17 bp, Homo sapiens
gttacctgttggattgc

SEQ ID NO: 1306 - DNA, 17 bp, Homo sapiens
ttacctgttggattgcc

SEQ ID NO: 1307 - DNA, 17 bp, Homo sapiens
tacctgttggattgcct

SEQ ID NO: 1308 - DNA, 17 bp, Homo sapiens
acctgttggattgcctg

SEQ ID NO: 1309 - DNA, 17 bp, Homo sapiens
cctgttggattgcctgc

SEQ ID NO: 1310 - DNA, 17 bp, Homo sapiens
ctgttggattgcctgct

SEQ ID NO: 1311 - DNA, 17 bp, Homo sapiens
tgttggattgcctgctc

SEQ ID NO: 1312 - DNA, 17 bp, Homo sapiens
gttggattgcctgctca

SEQ ID NO: 1313 - DNA, 17 bp, Homo sapiens
ttggattgcctgctcaa

SEQ ID NO: 1314 - DNA, 17 bp, Homo sapiens
tggattgcctgctcaat

SEQ ID NO: 1315 - DNA, 17 bp, Homo sapiens
ggattgcctgctcaata

SEQ ID NO: 1316 - DNA, 17 bp, Homo sapiens
gattgcctgctcaataa

SEQ ID NO: 1317 - DNA, 17 bp, Homo sapiens
attgcctgctcaataag

SEQ ID NO: 1318 - DNA, 17 bp, Homo sapiens
ttgcctgctcaataagt

SEQ ID NO: 1319 - DNA, 17 bp, Homo sapiens
tgcctgctcaataagta

SEQ ID NO: 1320 - DNA, 17 bp, Homo sapiens
gcctgctcaataagtat

SEQ ID NO: 1321 - DNA, 17 bp, Homo sapiens
cctgctcaataagtatt

SEQ ID NO: 1322 - DNA, 17 bp, Homo sapiens
ctgctcaataagtattt

SEQ ID NO: 1323 - DNA, 17 bp, Homo sapiens
tgctcaataagtatttt

SEQ ID NO: 1324 - DNA, 17 bp, Homo sapiens
gctcaataagtattttg

SEQ ID NO: 1325 - DNA, 17 bp, Homo sapiens
ctcaataagtattttgt

SEQ ID NO: 1326 - DNA, 17 bp, Homo sapiens
tcaataagtattttgtg

SEQ ID NO: 1327 - DNA, 17 bp, Homo sapiens
caataagtattttgtgc

SEQ ID NO: 1328 - DNA, 17 bp, Homo sapiens
aataagtattttgtgct

SEQ ID NO: 1329 - DNA, 17 bp, Homo sapiens
ataagtattttgtgctg

SEQ ID NO: 1330 - DNA, 17 bp, Homo sapiens
taagtattttgtgctgt

SEQ ID NO: 1331 - DNA, 17 bp, Homo sapiens
aagtattttgtgctgtg

SEQ ID NO: 1332 - DNA, 17 bp, Homo sapiens
agtattttgtgctgtga

SEQ ID NO: 1333 - DNA, 17 bp, Homo sapiens
gtattttgtgctgtgac

SEQ ID NO: 1334 - DNA, 17 bp, Homo sapiens
tattttgtgctgtgacc

SEQ ID NO: 1335 - DNA, 17 bp, Homo sapiens
attttgtgctgtgacct

SEQ ID NO: 1336 - DNA, 17 bp, Homo sapiens
ttttgtgctgtgacctt

SEQ ID NO: 1337 - DNA, 17 bp, Homo sapiens
tttgtgctgtgaccttt

SEQ ID NO: 1338 - DNA, 17 bp, Homo sapiens
ttgtgctgtgacctttg

SEQ ID NO: 1339 - DNA, 17 bp, Homo sapiens
tgtgctgtgacctttgt

SEQ ID NO: 1340 - DNA, 17 bp, Homo sapiens
gtgctgtgacctttgtg

SEQ ID NO: 1341 - DNA, 17 bp, Homo sapiens
tgctgtgacctttgtgg

SEQ ID NO: 1342 - DNA, 17 bp, Homo sapiens
gctgtgacctttgtggg

SEQ ID NO: 1343 - DNA, 17 bp, Homo sapiens
ctgtgacctttgtgggg

SEQ ID NO: 1344 - DNA, 17 bp, Homo sapiens
tgtgacctttgtgggga

SEQ ID NO: 1345 - DNA, 17 bp, Homo sapiens
gtgacctttgtggggag

SEQ ID NO: 1346 - DNA, 17 bp, Homo sapiens
tgacctttgtggggagg

SEQ ID NO: 1347 - DNA, 17 bp, Homo sapiens
gacctttgtggggaggg

SEQ ID NO: 1348 - DNA, 17 bp, Homo sapiens
acctttgtggggagggg

SEQ ID NO: 1349 - DNA, 17 bp, Homo sapiens
cctttgtggggaggggc

SEQ ID NO: 1350 - DNA, 17 bp, Homo sapiens
ctttgtggggaggggca

SEQ ID NO: 1351 - DNA, 17 bp, Homo sapiens
tttgtggggaggggcaa

SEQ ID NO: 1352 - DNA, 17 bp, Homo sapiens
ttgtggggaggggcaac

SEQ ID NO: 1353 - DNA, 17 bp, Homo sapiens
tgtggggaggggcaact

SEQ ID NO: 1354 - DNA, 17 bp, Homo sapiens
gtggggaggggcaactg

SEQ ID NO: 1355 - DNA, 17 bp, Homo sapiens
tggggaggggcaactga

SEQ ID NO: 1356 - DNA, 17 bp, Homo sapiens
ggggaggggcaactgac

SEQ ID NO: 1357 - DNA, 17 bp, Homo sapiens
gggaggggcaactgacc

SEQ ID NO: 1358 - DNA, 17 bp, Homo sapiens
ggaggggcaactgacca

SEQ ID NO: 1359 - DNA, 17 bp, Homo sapiens
gaggggcaactgaccat

SEQ ID NO: 1360 - DNA, 17 bp, Homo sapiens
aggggcaactgaccata

SEQ ID NO: 1361 - DNA, 17 bp, Homo sapiens
ggggcaactgaccatat

SEQ ID NO: 1362 - DNA, 17 bp, Homo sapiens
gggcaactgaccatata

SEQ ID NO: 1363 - DNA, 17 bp, Homo sapiens
ggcaactgaccatatac

SEQ ID NO: 1364 - DNA, 17 bp, Homo sapiens
gcaactgaccatatact

SEQ ID NO: 1365 - DNA, 17 bp, Homo sapiens
caactgaccatatactt

SEQ ID NO: 1366 - DNA, 17 bp, Homo sapiens
aactgaccatatacttc

SEQ ID NO: 1367 - DNA, 17 bp, Homo sapiens
actgaccatatacttct

SEQ ID NO: 1368 - DNA, 17 bp, Homo sapiens
ctgaccatatacttctc

SEQ ID NO: 1369 - DNA, 17 bp, Homo sapiens
tgaccatatacttctct

SEQ ID NO: 1370 - DNA, 17 bp, Homo sapiens
gaccatatacttctcta

SEQ ID NO: 1371 - DNA, 17 bp, Homo sapiens
accatatacttctctat

SEQ ID NO: 1372 - DNA, 17 bp, Homo sapiens
ccatatacttctctatt

SEQ ID NO: 1373 - DNA, 17 bp, Homo sapiens
catatacttctctattc

SEQ ID NO: 1374 - DNA, 17 bp, Homo sapiens
atatacttctctattct

SEQ ID NO: 1375 - DNA, 17 bp, Homo sapiens
tatacttctctattcta

SEQ ID NO: 1376 - DNA, 17 bp, Homo sapiens
atacttctctattctag

SEQ ID NO: 1377 - DNA, 17 bp, Homo sapiens
tacttctctattctagg

SEQ ID NO: 1378 - DNA, 17 bp, Homo sapiens
acttctctattctagga

SEQ ID NO: 1379 - DNA, 17 bp, Homo sapiens
cttctctattctaggaa

SEQ ID NO: 1380 - DNA, 17 bp, Homo sapiens
ttctctattctaggaat

SEQ ID NO: 1381 - DNA, 17 bp, Homo sapiens
tctctattctaggaata

SEQ ID NO: 1382 - DNA, 17 bp, Homo sapiens
ctctattctaggaatag

SEQ ID NO: 1383 - DNA, 17 bp, Homo sapiens
tctattctaggaataga

SEQ ID NO: 1384 - DNA, 17 bp, Homo sapiens
ctattctaggaatagat

SEQ ID NO: 1385 - DNA, 17 bp, Homo sapiens
tattctaggaatagata

SEQ ID NO: 1386 - DNA, 17 bp, Homo sapiens
attctaggaatagatat

SEQ ID NO: 1387 - DNA, 17 bp, Homo sapiens
ttctaggaatagatata

SEQ ID NO: 1388 - DNA, 17 bp, Homo sapiens
tctaggaatagatataa

SEQ ID NO: 1389 - DNA, 17 bp, Homo sapiens
ctaggaatagatataaa

SEQ ID NO: 1390 - DNA, 17 bp, Homo sapiens
taggaatagatataaaa

SEQ ID NO: 1391 - DNA, 17 bp, Homo sapiens
aggaatagatataaaag

SEQ ID NO: 1392 - DNA, 17 bp, Homo sapiens
ggaatagatataaaaga

SEQ ID NO: 1393 - DNA, 17 bp, Homo sapiens
gaatagatataaaagaa

SEQ ID NO: 1394 - DNA, 17 bp, Homo sapiens
aatagatataaaagaaa

SEQ ID NO: 1395 - DNA, 17 bp, Homo sapiens
atagatataaaagaaac

SEQ ID NO: 1396 - DNA, 17 bp, Homo sapiens
tagatataaaagaaaca

SEQ ID NO: 1397 - DNA, 17 bp, Homo sapiens
agatataaaagaaacat

SEQ ID NO: 1398 - DNA, 17 bp, Homo sapiens
gatataaaagaaacatt

SEQ ID NO: 1399 - DNA, 17 bp, Homo sapiens
atataaaagaaacattt

SEQ ID NO: 1400 - DNA, 17 bp, Homo sapiens
tataaaagaaacatttt

SEQ ID NO: 1401 - DNA, 17 bp, Homo sapiens
ataaaagaaacatttta

SEQ ID NO: 1402 - DNA, 17 bp, Homo sapiens
taaaagaaacattttag

SEQ ID NO: 1403 - DNA, 17 bp, Homo sapiens
aaaagaaacattttagc

SEQ ID NO: 1404 - DNA, 17 bp, Homo sapiens
aaagaaacattttagcc

SEQ ID NO: 1405 - DNA, 17 bp, Homo sapiens
aagaaacattttagcct

SEQ ID NO: 1406 - DNA, 17 bp, Homo sapiens
agaaacattttagcctt

SEQ ID NO: 1407 - DNA, 17 bp, Homo sapiens
gaaacattttagccttt

SEQ ID NO: 1408 - DNA, 17 bp, Homo sapiens
aaacattttagcctttt

SEQ ID NO: 1409 - DNA, 17 bp, Homo sapiens
aacattttagccttttt

SEQ ID NO: 1410 - DNA, 17 bp, Homo sapiens
acattttagccttttta

SEQ ID NO: 1411 - DNA, 17 bp, Homo sapiens
cattttagcctttttat

SEQ ID NO: 1412 - DNA, 17 bp, Homo sapiens
attttagcctttttata

SEQ ID NO: 1413 - DNA, 25 bp, Homo sapiens
tgtgaaaggaacaagctcaacatca

SEQ ID NO: 1414 - DNA, 25 bp, Homo sapiens
gtgaaaggaacaagctcaacatcac

SEQ ID NO: 1415 - DNA, 25 bp, Homo sapiens
tgaaaggaacaagctcaacatcact

SEQ ID NO: 1416 - DNA, 25 bp, Homo sapiens
gaaaggaacaagctcaacatcactg

SEQ ID NO: 1417 - DNA, 25 bp, Homo sapiens
aaaggaacaagctcaacatcactgg

SEQ ID NO: 1418 - DNA, 25 bp, Homo sapiens
aaggaacaagctcaacatcactgga

SEQ ID NO: 1419 - DNA, 25 bp, Homo sapiens
aggaacaagctcaacatcactggag

SEQ ID NO: 1420 - DNA, 25 bp, Homo sapiens
ggaacaagctcaacatcactggaga

SEQ ID NO: 1421 - DNA, 25 bp, Homo sapiens
gaacaagctcaacatcactggagac

SEQ ID NO: 1422 - DNA, 25 bp, Homo sapiens
aacaagctcaacatcactggagaca

SEQ ID NO: 1423 - DNA, 25 bp, Homo sapiens
acaagctcaacatcactggagacac

SEQ ID NO: 1424 - DNA, 25 bp, Homo sapiens
caagctcaacatcactggagacaca

SEQ ID NO: 1425 - DNA, 25 bp, Homo sapiens
aagctcaacatcactggagacacag

SEQ ID NO: 1426 - DNA, 25 bp, Homo sapiens
agctcaacatcactggagacacagg

SEQ ID NO: 1427 - DNA, 25 bp, Homo sapiens
gctcaacatcactggagacacaggc

SEQ ID NO: 1428 - DNA, 25 bp, Homo sapiens
ctcaacatcactggagacacaggct

SEQ ID NO: 1429 - DNA, 25 bp, Homo sapiens
tcaacatcactggagacacaggctc

SEQ ID NO: 1430 - DNA, 25 bp, Homo sapiens
caacatcactggagacacaggctct

SEQ ID NO: 1431 - DNA, 25 bp, Homo sapiens
aacatcactggagacacaggctcta

SEQ ID NO: 1432 - DNA, 25 bp, Homo sapiens
acatcactggagacacaggctctaa

SEQ ID NO: 1433 - DNA, 25 bp, Homo sapiens
catcactggagacacaggctctaat

SEQ ID NO: 1434 - DNA, 25 bp, Homo sapiens
atcactggagacacaggctctaatc

SEQ ID NO: 1435 - DNA, 25 bp, Homo sapiens
tcactggagacacaggctctaatcc

SEQ ID NO: 1436 - DNA, 25 bp, Homo sapiens
cactggagacacaggctctaatcca

SEQ ID NO: 1437 - DNA, 25 bp, Homo sapiens
actggagacacaggctctaatccac

SEQ ID NO: 1438 - DNA, 25 bp, Homo sapiens
ctggagacacaggctctaatccacc

SEQ ID NO: 1439 - DNA, 25 bp, Homo sapiens
tggagacacaggctctaatccacct

SEQ ID NO: 1440 - DNA, 25 bp, Homo sapiens
ggagacacaggctctaatccacctg

SEQ ID NO: 1441 - DNA, 25 bp, Homo sapiens
gagacacaggctctaatccacctga

SEQ ID NO: 1442 - DNA, 25 bp, Homo sapiens
agacacaggctctaatccacctgac

SEQ ID NO: 1443 - DNA, 25 bp, Homo sapiens
gacacaggctctaatccacctgact

SEQ ID NO: 1444 - DNA, 25 bp, Homo sapiens
acacaggctctaatccacctgactt

SEQ ID NO: 1445 - DNA, 25 bp, Homo sapiens
cacaggctctaatccacctgacttg

SEQ ID NO: 1446 - DNA, 25 bp, Homo sapiens
acaggctctaatccacctgacttgg

SEQ ID NO: 1447 - DNA, 25 bp, Homo sapiens
caggctctaatccacctgacttgga

SEQ ID NO: 1448 - DNA, 25 bp, Homo sapiens
aggctctaatccacctgacttggaa

SEQ ID NO: 1449 - DNA, 25 bp, Homo sapiens
ggctctaatccacctgacttggaag

SEQ ID NO: 1450 - DNA, 25 bp, Homo sapiens
gctctaatccacctgacttggaagg

SEQ ID NO: 1451 - DNA, 25 bp, Homo sapiens
ctctaatccacctgacttggaagga

SEQ ID NO: 1452 - DNA, 25 bp, Homo sapiens
tctaatccacctgacttggaaggat

SEQ ID NO: 1453 - DNA, 25 bp, Homo sapiens
ctaatccacctgacttggaaggatg

SEQ ID NO: 1454 - DNA, 25 bp, Homo sapiens
taatccacctgacttggaaggatgc

SEQ ID NO: 1455 - DNA, 25 bp, Homo sapiens
aatccacctgacttggaaggatgcc

SEQ ID NO: 1456 - DNA, 25 bp, Homo sapiens
atccacctgacttggaaggatgcct

SEQ ID NO: 1457 - DNA, 25 bp, Homo sapiens
tccacctgacttggaaggatgccta

SEQ ID NO: 1458 - DNA, 25 bp, Homo sapiens
ccacctgacttggaaggatgcctag

SEQ ID NO: 1459 - DNA, 25 bp, Homo sapiens
cacctgacttggaaggatgcctagc

SEQ ID NO: 1460 - DNA, 25 bp, Homo sapiens
acctgacttggaaggatgcctagct

SEQ ID NO: 1461 - DNA, 25 bp, Homo sapiens
cctgacttggaaggatgcctagcta

SEQ ID NO: 1462 - DNA, 25 bp, Homo sapiens
ctgacttggaaggatgcctagctaa

SEQ ID NO: 1463 - DNA, 25 bp, Homo sapiens
tgacttggaaggatgcctagctaac

SEQ ID NO: 1464 - DNA, 25 bp, Homo sapiens
gacttggaaggatgcctagctaacg

SEQ ID NO: 1465 - DNA, 25 bp, Homo sapiens
acttggaaggatgcctagctaacgt

SEQ ID NO: 1466 - DNA, 25 bp, Homo sapiens
cttggaaggatgcctagctaacgtg

SEQ ID NO: 1467 - DNA, 25 bp, Homo sapiens
ttggaaggatgcctagctaacgtgt

SEQ ID NO: 1468 - DNA, 25 bp, Homo sapiens
tggaaggatgcctagctaacgtgta

SEQ ID NO: 1469 - DNA, 25 bp, Homo sapiens
ggaaggatgcctagctaacgtgtat

SEQ ID NO: 1470 - DNA, 25 bp, Homo sapiens
gaaggatgcctagctaacgtgtatc

SEQ ID NO: 1471 - DNA, 25 bp, Homo sapiens
aaggatgcctagctaacgtgtatcc

SEQ ID NO: 1472 - DNA, 25 bp, Homo sapiens
aggatgcctagctaacgtgtatcct

SEQ ID NO: 1473 - DNA, 25 bp, Homo sapiens
ggatgcctagctaacgtgtatcctg

SEQ ID NO: 1474 - DNA, 25 bp, Homo sapiens
gatgcctagctaacgtgtatcctga

SEQ ID NO: 1475 - DNA, 25 bp, Homo sapiens
atgcctagctaacgtgtatcctgat

SEQ ID NO: 1476 - DNA, 25 bp, Homo sapiens
tgcctagctaacgtgtatcctgatc

SEQ ID NO: 1477 - DNA, 25 bp, Homo sapiens
gcctagctaacgtgtatcctgatca

SEQ ID NO: 1478 - DNA, 25 bp, Homo sapiens
cctagctaacgtgtatcctgatcac

SEQ ID NO: 1479 - DNA, 25 bp, Homo sapiens
ctagctaacgtgtatcctgatcaca

SEQ ID NO: 1480 - DNA, 25 bp, Homo sapiens
tagctaacgtgtatcctgatcacaa

SEQ ID NO: 1481 - DNA, 25 bp, Homo sapiens
agctaacgtgtatcctgatcacaac

SEQ ID NO: 1482 - DNA, 25 bp, Homo sapiens
gctaacgtgtatcctgatcacaact

SEQ ID NO: 1483 - DNA, 25 bp, Homo sapiens
ctaacgtgtatcctgatcacaactc

SEQ ID NO: 1484 - DNA, 25 bp, Homo sapiens
taacgtgtatcctgatcacaactcc

SEQ ID NO: 1485 - DNA, 25 bp, Homo sapiens
aacgtgtatcctgatcacaactccc

SEQ ID NO: 1486 - DNA, 25 bp, Homo sapiens
acgtgtatcctgatcacaactcccc

SEQ ID NO: 1487 - DNA, 25 bp, Homo sapiens
cgtgtatcctgatcacaactcccct

SEQ ID NO: 1488 - DNA, 25 bp, Homo sapiens
gtgtatcctgatcacaactcccctg

SEQ ID NO: 1489 - DNA, 25 bp, Homo sapiens
tgtatcctgatcacaactcccctgc

SEQ ID NO: 1490 - DNA, 25 bp, Homo sapiens
gtatcctgatcacaactcccctgca

SEQ ID NO: 1491 - DNA, 25 bp, Homo sapiens
tatcctgatcacaactcccctgcat

SEQ ID NO: 1492 - DNA, 25 bp, Homo sapiens
atcctgatcacaactcccctgcatt

SEQ ID NO: 1493 - DNA, 25 bp, Homo sapiens
tcctgatcacaactcccctgcattc

SEQ ID NO: 1494 - DNA, 25 bp, Homo sapiens
cctgatcacaactcccctgcattca

SEQ ID NO: 1495 - DNA, 25 bp, Homo sapiens
ctgatcacaactcccctgcattcat

SEQ ID NO: 1496 - DNA, 25 bp, Homo sapiens
tgatcacaactcccctgcattcatt

SEQ ID NO: 1497 - DNA, 25 bp, Homo sapiens
gatcacaactcccctgcattcatta

SEQ ID NO: 1498 - DNA, 25 bp, Homo sapiens
atcacaactcccctgcattcattac

SEQ ID NO: 1499 - DNA, 25 bp, Homo sapiens
tcacaactcccctgcattcattact

SEQ ID NO: 1500 - DNA, 25 bp, Homo sapiens
cacaactcccctgcattcattactg

SEQ ID NO: 1501 - DNA, 25 bp, Homo sapiens
acaactcccctgcattcattactgt

SEQ ID NO: 1502 - DNA, 25 bp, Homo sapiens
caactcccctgcattcattactgtg

SEQ ID NO: 1503 - DNA, 25 bp, Homo sapiens
aactcccctgcattcattactgtga

SEQ ID NO: 1504 - DNA, 25 bp, Homo sapiens
actcccctgcattcattactgtgaa

SEQ ID NO: 1505 - DNA, 25 bp, Homo sapiens
ctcccctgcattcattactgtgaaa

SEQ ID NO: 1506 - DNA, 25 bp, Homo sapiens
tcccctgcattcattactgtgaaaa

SEQ ID NO: 1507 - DNA, 25 bp, Homo sapiens
cccctgcattcattactgtgaaaat

SEQ ID NO: 1508 - DNA, 25 bp, Homo sapiens
ccctgcattcattactgtgaaaatt

SEQ ID NO: 1509 - DNA, 25 bp, Homo sapiens
cctgcattcattactgtgaaaattt

SEQ ID NO: 1510 - DNA, 25 bp, Homo sapiens
ctgcattcattactgtgaaaatttt

SEQ ID NO: 1511 - DNA, 25 bp, Homo sapiens
tgcattcattactgtgaaaattttt

SEQ ID NO: 1512 - DNA, 25 bp, Homo sapiens
gcattcattactgtgaaaatttttg

SEQ ID NO: 1513 - DNA, 25 bp, Homo sapiens
cattcattactgtgaaaatttttga

SEQ ID NO: 1514 - DNA, 25 bp, Homo sapiens
attcattactgtgaaaatttttgaa

SEQ ID NO: 1515 - DNA, 25 bp, Homo sapiens
ttcattactgtgaaaatttttgaat

SEQ ID NO: 1516 - DNA, 25 bp, Homo sapiens
tcattactgtgaaaatttttgaatc

SEQ ID NO: 1517 - DNA, 25 bp, Homo sapiens
cattactgtgaaaatttttgaatca

SEQ ID NO: 1518 - DNA, 25 bp, Homo sapiens
attactgtgaaaatttttgaatcaa

SEQ ID NO: 1519 - DNA, 25 bp, Homo sapiens
ttactgtgaaaatttttgaatcaaa

SEQ ID NO: 1520 - DNA, 25 bp, Homo sapiens
tactgtgaaaatttttgaatcaaaa

SEQ ID NO: 1521 - DNA, 25 bp, Homo sapiens
actgtgaaaatttttgaatcaaaag

SEQ ID NO: 1522 - DNA, 25 bp, Homo sapiens
ctgtgaaaatttttgaatcaaaagc

SEQ ID NO: 1523 - DNA, 25 bp, Homo sapiens
tgtgaaaatttttgaatcaaaagca

SEQ ID NO: 1524 - DNA, 25 bp, Homo sapiens
gtgaaaatttttgaatcaaaagcaa

SEQ ID NO: 1525 - DNA, 25 bp, Homo sapiens
tgaaaatttttgaatcaaaagcaag

SEQ ID NO: 1526 - DNA, 25 bp, Homo sapiens
gaaaatttttgaatcaaaagcaagt

SEQ ID NO: 1527 - DNA, 25 bp, Homo sapiens
aaaatttttgaatcaaaagcaagta

SEQ ID NO: 1528 - DNA, 25 bp, Homo sapiens
aaatttttgaatcaaaagcaagtat

SEQ ID NO: 1529 - DNA, 25 bp, Homo sapiens
aatttttgaatcaaaagcaagtatt

SEQ ID NO: 1530 - DNA, 25 bp, Homo sapiens
atttttgaatcaaaagcaagtatta

SEQ ID NO: 1531 - DNA, 25 bp, Homo sapiens
tttttgaatcaaaagcaagtattat

SEQ ID NO: 1532 - DNA, 25 bp, Homo sapiens
ttttgaatcaaaagcaagtattatt

SEQ ID NO: 1533 - DNA, 25 bp, Homo sapiens
tttgaatcaaaagcaagtattattg

SEQ ID NO: 1534 - DNA, 25 bp, Homo sapiens
ttgaatcaaaagcaagtattattgt

SEQ ID NO: 1535 - DNA, 25 bp, Homo sapiens
tgaatcaaaagcaagtattattgtt

SEQ ID NO: 1536 - DNA, 25 bp, Homo sapiens
gaatcaaaagcaagtattattgtta

SEQ ID NO: 1537 - DNA, 25 bp, Homo sapiens
aatcaaaagcaagtattattgttat

SEQ ID NO: 1538 - DNA, 25 bp, Homo sapiens
atcaaaagcaagtattattgttatt

SEQ ID NO: 1539 - DNA, 25 bp, Homo sapiens
tcaaaagcaagtattattgttatta

SEQ ID NO: 1540 - DNA, 25 bp, Homo sapiens
caaaagcaagtattattgttattat

SEQ ID NO: 1541 - DNA, 25 bp, Homo sapiens
aaaagcaagtattattgttattatg

SEQ ID NO: 1542 - DNA, 25 bp, Homo sapiens
aaagcaagtattattgttattatga

SEQ ID NO: 1543 - DNA, 25 bp, Homo sapiens
aagcaagtattattgttattatgat

SEQ ID NO: 1544 - DNA, 25 bp, Homo sapiens
agcaagtattattgttattatgatt

SEQ ID NO: 1545 - DNA, 25 bp, Homo sapiens
gcaagtattattgttattatgattt

SEQ ID NO: 1546 - DNA, 25 bp, Homo sapiens
caagtattattgttattatgatttg

SEQ ID NO: 1547 - DNA, 25 bp, Homo sapiens
aagtattattgttattatgatttgt

SEQ ID NO: 1548 - DNA, 25 bp, Homo sapiens
agtattattgttattatgatttgta

SEQ ID NO: 1549 - DNA, 25 bp, Homo sapiens
gtattattgttattatgatttgtat

SEQ ID NO: 1550 - DNA, 25 bp, Homo sapiens
tattattgttattatgatttgtatt

SEQ ID NO: 1551 - DNA, 25 bp, Homo sapiens
attattgttattatgatttgtatta

SEQ ID NO: 1552 - DNA, 25 bp, Homo sapiens
ttattgttattatgatttgtattat

SEQ ID NO: 1553 - DNA, 25 bp, Homo sapiens
tattgttattatgatttgtattatt

SEQ ID NO: 1554 - DNA, 25 bp, Homo sapiens
attgttattatgatttgtattatta

SEQ ID NO: 1555 - DNA, 25 bp, Homo sapiens
ttgttattatgatttgtattattat

SEQ ID NO: 1556 - DNA, 25 bp, Homo sapiens
tgttattatgatttgtattattatt

SEQ ID NO: 1557 - DNA, 25 bp, Homo sapiens
gttattatgatttgtattattatta

SEQ ID NO: 1558 - DNA, 25 bp, Homo sapiens
ttattatgatttgtattattattat

SEQ ID NO: 1559 - DNA, 25 bp, Homo sapiens
tattatgatttgtattattattatt

SEQ ID NO: 1560 - DNA, 25 bp, Homo sapiens
attatgatttgtattattattattg

SEQ ID NO: 1561 - DNA, 25 bp, Homo sapiens
ttatgatttgtattattattattgc

SEQ ID NO: 1562 - DNA, 25 bp, Homo sapiens
tatgatttgtattattattattgct

SEQ ID NO: 1563 - DNA, 25 bp, Homo sapiens
atgatttgtattattattattgctg

SEQ ID NO: 1564 - DNA, 25 bp, Homo sapiens
tgatttgtattattattattgctgt

SEQ ID NO: 1565 - DNA, 25 bp, Homo sapiens
gatttgtattattattattgctgtt

SEQ ID NO: 1566 - DNA, 25 bp, Homo sapiens
atttgtattattattattgctgtta

SEQ ID NO: 1567 - DNA, 25 bp, Homo sapiens
tttgtattattattattgctgttac

SEQ ID NO: 1568 - DNA, 25 bp, Homo sapiens
ttgtattattattattgctgttact

SEQ ID NO: 1569 - DNA, 25 bp, Homo sapiens
tgtattattattattgctgttactg

SEQ ID NO: 1570 - DNA, 25 bp, Homo sapiens
gtattattattattgctgttactgt

SEQ ID NO: 1571 - DNA, 25 bp, Homo sapiens
tattattattattgctgttactgtt

SEQ ID NO: 1572 - DNA, 25 bp, Homo sapiens
attattattattgctgttactgtta

SEQ ID NO: 1573 - DNA, 25 bp, Homo sapiens
ttattattattgctgttactgttat

SEQ ID NO: 1574 - DNA, 25 bp, Homo sapiens
tattattattgctgttactgttatt

SEQ ID NO: 1575 - DNA, 25 bp, Homo sapiens
attattattgctgttactgttatta

SEQ ID NO: 1576 - DNA, 25 bp, Homo sapiens
ttattattgctgttactgttattac

SEQ ID NO: 1577 - DNA, 25 bp, Homo sapiens
tattattgctgttactgttattaca

SEQ ID NO: 1578 - DNA, 25 bp, Homo sapiens
attattgctgttactgttattacac

SEQ ID NO: 1579 - DNA, 25 bp, Homo sapiens
ttattgctgttactgttattacacc

SEQ ID NO: 1580 - DNA, 25 bp, Homo sapiens
tattgctgttactgttattacacca

SEQ ID NO: 1581 - DNA, 25 bp, Homo sapiens
attgctgttactgttattacaccaa

SEQ ID NO: 1582 - DNA, 25 bp, Homo sapiens
ttgctgttactgttattacaccaac

SEQ ID NO: 1583 - DNA, 25 bp, Homo sapiens
tgctgttactgttattacaccaact

SEQ ID NO: 1584 - DNA, 25 bp, Homo sapiens
gctgttactgttattacaccaactt

SEQ ID NO: 1585 - DNA, 25 bp, Homo sapiens
ctgttactgttattacaccaacttt

SEQ ID NO: 1586 - DNA, 25 bp, Homo sapiens
tgttactgttattacaccaactttc

SEQ ID NO: 1587 - DNA, 25 bp, Homo sapiens
gttactgttattacaccaactttca

SEQ ID NO: 1588 - DNA, 25 bp, Homo sapiens
ttactgttattacaccaactttcag

SEQ ID NO: 1589 - DNA, 25 bp, Homo sapiens
tactgttattacaccaactttcagg

SEQ ID NO: 1590 - DNA, 25 bp, Homo sapiens
actgttattacaccaactttcagga

SEQ ID NO: 1591 - DNA, 25 bp, Homo sapiens
ctgttattacaccaactttcaggag

SEQ ID NO: 1592 - DNA, 25 bp, Homo sapiens
tgttattacaccaactttcaggagg

SEQ ID NO: 1593 - DNA, 25 bp, Homo sapiens
gttattacaccaactttcaggagga

SEQ ID NO: 1594 - DNA, 25 bp, Homo sapiens
ttattacaccaactttcaggaggat

SEQ ID NO: 1595 - DNA, 25 bp, Homo sapiens
tattacaccaactttcaggaggatg

SEQ ID NO: 1596 - DNA, 25 bp, Homo sapiens
attacaccaactttcaggaggatgt

SEQ ID NO: 1597 - DNA, 25 bp, Homo sapiens
ttacaccaactttcaggaggatgtt

SEQ ID NO: 1598 - DNA, 25 bp, Homo sapiens
tacaccaactttcaggaggatgttt

SEQ ID NO: 1599 - DNA, 25 bp, Homo sapiens
acaccaactttcaggaggatgtttt

SEQ ID NO: 1600 - DNA, 25 bp, Homo sapiens
caccaactttcaggaggatgttttg

SEQ ID NO: 1601 - DNA, 25 bp, Homo sapiens
accaactttcaggaggatgttttgt

SEQ ID NO: 1602 - DNA, 25 bp, Homo sapiens
ccaactttcaggaggatgttttgta

SEQ ID NO: 1603 - DNA, 25 bp, Homo sapiens
caactttcaggaggatgttttgtac

SEQ ID NO: 1604 - DNA, 25 bp, Homo sapiens
aactttcaggaggatgttttgtact

SEQ ID NO: 1605 - DNA, 25 bp, Homo sapiens
actttcaggaggatgttttgtactc

SEQ ID NO: 1606 - DNA, 25 bp, Homo sapiens
ctttcaggaggatgttttgtactcc

SEQ ID NO: 1607 - DNA, 25 bp, Homo sapiens
tttcaggaggatgttttgtactcct

SEQ ID NO: 1608 - DNA, 25 bp, Homo sapiens
ttcaggaggatgttttgtactcctg

SEQ ID NO: 1609 - DNA, 25 bp, Homo sapiens
tcaggaggatgttttgtactcctga

SEQ ID NO: 1610 - DNA, 25 bp, Homo sapiens
caggaggatgttttgtactcctgat

SEQ ID NO: 1611 - DNA, 25 bp, Homo sapiens
aggaggatgttttgtactcctgatg

SEQ ID NO: 1612 - DNA, 25 bp, Homo sapiens
ggaggatgttttgtactcctgatgg

SEQ ID NO: 1613 - DNA, 25 bp, Homo sapiens
gaggatgttttgtactcctgatgga

SEQ ID NO: 1614 - DNA, 25 bp, Homo sapiens
aggatgttttgtactcctgatggaa

SEQ ID NO: 1615 - DNA, 25 bp, Homo sapiens
ggatgttttgtactcctgatggaaa

SEQ ID NO: 1616 - DNA, 25 bp, Homo sapiens
gatgttttgtactcctgatggaaac

SEQ ID NO: 1617 - DNA, 25 bp, Homo sapiens
atgttttgtactcctgatggaaact

SEQ ID NO: 1618 - DNA, 25 bp, Homo sapiens
tgttttgtactcctgatggaaacta

SEQ ID NO: 1619 - DNA, 25 bp, Homo sapiens
gttttgtactcctgatggaaactat

SEQ ID NO: 1620 - DNA, 25 bp, Homo sapiens
ttttgtactcctgatggaaactatt

SEQ ID NO: 1621 - DNA, 25 bp, Homo sapiens
tttgtactcctgatggaaactattg

SEQ ID NO: 1622 - DNA, 25 bp, Homo sapiens
ttgtactcctgatggaaactattgc

SEQ ID NO: 1623 - DNA, 25 bp, Homo sapiens
tgtactcctgatggaaactattgcc

SEQ ID NO: 1624 - DNA, 25 bp, Homo sapiens
gtactcctgatggaaactattgcca

SEQ ID NO: 1625 - DNA, 25 bp, Homo sapiens
tactcctgatggaaactattgccag

SEQ ID NO: 1626 - DNA, 25 bp, Homo sapiens
actcctgatggaaactattgccaga

SEQ ID NO: 1627 - DNA, 25 bp, Homo sapiens
ctcctgatggaaactattgccagac

SEQ ID NO: 1628 - DNA, 25 bp, Homo sapiens
tcctgatggaaactattgccagacc

SEQ ID NO: 1629 - DNA, 25 bp, Homo sapiens
cctgatggaaactattgccagaccc

SEQ ID NO: 1630 - DNA, 25 bp, Homo sapiens
ctgatggaaactattgccagaccca

SEQ ID NO: 1631 - DNA, 25 bp, Homo sapiens
tgatggaaactattgccagacccac

SEQ ID NO: 1632 - DNA, 25 bp, Homo sapiens
gatggaaactattgccagacccaca

SEQ ID NO: 1633 - DNA, 25 bp, Homo sapiens
atggaaactattgccagacccacat

SEQ ID NO: 1634 - DNA, 25 bp, Homo sapiens
tggaaactattgccagacccacatg

SEQ ID NO: 1635 - DNA, 25 bp, Homo sapiens
ggaaactattgccagacccacatgt

SEQ ID NO: 1636 - DNA, 25 bp, Homo sapiens
gaaactattgccagacccacatgta

SEQ ID NO: 1637 - DNA, 25 bp, Homo sapiens
aaactattgccagacccacatgtag

SEQ ID NO: 1638 - DNA, 25 bp, Homo sapiens
aactattgccagacccacatgtagt

SEQ ID NO: 1639 - DNA, 25 bp, Homo sapiens
actattgccagacccacatgtagtc

SEQ ID NO: 1640 - DNA, 25 bp, Homo sapiens
ctattgccagacccacatgtagtca

SEQ ID NO: 1641 - DNA, 25 bp, Homo sapiens
tattgccagacccacatgtagtcaa

SEQ ID NO: 1642 - DNA, 25 bp, Homo sapiens
attgccagacccacatgtagtcaac

SEQ ID NO: 1643 - DNA, 25 bp, Homo sapiens
ttgccagacccacatgtagtcaaca

SEQ ID NO: 1644 - DNA, 25 bp, Homo sapiens
tgccagacccacatgtagtcaacat

SEQ ID NO: 1645 - DNA, 25 bp, Homo sapiens
gccagacccacatgtagtcaacata

SEQ ID NO: 1646 - DNA, 25 bp, Homo sapiens
ccagacccacatgtagtcaacataa

SEQ ID NO: 1647 - DNA, 25 bp, Homo sapiens
cagacccacatgtagtcaacataaa

SEQ ID NO: 1648 - DNA, 25 bp, Homo sapiens
agacccacatgtagtcaacataaac

SEQ ID NO: 1649 - DNA, 25 bp, Homo sapiens
gacccacatgtagtcaacataaacc

SEQ ID NO: 1650 - DNA, 25 bp, Homo sapiens
acccacatgtagtcaacataaaccc

SEQ ID NO: 1651 - DNA, 25 bp, Homo sapiens
cccacatgtagtcaacataaacccc

SEQ ID NO: 1652 - DNA, 25 bp, Homo sapiens
ccacatgtagtcaacataaacccca

SEQ ID NO: 1653 - DNA, 25 bp, Homo sapiens
cacatgtagtcaacataaaccccac

SEQ ID NO: 1654 - DNA, 25 bp, Homo sapiens
acatgtagtcaacataaaccccact

SEQ ID NO: 1655 - DNA, 25 bp, Homo sapiens
catgtagtcaacataaaccccactt

SEQ ID NO: 1656 - DNA, 25 bp, Homo sapiens
atgtagtcaacataaaccccacttt

SEQ ID NO: 1657 - DNA, 25 bp, Homo sapiens
tgtagtcaacataaaccccactttt

SEQ ID NO: 1658 - DNA, 25 bp, Homo sapiens
gtagtcaacataaaccccacttttc

SEQ ID NO: 1659 - DNA, 25 bp, Homo sapiens
tagtcaacataaaccccacttttct

SEQ ID NO: 1660 - DNA, 25 bp, Homo sapiens
agtcaacataaaccccacttttcta

SEQ ID NO: 1661 - DNA, 25 bp, Homo sapiens
gtcaacataaaccccacttttctat

SEQ ID NO: 1662 - DNA, 25 bp, Homo sapiens
tcaacataaaccccacttttctatg

SEQ ID NO: 1663 - DNA, 25 bp, Homo sapiens
caacataaaccccacttttctatgg

SEQ ID NO: 1664 - DNA, 25 bp, Homo sapiens
aacataaaccccacttttctatggc

SEQ ID NO: 1665 - DNA, 25 bp, Homo sapiens
acataaaccccacttttctatggca

SEQ ID NO: 1666 - DNA, 25 bp, Homo sapiens
cataaaccccacttttctatggcaa

SEQ ID NO: 1667 - DNA, 25 bp, Homo sapiens
ataaaccccacttttctatggcaat

SEQ ID NO: 1668 - DNA, 25 bp, Homo sapiens
taaaccccacttttctatggcaatt

SEQ ID NO: 1669 - DNA, 25 bp, Homo sapiens
aaaccccacttttctatggcaattc

SEQ ID NO: 1670 - DNA, 25 bp, Homo sapiens
aaccccacttttctatggcaattca

SEQ ID NO: 1671 - DNA, 25 bp, Homo sapiens
accccacttttctatggcaattcac

SEQ ID NO: 1672 - DNA, 25 bp, Homo sapiens
ccccacttttctatggcaattcact

SEQ ID NO: 1673 - DNA, 25 bp, Homo sapiens
cccacttttctatggcaattcactt

SEQ ID NO: 1674 - DNA, 25 bp, Homo sapiens
ccacttttctatggcaattcacttt

SEQ ID NO: 1675 - DNA, 25 bp, Homo sapiens
cacttttctatggcaattcactttt

SEQ ID NO: 1676 - DNA, 25 bp, Homo sapiens
acttttctatggcaattcacttttt

SEQ ID NO: 1677 - DNA, 25 bp, Homo sapiens
cttttctatggcaattcacttttta

SEQ ID NO: 1678 - DNA, 25 bp, Homo sapiens
ttttctatggcaattcactttttaa

SEQ ID NO: 1679 - DNA, 25 bp, Homo sapiens
tttctatggcaattcactttttaag

SEQ ID NO: 1680 - DNA, 25 bp, Homo sapiens
ttctatggcaattcactttttaaga

SEQ ID NO: 1681 - DNA, 25 bp, Homo sapiens
tctatggcaattcactttttaagag

SEQ ID NO: 1682 - DNA, 25 bp, Homo sapiens
ctatggcaattcactttttaagagt

SEQ ID NO: 1683 - DNA, 25 bp, Homo sapiens
tatggcaattcactttttaagagtc

SEQ ID NO: 1684 - DNA, 25 bp, Homo sapiens
atggcaattcactttttaagagtca

SEQ ID NO: 1685 - DNA, 25 bp, Homo sapiens
tggcaattcactttttaagagtcat

SEQ ID NO: 1686 - DNA, 25 bp, Homo sapiens
ggcaattcactttttaagagtcata

SEQ ID NO: 1687 - DNA, 25 bp, Homo sapiens
gcaattcactttttaagagtcatac

SEQ ID NO: 1688 - DNA, 25 bp, Homo sapiens
caattcactttttaagagtcatact

SEQ ID NO: 1689 - DNA, 25 bp, Homo sapiens
aattcactttttaagagtcatactt

SEQ ID NO: 1690 - DNA, 25 bp, Homo sapiens
attcactttttaagagtcatacttt

SEQ ID NO: 1691 - DNA, 25 bp, Homo sapiens
ttcactttttaagagtcatacttta

SEQ ID NO: 1692 - DNA, 25 bp, Homo sapiens
tcactttttaagagtcatactttat

SEQ ID NO: 1693 - DNA, 25 bp, Homo sapiens
cactttttaagagtcatactttatt

SEQ ID NO: 1694 - DNA, 25 bp, Homo sapiens
actttttaagagtcatactttattt

SEQ ID NO: 1695 - DNA, 25 bp, Homo sapiens
ctttttaagagtcatactttatttt

SEQ ID NO: 1696 - DNA, 25 bp, Homo sapiens
tttttaagagtcatactttattttt

SEQ ID NO: 1697 - DNA, 25 bp, Homo sapiens
ttttaagagtcatactttatttttt

SEQ ID NO: 1698 - DNA, 25 bp, Homo sapiens
tttaagagtcatactttattttttt

SEQ ID NO: 1699 - DNA, 25 bp, Homo sapiens
ttaagagtcatactttatttttttg

SEQ ID NO: 1700 - DNA, 25 bp, Homo sapiens
taagagtcatactttatttttttgc

SEQ ID NO: 1701 - DNA, 25 bp, Homo sapiens
aagagtcatactttatttttttgca

SEQ ID NO: 1702 - DNA, 25 bp, Homo sapiens
agagtcatactttatttttttgcac

SEQ ID NO: 1703 - DNA, 25 bp, Homo sapiens
gagtcatactttatttttttgcaca

SEQ ID NO: 1704 - DNA, 25 bp, Homo sapiens
agtcatactttatttttttgcacag

SEQ ID NO: 1705 - DNA, 25 bp, Homo sapiens
gtcatactttatttttttgcacaga

SEQ ID NO: 1706 - DNA, 25 bp, Homo sapiens
tcatactttatttttttgcacagac

SEQ ID NO: 1707 - DNA, 25 bp, Homo sapiens
catactttatttttttgcacagact

SEQ ID NO: 1708 - DNA, 25 bp, Homo sapiens
atactttatttttttgcacagacta

SEQ ID NO: 1709 - DNA, 25 bp, Homo sapiens
tactttatttttttgcacagactat

SEQ ID NO: 1710 - DNA, 25 bp, Homo sapiens
actttatttttttgcacagactata

SEQ ID NO: 1711 - DNA, 25 bp, Homo sapiens
ctttatttttttgcacagactatat

SEQ ID NO: 1712 - DNA, 25 bp, Homo sapiens
tttatttttttgcacagactatatg

SEQ ID NO: 1713 - DNA, 25 bp, Homo sapiens
ttatttttttgcacagactatatga

SEQ ID NO: 1714 - DNA, 25 bp, Homo sapiens
tatttttttgcacagactatatgag

SEQ ID NO: 1715 - DNA, 25 bp, Homo sapiens
atttttttgcacagactatatgagt

SEQ ID NO: 1716 - DNA, 25 bp, Homo sapiens
tttttttgcacagactatatgagtg

SEQ ID NO: 1717 - DNA, 25 bp, Homo sapiens
ttttttgcacagactatatgagtga

SEQ ID NO: 1718 - DNA, 25 bp, Homo sapiens
tttttgcacagactatatgagtgat

SEQ ID NO: 1719 - DNA, 25 bp, Homo sapiens
ttttgcacagactatatgagtgatg

SEQ ID NO: 1720 - DNA, 25 bp, Homo sapiens
tttgcacagactatatgagtgatgc

SEQ ID NO: 1721 - DNA, 25 bp, Homo sapiens
ttgcacagactatatgagtgatgca

SEQ ID NO: 1722 - DNA, 25 bp, Homo sapiens
tgcacagactatatgagtgatgcat

SEQ ID NO: 1723 - DNA, 25 bp, Homo sapiens
gcacagactatatgagtgatgcatg

SEQ ID NO: 1724 - DNA, 25 bp, Homo sapiens
cacagactatatgagtgatgcatgc

SEQ ID NO: 1725 - DNA, 25 bp, Homo sapiens
acagactatatgagtgatgcatgcc

SEQ ID NO: 1726 - DNA, 25 bp, Homo sapiens
cagactatatgagtgatgcatgcca

SEQ ID NO: 1727 - DNA, 25 bp, Homo sapiens
agactatatgagtgatgcatgccac

SEQ ID NO: 1728 - DNA, 25 bp, Homo sapiens
gactatatgagtgatgcatgccaca

SEQ ID NO: 1729 - DNA, 25 bp, Homo sapiens
actatatgagtgatgcatgccacag

SEQ ID NO: 1730 - DNA, 25 bp, Homo sapiens
ctatatgagtgatgcatgccacagg

SEQ ID NO: 1731 - DNA, 25 bp, Homo sapiens
tatatgagtgatgcatgccacagga

SEQ ID NO: 1732 - DNA, 25 bp, Homo sapiens
atatgagtgatgcatgccacaggag

SEQ ID NO: 1733 - DNA, 25 bp, Homo sapiens
tatgagtgatgcatgccacaggagc

SEQ ID NO: 1734 - DNA, 25 bp, Homo sapiens
atgagtgatgcatgccacaggagct

SEQ ID NO: 1735 - DNA, 25 bp, Homo sapiens
tgagtgatgcatgccacaggagctc

SEQ ID NO: 1736 - DNA, 25 bp, Homo sapiens
gagtgatgcatgccacaggagctcc

SEQ ID NO: 1737 - DNA, 25 bp, Homo sapiens
agtgatgcatgccacaggagctcca

SEQ ID NO: 1738 - DNA, 25 bp, Homo sapiens
gtgatgcatgccacaggagctccac

SEQ ID NO: 1739 - DNA, 25 bp, Homo sapiens
tgatgcatgccacaggagctccacc

SEQ ID NO: 1740 - DNA, 25 bp, Homo sapiens
gatgcatgccacaggagctccaccc

SEQ ID NO: 1741 - DNA, 25 bp, Homo sapiens
atgcatgccacaggagctccacccc

SEQ ID NO: 1742 - DNA, 25 bp, Homo sapiens
tgcatgccacaggagctccacccct

SEQ ID NO: 1743 - DNA, 25 bp, Homo sapiens
gcatgccacaggagctccacccctg

SEQ ID NO: 1744 - DNA, 25 bp, Homo sapiens
catgccacaggagctccacccctga

SEQ ID NO: 1745 - DNA, 25 bp, Homo sapiens
atgccacaggagctccacccctgag

SEQ ID NO: 1746 - DNA, 25 bp, Homo sapiens
tgccacaggagctccacccctgaga

SEQ ID NO: 1747 - DNA, 25 bp, Homo sapiens
gccacaggagctccacccctgagaa

SEQ ID NO: 1748 - DNA, 25 bp, Homo sapiens
ccacaggagctccacccctgagaag

SEQ ID NO: 1749 - DNA, 25 bp, Homo sapiens
cacaggagctccacccctgagaagt

SEQ ID NO: 1750 - DNA, 25 bp, Homo sapiens
acaggagctccacccctgagaagtt

SEQ ID NO: 1751 - DNA, 25 bp, Homo sapiens
caggagctccacccctgagaagttt

SEQ ID NO: 1752 - DNA, 25 bp, Homo sapiens
aggagctccacccctgagaagtttc

SEQ ID NO: 1753 - DNA, 25 bp, Homo sapiens
ggagctccacccctgagaagtttcc

SEQ ID NO: 1754 - DNA, 25 bp, Homo sapiens
gagctccacccctgagaagtttcct

SEQ ID NO: 1755 - DNA, 25 bp, Homo sapiens
agctccacccctgagaagtttcctt

SEQ ID NO: 1756 - DNA, 25 bp, Homo sapiens
gctccacccctgagaagtttccttt

SEQ ID NO: 1757 - DNA, 25 bp, Homo sapiens
ctccacccctgagaagtttcctttt

SEQ ID NO: 1758 - DNA, 25 bp, Homo sapiens
tccacccctgagaagtttccttttc

SEQ ID NO: 1759 - DNA, 25 bp, Homo sapiens
ccacccctgagaagtttccttttcc

SEQ ID NO: 1760 - DNA, 25 bp, Homo sapiens
cacccctgagaagtttccttttcct

SEQ ID NO: 1761 - DNA, 25 bp, Homo sapiens
acccctgagaagtttccttttccta

SEQ ID NO: 1762 - DNA, 25 bp, Homo sapiens
cccctgagaagtttccttttcctag

SEQ ID NO: 1763 - DNA, 25 bp, Homo sapiens
ccctgagaagtttccttttcctagg

SEQ ID NO: 1764 - DNA, 25 bp, Homo sapiens
cctgagaagtttccttttcctaggg

SEQ ID NO: 1765 - DNA, 25 bp, Homo sapiens
ctgagaagtttccttttcctaggga

SEQ ID NO: 1766 - DNA, 25 bp, Homo sapiens
tgagaagtttccttttcctagggac

SEQ ID NO: 1767 - DNA, 25 bp, Homo sapiens
gagaagtttccttttcctagggacc

SEQ ID NO: 1768 - DNA, 25 bp, Homo sapiens
agaagtttccttttcctagggacct

SEQ ID NO: 1769 - DNA, 25 bp, Homo sapiens
gaagtttccttttcctagggacctg

SEQ ID NO: 1770 - DNA, 25 bp, Homo sapiens
aagtttccttttcctagggacctgg

SEQ ID NO: 1771 - DNA, 25 bp, Homo sapiens
agtttccttttcctagggacctggt

SEQ ID NO: 1772 - DNA, 25 bp, Homo sapiens
gtttccttttcctagggacctggtg

SEQ ID NO: 1773 - DNA, 25 bp, Homo sapiens
tttccttttcctagggacctggtgg

SEQ ID NO: 1774 - DNA, 25 bp, Homo sapiens
ttccttttcctagggacctggtggt

SEQ ID NO: 1775 - DNA, 25 bp, Homo sapiens
tccttttcctagggacctggtggtt

SEQ ID NO: 1776 - DNA, 25 bp, Homo sapiens
ccttttcctagggacctggtggtta

SEQ ID NO: 1777 - DNA, 25 bp, Homo sapiens
cttttcctagggacctggtggttaa

SEQ ID NO: 1778 - DNA, 25 bp, Homo sapiens
ttttcctagggacctggtggttaat

SEQ ID NO: 1779 - DNA, 25 bp, Homo sapiens
tttcctagggacctggtggttaatg

SEQ ID NO: 1780 - DNA, 25 bp, Homo sapiens
ttcctagggacctggtggttaatgg

SEQ ID NO: 1781 - DNA, 25 bp, Homo sapiens
tcctagggacctggtggttaatggt

SEQ ID NO: 1782 - DNA, 25 bp, Homo sapiens
cctagggacctggtggttaatggtt

SEQ ID NO: 1783 - DNA, 25 bp, Homo sapiens
ctagggacctggtggttaatggttt

SEQ ID NO: 1784 - DNA, 25 bp, Homo sapiens
tagggacctggtggttaatggtttc

SEQ ID NO: 1785 - DNA, 25 bp, Homo sapiens
agggacctggtggttaatggtttcc

SEQ ID NO: 1786 - DNA, 25 bp, Homo sapiens
gggacctggtggttaatggtttcct

SEQ ID NO: 1787 - DNA, 25 bp, Homo sapiens
ggacctggtggttaatggtttcctc

SEQ ID NO: 1788 - DNA, 25 bp, Homo sapiens
gacctggtggttaatggtttcctct

SEQ ID NO: 1789 - DNA, 25 bp, Homo sapiens
acctggtggttaatggtttcctctg

SEQ ID NO: 1790 - DNA, 25 bp, Homo sapiens
cctggtggttaatggtttcctctgt

SEQ ID NO: 1791 - DNA, 25 bp, Homo sapiens
ctggtggttaatggtttcctctgtt

SEQ ID NO: 1792 - DNA, 25 bp, Homo sapiens
tggtggttaatggtttcctctgtta

SEQ ID NO: 1793 - DNA, 25 bp, Homo sapiens
ggtggttaatggtttcctctgttac

SEQ ID NO: 1794 - DNA, 25 bp, Homo sapiens
gtggttaatggtttcctctgttacc

SEQ ID NO: 1795 - DNA, 25 bp, Homo sapiens
tggttaatggtttcctctgttacct

SEQ ID NO: 1796 - DNA, 25 bp, Homo sapiens
ggttaatggtttcctctgttacctg

SEQ ID NO: 1797 - DNA, 25 bp, Homo sapiens
gttaatggtttcctctgttacctgt

SEQ ID NO: 1798 - DNA, 25 bp, Homo sapiens
ttaatggtttcctctgttacctgtt

SEQ ID NO: 1799 - DNA, 25 bp, Homo sapiens
taatggtttcctctgttacctgttg

SEQ ID NO: 1800 - DNA, 25 bp, Homo sapiens
aatggtttcctctgttacctgttgg

SEQ ID NO: 1801 - DNA, 25 bp, Homo sapiens
atggtttcctctgttacctgttgga

SEQ ID NO: 1802 - DNA, 25 bp, Homo sapiens
tggtttcctctgttacctgttggat

SEQ ID NO: 1803 - DNA, 25 bp, Homo sapiens
ggtttcctctgttacctgttggatt

SEQ ID NO: 1804 - DNA, 25 bp, Homo sapiens
gtttcctctgttacctgttggattg

SEQ ID NO: 1805 - DNA, 25 bp, Homo sapiens
tttcctctgttacctgttggattgc

SEQ ID NO: 1806 - DNA, 25 bp, Homo sapiens
ttcctctgttacctgttggattgcc

SEQ ID NO: 1807 - DNA, 25 bp, Homo sapiens
tcctctgttacctgttggattgcct

SEQ ID NO: 1808 - DNA, 25 bp, Homo sapiens
cctctgttacctgttggattgcctg

SEQ ID NO: 1809 - DNA, 25 bp, Homo sapiens
ctctgttacctgttggattgcctgc

SEQ ID NO: 1810 - DNA, 25 bp, Homo sapiens
tctgttacctgttggattgcctgct

SEQ ID NO: 1811 - DNA, 25 bp, Homo sapiens
ctgttacctgttggattgcctgctc

SEQ ID NO: 1812 - DNA, 25 bp, Homo sapiens
tgttacctgttggattgcctgctca

SEQ ID NO: 1813 - DNA, 25 bp, Homo sapiens
gttacctgttggattgcctgctcaa

SEQ ID NO: 1814 - DNA, 25 bp, Homo sapiens
ttacctgttggattgcctgctcaat

SEQ ID NO: 1815 - DNA, 25 bp, Homo sapiens
tacctgttggattgcctgctcaata

SEQ ID NO: 1816 - DNA, 25 bp, Homo sapiens
acctgttggattgcctgctcaataa

SEQ ID NO: 1817 - DNA, 25 bp, Homo sapiens
cctgttggattgcctgctcaataag

SEQ ID NO: 1818 - DNA, 25 bp, Homo sapiens
ctgttggattgcctgctcaataagt

SEQ ID NO: 1819 - DNA, 25 bp, Homo sapiens
tgttggattgcctgctcaataagta

SEQ ID NO: 1820 - DNA, 25 bp, Homo sapiens
gttggattgcctgctcaataagtat

SEQ ID NO: 1821 - DNA, 25 bp, Homo sapiens
ttggattgcctgctcaataagtatt

SEQ ID NO: 1822 - DNA, 25 bp, Homo sapiens
tggattgcctgctcaataagtattt

SEQ ID NO: 1823 - DNA, 25 bp, Homo sapiens
ggattgcctgctcaataagtatttt

SEQ ID NO: 1824 - DNA, 25 bp, Homo sapiens
gattgcctgctcaataagtattttg

SEQ ID NO: 1825 - DNA, 25 bp, Homo sapiens
attgcctgctcaataagtattttgt

SEQ ID NO: 1826 - DNA, 25 bp, Homo sapiens
ttgcctgctcaataagtattttgtg

SEQ ID NO: 1827 - DNA, 25 bp, Homo sapiens
tgcctgctcaataagtattttgtgc

SEQ ID NO: 1828 - DNA, 25 bp, Homo sapiens
gcctgctcaataagtattttgtgct

SEQ ID NO: 1829 - DNA, 25 bp, Homo sapiens
cctgctcaataagtattttgtgctg

SEQ ID NO: 1830 - DNA, 25 bp, Homo sapiens
ctgctcaataagtattttgtgctgt

SEQ ID NO: 1831 - DNA, 25 bp, Homo sapiens
tgctcaataagtattttgtgctgtg

SEQ ID NO: 1832 - DNA, 25 bp, Homo sapiens
gctcaataagtattttgtgctgtga

SEQ ID NO: 1833 - DNA, 25 bp, Homo sapiens
ctcaataagtattttgtgctgtgac

SEQ ID NO: 1834 - DNA, 25 bp, Homo sapiens
tcaataagtattttgtgctgtgacc

SEQ ID NO: 1835 - DNA, 25 bp, Homo sapiens
caataagtattttgtgctgtgacct

SEQ ID NO: 1836 - DNA, 25 bp, Homo sapiens
aataagtattttgtgctgtgacctt

SEQ ID NO: 1837 - DNA, 25 bp, Homo sapiens
ataagtattttgtgctgtgaccttt

SEQ ID NO: 1838 - DNA, 25 bp, Homo sapiens
taagtattttgtgctgtgacctttg

SEQ ID NO: 1839 - DNA, 25 bp, Homo sapiens
aagtattttgtgctgtgacctttgt

SEQ ID NO: 1840 - DNA, 25 bp, Homo sapiens
agtattttgtgctgtgacctttgtg

SEQ ID NO: 1841 - DNA, 25 bp, Homo sapiens
gtattttgtgctgtgacctttgtgg

SEQ ID NO: 1842 - DNA, 25 bp, Homo sapiens
tattttgtgctgtgacctttgtggg

SEQ ID NO: 1843 - DNA, 25 bp, Homo sapiens
attttgtgctgtgacctttgtgggg

SEQ ID NO: 1844 - DNA, 25 bp, Homo sapiens
ttttgtgctgtgacctttgtgggga

SEQ ID NO: 1845 - DNA, 25 bp, Homo sapiens
tttgtgctgtgacctttgtggggag

SEQ ID NO: 1846 - DNA, 25 bp, Homo sapiens
ttgtgctgtgacctttgtggggagg

SEQ ID NO: 1847 - DNA, 25 bp, Homo sapiens
tgtgctgtgacctttgtggggaggg

SEQ ID NO: 1848 - DNA, 25 bp, Homo sapiens
gtgctgtgacctttgtggggagggg

SEQ ID NO: 1849 - DNA, 25 bp, Homo sapiens
tgctgtgacctttgtggggaggggc

SEQ ID NO: 1850 - DNA, 25 bp, Homo sapiens
gctgtgacctttgtggggaggggca

SEQ ID NO: 1851 - DNA, 25 bp, Homo sapiens
ctgtgacctttgtggggaggggcaa

SEQ ID NO: 1852 - DNA, 25 bp, Homo sapiens
tgtgacctttgtggggaggggcaac

SEQ ID NO: 1853 - DNA, 25 bp, Homo sapiens
gtgacctttgtggggaggggcaact

SEQ ID NO: 1854 - DNA, 25 bp, Homo sapiens
tgacctttgtggggaggggcaactg

SEQ ID NO: 1855 - DNA, 25 bp, Homo sapiens
gacctttgtggggaggggcaactga

SEQ ID NO: 1856 - DNA, 25 bp, Homo sapiens
acctttgtggggaggggcaactgac

SEQ ID NO: 1857 - DNA, 25 bp, Homo sapiens
cctttgtggggaggggcaactgacc

SEQ ID NO: 1858 - DNA, 25 bp, Homo sapiens
ctttgtggggaggggcaactgacca

SEQ ID NO: 1859 - DNA, 25 bp, Homo sapiens
tttgtggggaggggcaactgaccat

SEQ ID NO: 1860 - DNA, 25 bp, Homo sapiens
ttgtggggaggggcaactgaccata

SEQ ID NO: 1861 - DNA, 25 bp, Homo sapiens
tgtggggaggggcaactgaccatat

SEQ ID NO: 1862 - DNA, 25 bp, Homo sapiens
gtggggaggggcaactgaccatata

SEQ ID NO: 1863 - DNA, 25 bp, Homo sapiens
tggggaggggcaactgaccatatac

SEQ ID NO: 1864 - DNA, 25 bp, Homo sapiens
ggggaggggcaactgaccatatact

SEQ ID NO: 1865 - DNA, 25 bp, Homo sapiens
gggaggggcaactgaccatatactt

SEQ ID NO: 1866 - DNA, 25 bp, Homo sapiens
ggaggggcaactgaccatatacttc

SEQ ID NO: 1867 - DNA, 25 bp, Homo sapiens
gaggggcaactgaccatatacttct

SEQ ID NO: 1868 - DNA, 25 bp, Homo sapiens
aggggcaactgaccatatacttctc

SEQ ID NO: 1869 - DNA, 25 bp, Homo sapiens
ggggcaactgaccatatacttctct

SEQ ID NO: 1870 - DNA, 25 bp, Homo sapiens
gggcaactgaccatatacttctcta

SEQ ID NO: 1871 - DNA, 25 bp, Homo sapiens
ggcaactgaccatatacttctctat

SEQ ID NO: 1872 - DNA, 25 bp, Homo sapiens
gcaactgaccatatacttctctatt

SEQ ID NO: 1873 - DNA, 25 bp, Homo sapiens
caactgaccatatacttctctattc

SEQ ID NO: 1874 - DNA, 25 bp, Homo sapiens
aactgaccatatacttctctattct

SEQ ID NO: 1875 - DNA, 25 bp, Homo sapiens
actgaccatatacttctctattcta

SEQ ID NO: 1876 - DNA, 25 bp, Homo sapiens
ctgaccatatacttctctattctag

SEQ ID NO: 1877 - DNA, 25 bp, Homo sapiens
tgaccatatacttctctattctagg

SEQ ID NO: 1878 - DNA, 25 bp, Homo sapiens
gaccatatacttctctattctagga

SEQ ID NO: 1879 - DNA, 25 bp, Homo sapiens
accatatacttctctattctaggaa

SEQ ID NO: 1880 - DNA, 25 bp, Homo sapiens
ccatatacttctctattctaggaat

SEQ ID NO: 1881 - DNA, 25 bp, Homo sapiens
catatacttctctattctaggaata

SEQ ID NO: 1882 - DNA, 25 bp, Homo sapiens
atatacttctctattctaggaatag

SEQ ID NO: 1883 - DNA, 25 bp, Homo sapiens
tatacttctctattctaggaataga

SEQ ID NO: 1884 - DNA, 25 bp, Homo sapiens
atacttctctattctaggaatagat

SEQ ID NO: 1885 - DNA, 25 bp, Homo sapiens
tacttctctattctaggaatagata

SEQ ID NO: 1886 - DNA, 25 bp, Homo sapiens
acttctctattctaggaatagatat

SEQ ID NO: 1887 - DNA, 25 bp, Homo sapiens
cttctctattctaggaatagatata

SEQ ID NO: 1888 - DNA, 25 bp, Homo sapiens
ttctctattctaggaatagatataa

SEQ ID NO: 1889 - DNA, 25 bp, Homo sapiens
tctctattctaggaatagatataaa

SEQ ID NO: 1890 - DNA, 25 bp, Homo sapiens
ctctattctaggaatagatataaaa

SEQ ID NO: 1891 - DNA, 25 bp, Homo sapiens
tctattctaggaatagatataaaag

SEQ ID NO: 1892 - DNA, 25 bp, Homo sapiens
ctattctaggaatagatataaaaga

SEQ ID NO: 1893 - DNA, 25 bp, Homo sapiens
tattctaggaatagatataaaagaa

SEQ ID NO: 1894 - DNA, 25 bp, Homo sapiens
attctaggaatagatataaaagaaa

SEQ ID NO: 1895 - DNA, 25 bp, Homo sapiens
ttctaggaatagatataaaagaaac

SEQ ID NO: 1896 - DNA, 25 bp, Homo sapiens
tctaggaatagatataaaagaaaca

SEQ ID NO: 1897 - DNA, 25 bp, Homo sapiens
ctaggaatagatataaaagaaacat

SEQ ID NO: 1898 - DNA, 25 bp, Homo sapiens
taggaatagatataaaagaaacatt

SEQ ID NO: 1899 - DNA, 25 bp, Homo sapiens
aggaatagatataaaagaaacattt

SEQ ID NO: 1900 - DNA, 25 bp, Homo sapiens
ggaatagatataaaagaaacatttt

SEQ ID NO: 1901 - DNA, 25 bp, Homo sapiens
gaatagatataaaagaaacatttta

SEQ ID NO: 1902 - DNA, 25 bp, Homo sapiens
aatagatataaaagaaacattttag

SEQ ID NO: 1903 - DNA, 25 bp, Homo sapiens
atagatataaaagaaacattttagc

SEQ ID NO: 1904 - DNA, 25 bp, Homo sapiens
tagatataaaagaaacattttagcc

SEQ ID NO: 1905 - DNA, 25 bp, Homo sapiens
agatataaaagaaacattttagcct

SEQ ID NO: 1906 - DNA, 25 bp, Homo sapiens
gatataaaagaaacattttagcctt

SEQ ID NO: 1907 - DNA, 25 bp, Homo sapiens
atataaaagaaacattttagccttt

SEQ ID NO: 1908 - DNA, 25 bp, Homo sapiens
tataaaagaaacattttagcctttt

SEQ ID NO: 1909 - DNA, 25 bp, Homo sapiens
ataaaagaaacattttagccttttt

SEQ ID NO: 1910 - DNA, 25 bp, Homo sapiens
taaaagaaacattttagccttttta

SEQ ID NO: 1911 - DNA, 25 bp, Homo sapiens
aaaagaaacattttagcctttttat

SEQ ID NO: 1912 - DNA, 25 bp, Homo sapiens
aaagaaacattttagcctttttata

SEQ ID NO: 1913 - DNA, 22 bp, Homo sapiens
agcgggactggagaggagtgag

SEQ ID NO: 1914 - DNA, 25 bp, Homo sapiens
ctaggcatccttccaagtcaggtgg

SEQ ID NO: 1915 - DNA, 25 bp, Homo sapiens
ccacctgacttggaaggatgcctag

SEQ ID NO: 1916 - DNA, 24 bp, Homo sapiens
gagctcctgtggcatgcatcactc

SEQ ID NO: 1917 - protein, 125 aa, Artificial
  Consensus sequence of DUF6 motif 1
LIWALYFVFSKKLLERISPLTFTAYRFLIAGILLILLLFLKFGPALLSLGAILALLYLGIFGTALGYLLYFYGLKYVSASKASVLSSLSPVFTLILSVLLLGEKLTLLQLLGIVLILLGVLLILL

SEQ ID NO: 1918 - protein, 130 aa, Homo sapiens
LLICGIGLTSKYLSEDFHANTPVFQSFLNYILLFLVYTTTLAVRQGEENLLAILRRRWWKYMILGLIDLEANYLVVKAYQYTTLTSIQLLDCFVIPVVILLSWFFLLIRYKAVHFIGIVVCILGMGCMVG

SEQ ID NO: 1919 - protein, 138 aa, Methanothermobacter thermautotrophicus
IFFGISATLDKIMLSSMHPVTIGAYTYIIAGIFLFFIRETPLREHVLNAINRKGESEARIKRNDYLILLLTALLSTVIAPLLFLTGLGDTTAVNASLILNVEVLFIILLGYLIFRETLQLKDFLGIVLIILGAVYLLT

SEQ ID NO: 1920 - protein, 126 aa, Rhodobacter capsulatus
LFWAGNFIVARASRGEWPPVALSFGRWSIALIALLPFAWPWLRRDWPLYRRYFGRILLLTLPGVAAFNSLVYWGLQTSTATNGMLLNSTIPLLIMALGALFWGQRLGLWAILGLIVSTLGVAVVIL

SEQ ID NO: 1921 - protein, 128 aa, Bacillus subtilis
IGEALFTLMAKLLSPHISALAISTFVSLFGFLFFLPFALFEASSFDYSVPTVLDWSYVLYYALFVTVLAFYLWYSGVTKVPAGVSGIFTSVLPVSAVILSGVILKEPFEFVHFIGIACVIGGIFVTVI

SEQ ID NO: 1922 - protein, 125 aa, Artificial; identical to SEQ ID NO: 1917
  Consensus sequence of DUF6 motif 2

SEQ ID NO: 1923 - protein, 128 aa, Homo sapiens
TLYGISNVWEEYIIRTLSRVEFLGMIGLFGAFFSGIQLAIMEHKELLKVPWDWQIGLLYVGFSACMFGLYSFMPVVIKKTSATSVNLSLLTADLYSLFCGLFLFHYKFSGLYLLSFFTILIGLVLYSS

SEQ ID NO: 1924 - protein, 115 aa, Archaeoglobus fulgidus
VLMGTVSVFVRNVGGDTLSVTFLRLFFGFLAVLPFCLRDVGRPDRTLLGLAVFNFLTVASYIAAIQSTEVAMAALLLYMAPVYVIPLSVLMGERVEVKTLLALPLGLIGLYLMLT

SEQ ID NO: 1925 - protein, 126 aa, Pyrococcus horikoshii
SMWGTLGIFAKFLYRYNLSTYTIVFYRVIFALIFLFIYLKIKKIPILIPKERLKFYIAYGFFSIFLFYSLYFYTVKISSVSFAVLMLYTAPAYSTILGKLIFNEEINKKKLLAVILVIGGVISLNY

SEQ ID NO: 1926 - protein, 122 aa, Pyrococcus horikoshii
LIGMLLYGLEPVVISYNPVNPISFAFFSAFLASLILLPFANFEEVKVNWKRGLFIGFFGTFLAYLAYSFGTKMSSSVNAALITRTEVLFSFILSYVILGEAISKSRIAYSTLVIIGVVLVMT

(19) United States
(12) Patent Application Publication
(10) Pub. No.: US 2006/0057666 A1
(43) Pub. Date: Mar. 16, 2006

(54) Human g protein coupled receptor

(76) Inventor: Jian Zhang, Beijing (CN)

Correspondence Address: AMERSHAM BIOSCIENCES;PATENT DEPARTMENT, 800 CENTENNIAL AVENUE, PISCATAWAY, NJ, 08855, US

(21) Appl. No.: 10/492,570

(22) PCT Filed: Oct. 11, 2002
(86) PCT No.: PCT/US02/32599

§ 371 Date: Apr. 12, 2004

Related U.S. Application Data
(60) Provisional application No. 60/329,000, filed on Oct. 12, 2001.

(51) Int. Cl. C07K 14/705; C07H 21/04; C12P 21/06

(52) U.S. Cl. 435/69.1; 435/320.1; 435/325; 530/350; 536/23.5

(57) ABSTRACT

The invention provides isolated nucleic acids that encode GPCR-A-1, and fragments thereof, vectors for propagating and expressing GPCR-A-1 nucleic acids, host cells comprising the nucleic acids and vectors of the present invention, proteins, protein fragments, and protein fusions of the novel GPCR-A-1 isoforms, and antibodies thereto. The invention further provides transgenic cells and non-human organisms comprising human GPCR-A-1 nucleic acids, and transgenic cells and non-human organisms with targeted disruption of the endogenous orthologue of the human GPCR-A-1 gene. The invention further provides pharmaceutical formulations of the nucleic acids, proteins, and antibodies of the present invention, and diagnostic, investigational, and therapeutic methods based on the GPCR-A-1 nucleic acids, proteins, and antibodies of the present invention.

### CROSS REFERENCE TO RELATED APPLICATIONS

This application claims priority under 35 U.S.C. § 120 to U.S. provisional application Ser. No. 60/329,000, filed Oct. 12, 2001; the disclosures of which are incorporated herein by reference in their entireties.

### REFERENCE TO SEQUENCE LISTING SUBMITTED ON COMPACT DISC

The present application includes a Sequence Listing filed on a single CD-R disc, provided in duplicate, containing a single file named pto_PB0180.txt, having 287 kilobytes, last modified on Sep. 30, 2002 and recorded Sep. 30, 2002. The Sequence Listing contained in said file on said disc is incorporated herein by reference in its entirety.

### FIELD OF THE INVENTION

The present invention relates to a novel human G protein coupled receptor. More specifically, the invention provides isolated nucleic acid molecules encoding a human G protein coupled receptor, fragments thereof, vectors and host cells comprising isolated nucleic acid molecules encoding a human G protein coupled receptor, a human G protein coupled receptor polypeptides, antibodies, transgenic cells and non-human organisms, and diagnostic, therapeutic, and investigational methods of using the same.

### BACKGROUND OF THE INVENTION

One of the largest gene families in human genome is the superfamily of G protein coupled receptors (GPCRs). GPCRs are integral membrane proteins. Hydrophobicity mapping of the amino acid sequences of G-protein coupled receptors has led to a model of the typical G-protein-coupled receptor as containing seven hydrophobic membrane-spanning regions with the amino terminal on the extracellular side of the membrane and the carboxyl terminal on the intracellular side of the membrane. GPCRs are found in a very wide range of species, and are always involved in signaling from outside the cell to inside the cell. GPCRs control fundamental cellular processes ranging from embryogenesis and neurotransmitter release to cell survival, proliferation, and differentiation. Inglese et al., J. Biol. Chem. 268:23735-23738 (1993).

Most GPCRs detect a periplasmic small molecule or a peptide that upon binding elicits a response felt by the heterotrimeric G protein at the cytosolic side. Heterotrimeric G proteins are composed of α, β, and γ subunits that relay GPCR stimulation to downstream signaling pathways. Gilman, Annu. Rev. Biochem. 56:615-649 (1987). On ligand binding, GPCR undergoes conformation changes of its intracellular loops that promote the exchange of GDP for GTP on Gα and the subsequent dissociation of Gα and Gβγ. Both GTP-bound Gα and free Gβγ propagate the signal through interactions with downstream effectors. Deactivation of GPCR signaling depends on the rate of Gα GTP hydrolysis. Some GPCRs display other activation mechanisms. For example, opsins are activated by a photon, and the thrombin receptor activates itself after its N-terminus is cleaved of by thrombin. Gilman, Annu. Rev. Biochem. 56:615-649 (1987).

There is an increasing awareness that some GPCRs can regulate proliferative signaling pathways and that chronic stimulation or mutational activation of receptors can lead to oncogenic transformation. Activating mutations in GPCRs are associated with several types of human tumors and some receptors exhibit potent oncogenic activity due to agonist overexpression. Additionally, expression screening analyses for novel oncogenes identified GPCRs whose expression causes the oncogenic transformation of NIH3T3 mouse fibroblasts. These include Mas, G2A, and the PAR-1 thrombin receptor. Whitehead et al., Oncogene 20:1547-1555 (2001); Ryder et al., J. Cell Physiol. 186:53-64 (2001). GPCR signaling pathway has recently also been shown to be involved in tumor metastasis. For example, KiSS, a ligand of a GPCR has been shown to suppress metastases of human melanomas and breast carcinomas. Ohtaki et al., Nature 411: 613-617 (2001); Muir et al., J. Biol. Chem. 276:28969-28975 (2001). GPCRs have also been identified to expression in tumor endothelium in humans and mice, which makes these genes attractive targets for the development of antiangiogenic therapies. Carson-Walter et al., Cancer Res. 61:6649-6655 (2001).

The number of diseases that are caused by a GPCR malfunction is enormous. As a class, GPCRs are historically the single richest receptor target for drug discovery, involved in the development of nearly 60 percent of all prescription drugs on the market today. However, the potential for GPCR-based drugs remains largely untapped due to the fact that only about 200 of these important drug targets are functionally known. It is estimated that a further 1,000 GPCRs are “orphans” (functionally unknown). Decipher the functions of such orphans will provide an array of novel targets for new medicines.

Given the important role of GPCRs in signal transduction, their involvement in tumorigenesis and tumor metastasis, and their proven clinical utility as drug targets, there is a need to identify and to characterize additional human GPCR proteins.

### SUMMARY OF THE INVENTION

The present invention solves these and other needs in the art by providing isolated nucleic acids that encode a human G protein coupled receptor (GPCR-A-1), and fragments thereof.

In other aspects, the invention provides vectors for propagating and expressing the nucleic acids of the present invention, host cells comprising the nucleic acids and vectors of the present invention, proteins, protein fragments, and protein fusions of the GPCR-A-1, and antibodies thereto.

The invention further provides pharmaceutical formulations of the nucleic acids, proteins, and antibodies of the present invention.

In other aspects, the invention provides transgenic cells and non-human organisms comprising GPCR-A-1 nucleic acids, and transgenic cells and non-human organisms with targeted disruption of the endogenous orthologue of the GPCR-A-1.

The invention additionally provides diagnostic, investigational, and therapeutic methods based on the GPCR-A-1 nucleic acids, proteins, and antibodies of the present invention.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 (A) schematizes the protein domain structure of GPCR-A-1, FIG. 1 (B) shows the alignment of the first DUF6 motif of GPCR-A-1 with that of other membrance proteins, and FIG. 1 (C) shows the alignment of the second DUF6 motif of GPCR-A-1 with that of other membrance proteins;

FIG. 2 is a map showing the genomic structure of GPCR-A-1 encoded at chromosome 6q22.1;

FIG. 3 presents the nucleotide and predicted amino acid sequences of GPCR-A-1; and

FIG. 4 shows the expression profile of GPCR-A-1 by RT-PCR.

### DETAILED DESCRIPTION OF THE INVENTION

Mining the sequence of the human genome for novel human genes, the present inventor has identified GPCR-A-1, a G protein coupled receptor functioning as a potential tumor suppressor in colon epithelium, which could be used as a target for both diagnosis and treatment of cancer.

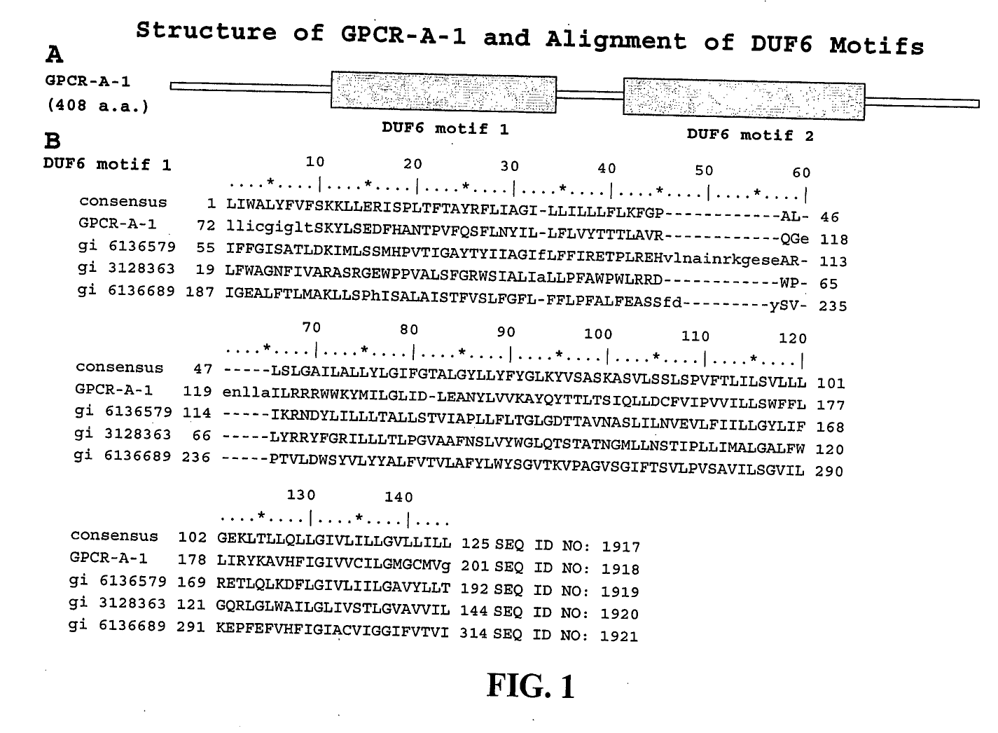

As schematized in FIG. 1, the newly isolated gene product shares certain protein domains and an overall structural organization with other G protein coupled receptors (GPCRs). The shared structural features strongly imply that GPCR-A-1 plays a role similar to that of other G protein coupled receptors in signal transduction, aberrant expression of which could lead to tumorigenesis and tumor metastasis.

Like other GPCRs, GPCR-A-1 has two DUF6 motifs (http://www.ncbi.nlm.nih.gov/Structure/cdd/wrpsb.cgi) and seven transmembrane domains (http://smart.embl-heidelberg.de/smart/show_motifs.pl). The two DUF6 motifs in GPCR-A-1 cover amino acid sequences 81-200 and 229-354 of GPCR-A-1, respectively. DUF6 is a domain often found in integral membrane proteins.

Other signatures of the newly isolated GPCR-A-1 proteins were identified by searching the PROSITE database (http://www.expasy.ch/tools/scnpsitl.html). These include one N-glycosylation sites (313-316), one cAMP- and cGMP-dependent protein kinase phosphorylation site (305-308), two protein kinase C phosphorylation sites (43-45 and 80-82), three Casein kinase II phosphorylation sites (24-27, 245-248, and 399-402), and multiple N-myristoylation sites.

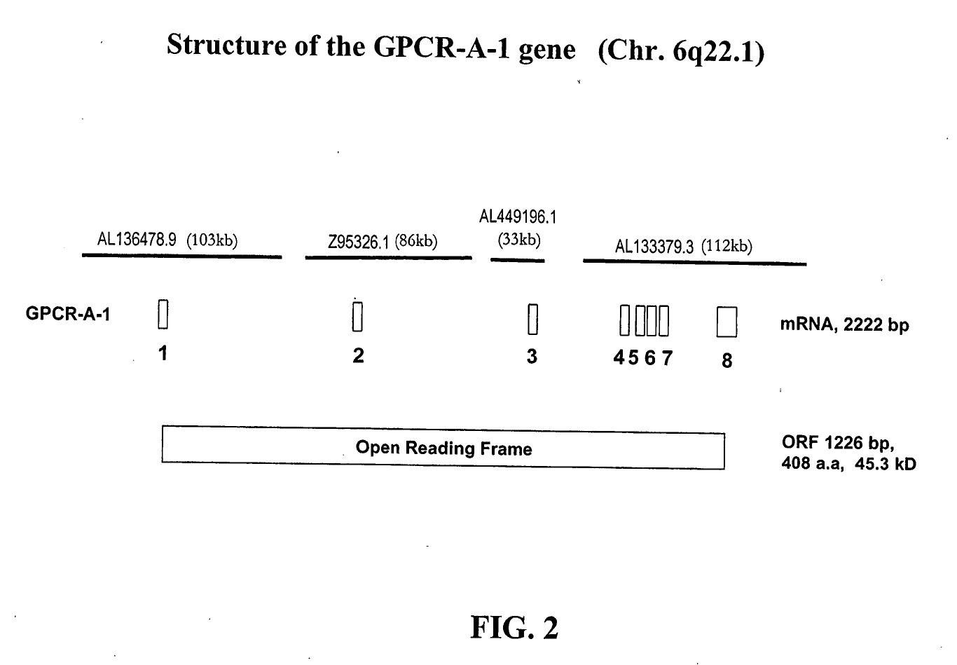

FIG. 2 shows the genomic organization of GPCR-A-1.

At the top is shown the four bacterial artificial chromosomes (BACs), with GenBank accession numbers (AL136478.9, Z95326.1, AL449196.1, AL133379.3), that span the GPCR-A-1 locus.

As shown in FIG. 2, GPCR-A-1, encoding a protein of 408 amino acids, comprises exons 1-8. Predicted molecular weight, prior to any post-translational modification, is 45.3 kD. The cDNA clone appears full length, with the open reading frame starting with a methionine and terminating with a stop codon.

As further discussed in the examples herein, expression of GPCR-A-1 was assessed using RT-PCR. RT-PCR detected high level expression of GPCR-A-1 in brain, heart, colon, testis and uterus, as well as lower expression in kidney, bone marrow, skeletal muscle and placenta.

As more fully described below, the present invention provides isolated nucleic acids that encode GPCR-A-1 and fragments thereof. The invention further provides vectors for propagation and expression of the nucleic acids of the present invention, host cells comprising the nucleic acids and vectors of the present invention, proteins, protein fragments, and protein fusions of the present invention, and antibodies specific for all or any one of the isoforms. The invention provides pharmaceutical formulations of the nucleic acids, proteins, and antibodies of the present invention. The invention further provides transgenic cells and non-human organisms comprising human GPCR-A-1 nucleic acids, and transgenic cells and non-human organisms with targeted disruption of the endogenous orthologue of the human GPCR-A-1. The invention additionally provides diagnostic, investigational, and therapeutic methods based on the GPCR-A-1 nucleic acids, proteins, and antibodies of the present invention.

Definitions

Unless defined otherwise, all technical and scientific terms used herein have the meaning commonly understood by one of ordinary skill in the art to which this invention belongs.

As used herein, “nucleic acid” (synonymously, “polynucleotide”) includes polynucleotides having natural nucleotides in native 5′-3′ phosphodiester linkage—e.g., DNA or RNA—as well as polynucleotides that have nonnatural nucleotide analogues, normative internucleoside bonds, or both, so long as the nonnatural polynucleotide is capable of sequence-discriminating basepairing under experimentally desired conditions. Unless otherwise specified, the term “nucleic acid” includes any topological conformation; the term thus explicitly comprehends single-stranded, double-stranded, partially duplexed, triplexed, hairpinned, circular, and padlocked conformations.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment.

For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature.

As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

As used herein, an isolated nucleic acid “encodes” a reference polypeptide when at least a portion of the nucleic acid, or its complement, can be directly translated to provide the amino acid sequence of the reference polypeptide, or when the isolated nucleic acid can be used, alone or as part of an expression vector, to express the reference polypeptide in vitro, in a prokaryotic host cell, or in a eukaryotic host cell.

As used herein, the term “exon” refers to a nucleic acid sequence found in genomic DNA that is bioinformatically predicted and/or experimentally confirmed to contribute contiguous sequence to a mature mRNA transcript.

As used herein, the phrase “open reading frame” and the equivalent acronym “ORF” refer to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence intends all nucleic acid sequences that can be directly translated, using the standard genetic code, to provide an amino acid sequence identical to that translated from the reference nucleic acid sequence.

As used herein, the term “microarray” and the equivalent phrase “nucleic acid microarray” refer to a substrate-bound collection of plural nucleic acids, hybridization to each of the plurality of bound nucleic acids being separately detectable. The substrate can be solid or porous, planar or non-planar, unitary or distributed.

As so defined, the term “microarray” and phrase “nucleic acid microarray” include all the devices so called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376), the disclosures of which are incorporated herein by reference in their entireties.

As so defined, the term “microarray” and phrase “nucleic acid microarray” also include substrate-bound collections of plural nucleic acids in which the plurality of nucleic acids are distributably disposed on a plurality of beads, rather than on a unitary planar substrate, as is described, inter alia, in Brenner et al., Proc. Natl. Acad. Sci. USA 97(4):166501670 (2000), the disclosure of which is incorporated herein by reference in its entirety; in such case, the term “microarray” and phrase “nucleic acid microarray” refer to the plurality of beads in aggregate.

As used herein with respect to solution phase hybridization, the term “probe”, or equivalently, “nucleic acid probe” or “hybridization probe”, refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to nucleic acid intended to be bound to probe by sequence complementarity.

As used herein, the expression “probe comprising SEQ ID NO:X”, and variants thereof, intends a nucleic acid probe, at least a portion of which probe has either (i) the sequence directly as given in the referenced SEQ ID NO:X, or (ii) a sequence complementary to the sequence as given in the referenced SEQ ID NO:X, the choice as between sequence directly as given and complement thereof dictated by the requirement that the probe be complementary to the desired target.

As used herein, the phrases “expression of a probe” and “expression of an isolated nucleic acid” and their linguistic equivalents intend that the probe or, (respectively, the isolated nucleic acid), or a probe (or, respectively, isolated nucleic acid) complementary in sequence thereto, can hybridize detectably under high stringency conditions to a sample of nucleic acids that derive from mRNA transcripts from a given source. For example, and by way of illustration only, expression of a probe in “liver” means that the probe can hybridize detectably under high stringency conditions to a sample of nucleic acids that derive from mRNA obtained from liver.

As used herein, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and that consist of one or more exons that are found adjacent to the reference exon in the genome.

For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for at least 8 hours, followed by one or more washes in 0.2×SSC, 0.1% SDS at 65° C. “Moderate stringency conditions” are defined for solution phase hybridization as aqueous hybridization (i.e., free of formamide) in 6×SSC, 1% SDS at 65° C. for at least 8 hours, followed by one or more washes in 2×SSC, 0.1% SDS at room temperature.

For microarray-based hybridization, standard “high stringency conditions” are defined as hybridization in 50% formamide, 5×SSC, 0.2 μg/μl poly(dA), 0.2 μg/μl human cot1 DNA, and 0.5% SDS, in a humid oven at 42° C. overnight, followed by successive washes of the microarray in 1×SSC, 0.2% SDS at 55° C. for 5 minutes, and then 0.1×SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein”, “polypeptide”, and “peptide” are used interchangeably to refer to a naturally-occurring or synthetic polymer of amino acid monomers (residues), irrespective of length, where amino acid monomer here includes naturally-occurring amino acids, naturally-occurring amino acid structural variants, and synthetic non-naturally occurring analogs that are capable of participating in peptide bonds. The terms “protein”, “polypeptide”, and “peptide” explicitly permits of post-translational and post-synthetic modifications, such as glycosylation.

The term “oligopeptide” herein denotes a protein, polypeptide, or peptide having 25 or fewer monomeric subunits.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment.

For example, a protein can be said to be “isolated” when it includes amino acid analogues or derivatives not found in nature, or includes linkages other than standard peptide bonds.

When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

A “purified protein” (equally, a purified polypeptide, peptide, or oligopeptide) is an isolated protein, as above described, present at a concentration of at least 95%, as measured on a weight basis with respect to total protein in a composition. A “substantially purified protein” (equally, a substantially purified polypeptide, peptide, or oligopeptide) is an isolated protein, as above described, present at a concentration of at least 70%, as measured on a weight basis with respect to total protein in a composition.

As used herein, the phrase “protein isoforms” refers to a plurality of proteins having nonidentical primary amino acid sequence but that share amino acid sequence encoded by at least one common exon.

As used herein, the phrase “alternative splicing” and its linguistic equivalents includes all types of RNA processing that lead to expression of plural protein isoforms from a single gene; accordingly, the phrase “splice variant(s)” and its linguistic equivalents embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region (“5′ UT”) and alternatives in the 3′ untranslated region (“3′ UT”). Such 3′ alternatives include, for example, differences in the site of RNA transcript cleavage and site of poly(A) addition. See, e.g., Gautheret et al., Genome Res. 8:524-530 (1998).

As used herein, “orthologues” are separate occurrences of the same gene in multiple species. The separate occurrences have similar, albeit nonidentical, amino acid sequences, the degree of sequence similarity depending, in part, upon the evolutionary distance of the species from a common ancestor having the same gene.

As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the term “homologues” is generic to “orthologues” and “paralogues”.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

As used herein, antibodies can be produced by any known technique, including harvest from cell culture of native B lymphocytes, harvest from culture of hybridomas, recombinant expression systems, and phage display.

As used herein, “antigen” refers to a ligand that can be bound by an antibody; an antigen need not itself be immunogenic. The portions of the antigen that make contact with the antibody are denominated “epitopes”.

“Specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, and biotin to any of avidin, streptavidin, neutrAvidin and captAvidin.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “portion”, as used with respect to nucleic acids, proteins, and antibodies, is synonymous with “fragment”.

Nucleic Acid Molecules

In a first aspect, the invention provides isolated nucleic acids that encode GPCR-A-1, variants having at least 65% sequence identity thereto, degenerate variants thereof, variants that encode GPCR-A-1 proteins having conservative or moderately conservative substitutions, cross-hybridizing nucleic acids, and fragments thereof.

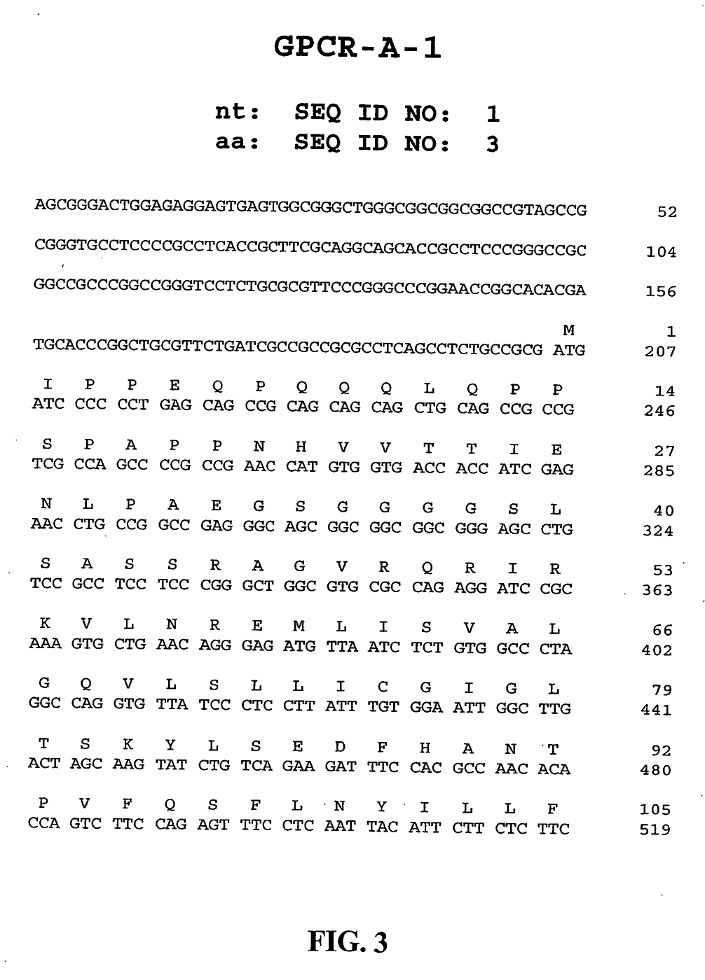

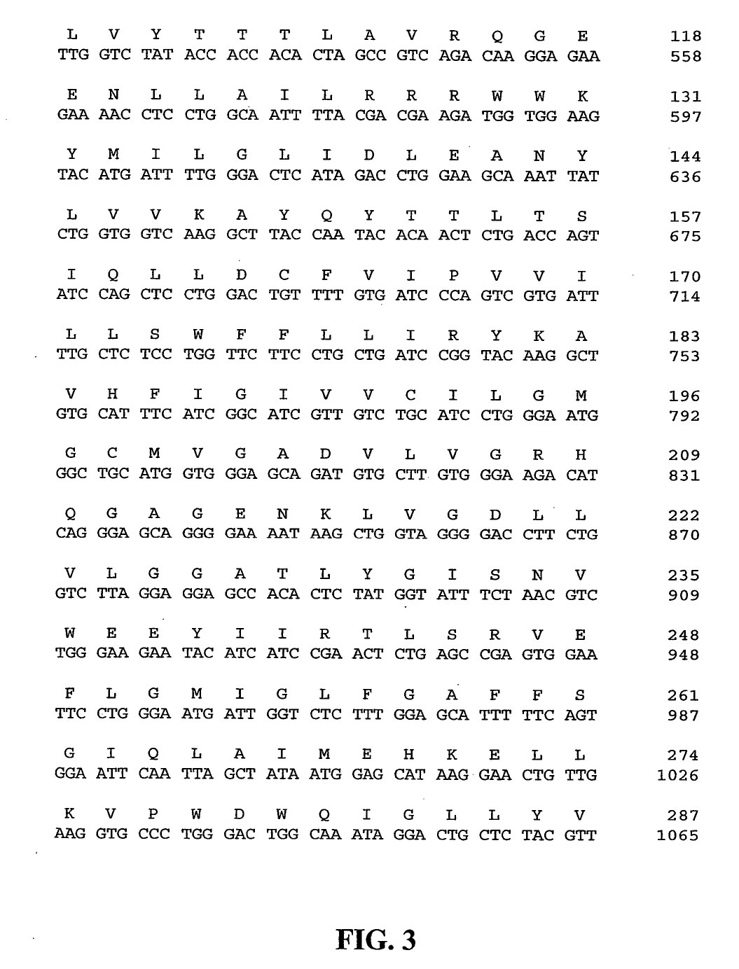

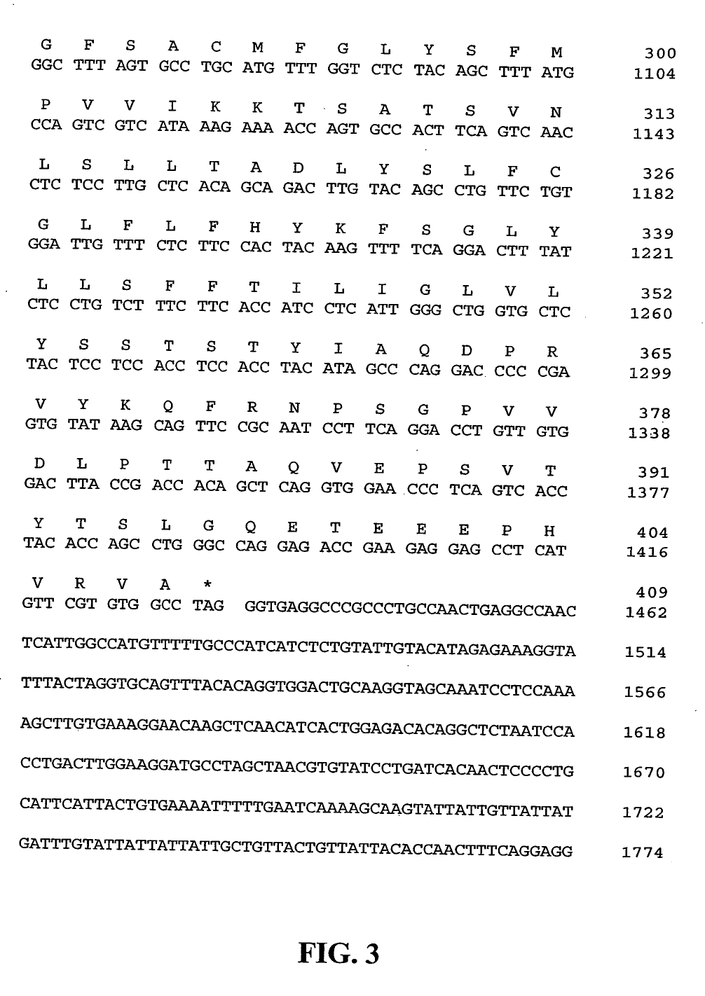

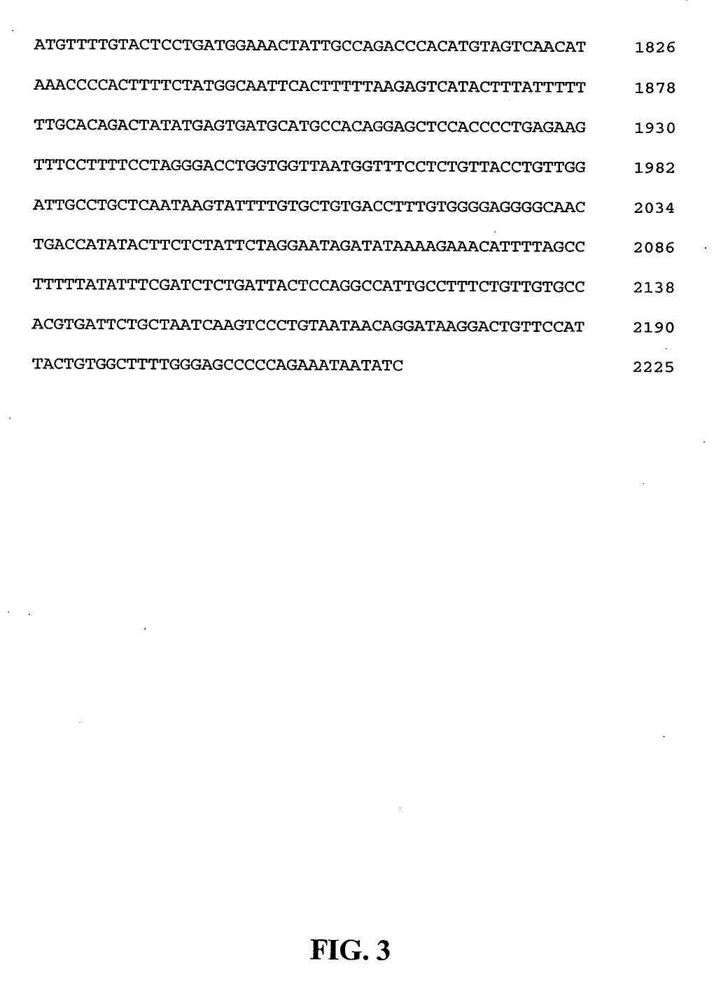

FIG. 3 presents the nucleotide sequence of the GPCR-A-1 cDNA clone, with predicted amino acid translation; the sequences are further presented in the Sequence Listing, incorporated herein by reference in its entirety, in SEQ ID NOs: 1 (full length nucleotide sequence of human GPCR-A-1 cDNA) and 3 (full length amino acid coding sequence of human GPCR-A-1).

Unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Unless otherwise indicated, nucleotide sequences of the isolated nucleic acids of the present invention were determined by sequencing a DNA molecule that had resulted, directly or indirectly, from at least one enzymatic polymerization reaction (e.g., reverse transcription and/or polymerase chain reaction) using an automated sequencer (such as the MegaBACE™ 1000, Amersham Biosciences, Sunnyvale, Calif., USA), or by reliance upon such sequence or upon genomic sequence prior-accessioned into a public database. Unless otherwise indicated, all amino acid sequences of the polypeptides of the present invention were predicted by translation from the nucleic acid sequences so determined.

As a consequence, any nucleic acid sequence presented herein may contain errors introduced by erroneous incorporation of nucleotides during polymerization, by erroneous base calling by the automated sequencer (although such sequencing errors have been minimized for the nucleic acids directly determined herein, unless otherwise indicated, by the sequencing of each of the complementary strands of a duplex DNA), or by similar errors accessioned into the public database. Such errors can readily be identified and corrected by resequencing of the genomic locus using standard techniques.

Single nucleotide polymorphisms (SNPs) occur frequently in eukaryotic genomes—more than 1.4 million SNPs have already identified in the human genome, International Human Genome Sequencing Consortium, Nature 409:860-921 (2001)- and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is an aspect of the present invention to provide nucleic acids not only identical in sequence to those described with particularity herein, but also to provide isolated nucleic acids at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

For purposes herein, percent identity of two nucleic acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at
http://www.ncbi.nlm.nih.gov/blast/bl2seq/bl2.html.
   To assess percent identity of nucleic acids, the BLASTN module of BLAST 2 SEQUENCES is used with default values of (i) reward for a match: 1; (ii) penalty for a mismatch: −2; (iii) open gap 5 and extension gap 2 penalties; (iv) gap X_dropoff 50 expect 10 word size 11 filter, and both sequences are entered in their entireties.

As is well known, the genetic code is degenerate, with each amino acid except methionine translated from a plurality of codons, thus permitting a plurality of nucleic acids of disparate sequence to encode the identical protein. As is also well known, codon choice for optimal expression varies from species to species. The isolated nucleic acids of the present invention being useful for expression of GPCR-A-1 proteins and protein fragments, it is, therefore, another aspect of the present invention to provide isolated nucleic acids that encode GPCR-A-1 proteins and portions thereof not only identical in sequence to those described with particularity herein, but degenerate variants thereof as well.

As is also well known, amino acid substitutions occur frequently among natural allelic variants, with conservative substitutions often occasioning only de minimis change in protein function.

Accordingly, it is an aspect of the present invention to provide nucleic acids not only identical in sequence to those described with particularity herein, but also to provide isolated nucleic acids that encode GPCR-A-1, and portions thereof, having conservative amino acid substitutions, and also to provide isolated nucleic acids that encode GPCR-A-1, and portions thereof, having moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

$$\begin{matrix} & A & R & N & D & C & Q & E & G & H & I & L & K & M & F & P & S & T & W & Y & V \\ A & 2 & - 1 & 0 & 0 & 0 & 0 & 0 & 0 & - 1 & - 1 & - 1 & 0 & - 1 & - 2 & 0 & 1 & 1 & - 4 & - 2 & 0 \\ R & - 1 & 5 & 0 & 0 & - 2 & 2 & 0 & - 1 & 1 & - 2 & - 2 & 3 & - 2 & - 3 & - 1 & 0 & 0 & - 2 & - 2 & - 2 \\ N & 0 & 0 & 4 & 2 & - 2 & 1 & 1 & 0 & 1 & - 3 & - 3 & 1 & - 2 & - 3 & - 1 & 1 & 0 & - 4 & - 1 & - 2 \\ D & 0 & 0 & 2 & 5 & - 3 & 1 & 3 & 0 & 0 & - 4 & - 4 & 0 & - 3 & - 4 & - 1 & 0 & 0 & - 5 & - 3 & - 3 \\ C & 0 & - 2 & - 2 & - 3 & 12 & - 2 & - 3 & - 2 & - 1 & - 1 & - 2 & - 3 & - 1 & - 1 & - 3 & 0 & 0 & - 1 & 0 & 0 \\ Q & 0 & \cdot 2 & 1 & 1 & - 2 & 3 & 2 & - 1 & 1 & - 2 & - 2 & 2 & - 1 & - 3 & 0 & 0 & 0 & - 3 & - 2 & - 2 \\ E & 0 & 0 & 1 & 3 & - 3 & 2 & 4 & - 1 & 0 & - 3 & - 3 & 1 & - 2 & - 4 & 0 & 0 & 0 & - 4 & - 3 & - 2 \\ G & 0 & - 1 & 0 & 0 & - 2 & - 1 & - 1 & 7 & - 1 & - 4 & - 4 & - 1 & - 4 & - 5 & - 2 & 0 & - 1 & - 4 & - 4 & - 3 \\ H & - 1 & 1 & 1 & 0 & - 1 & 1 & 0 & - 1 & 6 & - 2 & - 2 & 1 & - 1 & 0 & - 1 & 0 & 0 & - 1 & 2 & - 2 \\ I & - 1 & - 2 & - 3 & - 4 & - 1 & - 2 & - 3 & - 4 & - 2 & 4 & 3 & - 2 & 2 & 1 & - 3 & - 2 & - 1 & - 2 & - 1 & 3 \\ L & - 1 & - 2 & - 3 & - 4 & - 2 & - 2 & - 3 & - 4 & - 2 & 3 & 4 & - 2 & 3 & 2 & - 2 & - 2 & - 1 & - 1 & 0 & 2 \\ K & 0 & 3 & 1 & 0 & - 3 & 2 & 1 & - 1 & 1 & - 2 & - 2 & 3 & - 1 & - 3 & - 1 & 0 & 0 & - 4 & - 2 & - 2 \\ M & - 1 & - 2 & - 2 & - 3 & - 1 & - 1 & - 2 & - 4 & - 1 & 2 & 3 & - 1 & 4 & 2 & - 2 & - 1 & - 1 & - 1 & 0 & 2 \\ F & - 2 & - 3 & - 3 & - 4 & - 1 & - 3 & - 4 & - 5 & 0 & 1 & 2 & - 3 & 2 & 7 & - 4 & - 3 & - 2 & 4 & 5 & 0 \\ P & 0 & - 1 & - 1 & - 1 & - 3 & 0 & 0 & - 2 & - 1 & - 3 & - 2 & - 1 & - 2 & - 4 & 8 & 0 & 0 & - 5 & - 3 & - 2 \\ S & 1 & 0 & 1 & 0 & 0 & 0 & 0 & 0 & 0 & - 2 & - 2 & 0 & - 1 & - 3 & 0 & 2 & 2 & - 3 & - 2 & - 1 \\ T & 1 & 0 & 0 & 0 & 0 & 0 & 0 & - 1 & 0 & - 1 & - 1 & 0 & - 1 & - 2 & 0 & 2 & 2 & - 4 & - 2 & 0 \\ W & - 4 & - 2 & - 4 & - 5 & - 1 & - 3 & - 4 & - 4 & - 1 & - 2 & - 1 & - 4 & - 1 & 4 & - 5 & - 3 & - 4 & 14 & 4 & - 3 \\ Y & - 2 & - 2 & - 1 & - 3 & 0 & - 2 & - 3 & - 4 & 2 & - 1 & 0 & - 2 & 0 & 5 & - 3 & - 2 & - 2 & 4 & 8 & - 1 \\ V & 0 & - 2 & - 2 & - 3 & 0 & - 2 & - 2 & - 3 & - 2 & 3 & 2 & - 2 & 2 & 0 & - 2 & - 1 & 0 & - 3 & - 1 & 3\end{matrix}$$

For purposes herein, a “moderately conservative” replacement is any change having a nonnegative value in the PAM250 log-likelihood matrix reproduced herein above.

As is also well known in the art, relatedness of nucleic acids can also be characterized using a functional test, the ability of the two nucleic acids to base-pair to one another at defined hybridization stringencies.

It is, therefore, another aspect of the invention to provide isolated nucleic acids not only identical in sequence to those described with particularity herein, but also to provide isolated nucleic acids (“cross-hybridizing nucleic acids”) that hybridize under high stringency conditions (as defined herein below) to all or to a portion of various of the isolated GPCR-A-1 nucleic acids of the present invention (“reference nucleic acids”), as well as cross-hybridizing nucleic acids that hybridize under moderate stringency conditions to all or to a portion of various of the isolated GPCR-A-1 nucleic acids of the present invention.

Such cross-hybridizing nucleic acids are useful, inter alia, as probes for, and to drive expression of, proteins related to the proteins of the present invention as alternative isoforms, homologues, paralogues, and orthologues. Particularly useful orthologues are those from other primate species, such as chimpanzee, rhesus macaque, monkey, baboon, orangutan, and gorilla; from rodents, such as rats, mice, guinea pigs; from lagomorphs, such as rabbits; and from domestic livestock, such as cow, pig, sheep, horse, goat and chicken.

For purposes herein, high stringency conditions are defined as aqueous hybridization (i.e., free of formamide) in 6×SSC (where 20×SSC contains 3.0 M NaCl and 0.3 M sodium citrate), 1% SDS at 65° C. for at least 8 hours, followed by one or more washes in 0.2×SSC, 0.1% SDS at 65° C. For purposes herein, moderate stringency conditions are defined as aqueous hybridization (i.e., free of formamide) in 6×SSC, 1% SDS at 65° C. for at least 8 hours, followed by one or more washes in 2×SSC, 0.1% SDS at room temperature.

The hybridizing portion of the reference nucleic acid is typically at least 15 nucleotides in length, often at least 17 nucleotides in length. Often, however, the hybridizing portion of the reference nucleic acid is at least 20 nucleotides in length, 25 nucleotides in length, and even 30 nucleotides, 35 nucleotides, 40 nucleotides, and 50 nucleotides in length. Of course, cross-hybridizing nucleic acids that hybridize to a larger portion of the reference nucleic acid—for example, to a portion of at least 50 nt, at least 100 nt, at least 150 nt, 200 nt, 250 nt, 300 nt, 350 nt, 400 nt, 450 nt, or 500 nt or more—or even to the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the isolated nucleic acids of the present invention.

By “fragments” of a reference nucleic acid is here intended isolated nucleic acids, however obtained, that have a nucleotide sequence identical to a portion of the reference nucleic acid sequence, which portion is at least 17 nucleotides and less than the entirety of the reference nucleic acid. As so defined, “fragments” need not be obtained by physical fragmentation of the reference nucleic acid, although such provenance is not thereby precluded.

In theory, an oligonucleotide of 17 nucleotides is of sufficient length as to occur at random less frequently than once in the three gigabase human genome, and thus to provide a nucleic acid probe that can uniquely identify the reference sequence in a nucleic acid mixture of genomic complexity. As is well known, further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

As further described herein below, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., “Use of peptide synthesis to probe viral antigens for epitopes to a resolution of a single amino acid,” Proc. Natl. Acad. Sci. USA 81:3998-4002 (1984); and U.S. Pat. Nos. 4,708,871 and 5,595,915, the disclosures of which are incorporated herein by reference in their entireties.

As further described herein below, fragments that encode at least 8 contiguous amino acids (i.e., fragments of 24 nucleotides or more) are useful in directing the expression or the synthesis of peptides that have utility as immunogens. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties.

The nucleic acid fragment of the present invention is thus at least 17 nucleotides in length, typically at least 18 nucleotides in length, and often at least 24 nucleotides in length. Often, the nucleic acid of the present invention is at least 25 nucleotides in length, and even 30 nucleotides, 35 nucleotides, 40 nucleotides, or 45 nucleotides in length. Of course, larger fragments having at least 50 nt, at least 100 nt, at least 150 nt, 200 nt, 250 nt, 300 nt, 350 nt, 400 nt, 450 nt, or 500 nt or more are also useful, and at times preferred.

Having been based upon the mining of genomic sequence, rather than upon surveillance of expressed message, the present invention further provides isolated genome-derived nucleic acids that include portions of the GPCR-A-1 gene.

The invention particularly provides genome-derived single exon probes.

As further described in commonly owned and copending U.S. patent application Ser. No. 09/864,761, filed May 23, 2001; Ser. No. 09/774,203, filed Jan. 29, 2001; and Ser. No. 09/632,366, filed Aug. 3, 2000, the disclosures of which are incorporated herein by reference in their entireties, “a single exon probe” comprises at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon and instead consist of one or more exons that are found adjacent to the reference exon in the genome.

Genome-derived single exon probes typically further comprise, contiguous to a first end of the exon portion, a first intronic and/or intergenic sequence that is identically contiguous to the exon in the genome. Often, the genome-derived single exon probe further comprises, contiguous to a second end of the exonic portion, a second intronic and/or intergenic sequence that is identically contiguous to the exon in the genome.

The minimum length of genome-derived single exon probes is defined by the requirement that the exonic portion be of sufficient length to hybridize under high stringency conditions to transcript-derived nucleic acids. Accordingly, the exon portion is at least 17 nucleotides, typically at least 18 nucleotides, 20 nucleotides, 24 nucleotides, 25 nucleotides or even 30, 35, 40, 45, or 50 nucleotides in length, and can usefully include the entirety of the exon, up to 100 nt, 150 nt, 200 nt, 250 nt, 300 nt, 350 nt, 400 nt or even 500 nt or more in length.

The maximum length of genome-derived single exon probes is defined by the requirement that the probes contain portions of no more than one exon, that is, be unable to hybridize detectably under high stringency conditions to nucleic acids that lack the reference exon but include one or more exons that are found adjacent to the reference exon the genome.

Given variable spacing of exons through eukaryotic genomes, the maximum length of single exon probes of the present invention is typically no more than 25 kb, often no more than 20 kb, 15 kb, 10 kb or 7.5 kb, or even no more than 5 kb, 4 kb, 3 kb, or even no more than about 2.5 kb in length.

The genome-derived single exon probes of the present invention can usefully include at least a first terminal priming sequence not found in contiguity with the rest of the probe sequence in the genome, and often will contain a second terminal priming sequence not found in contiguity with the rest of the probe sequence in the genome.

The present invention also provides isolated genome-derived nucleic acids that include nucleic acid sequence elements that control transcription of the GPCR-A-1 gene.

With a complete draft of the human genome now available, genomic sequences that are within the vicinity of the GPCR-A-1 coding region (and that are additional to those described with particularity herein) can readily be obtained by PCR amplification.

The isolated nucleic acids of the present invention can be composed of natural nucleotides in native 5′-3′ phosphodiester internucleoside linkage—e.g., DNA or RNA—or can contain any or all of nonnatural nucleotide analogues, normative internucleoside bonds, or post-synthesis modifications, either throughout the length of the nucleic acid or localized to one or more portions thereof.

As is well known in the art, when the isolated nucleic acid is used as a hybridization probe, the range of such nonnatural analogues, normative internucleoside bonds, or post-synthesis modifications will be limited to those that permit sequence-discriminating basepairing of the resulting nucleic acid. When used to direct expression or RNA or protein in vitro or in vivo, the range of such nonnatural analogues, normative internucleoside bonds, or post-synthesis modifications will be limited to those that permit the nucleic acid to function properly as a polymerization substrate. When the isolated nucleic acid is used as a therapeutic agent, the range of such changes will be limited to those that do not confer toxicity upon the isolated nucleic acid.

For example, when desired to be used as probes, the isolated nucleic acids of the present invention can usefully include nucleotide analogues that incorporate labels that are directly detectable, such as radiolabels or fluorophores, or nucleotide analogues that incorporate labels that can be visualized in a subsequent reaction, such as biotin or various haptens.

Common radiolabeled analogues include those labeled with ³³P, ³²P, and ³⁵S, such as α-³²P-dATP, α-³²P-dCTP, α-³²P-dGTP, α-³²P-dTTP, α-³²P-³²dATP, α-³²P-ATP, α-³²P-CTP, α-³²P-GTP, α-³²P-UTP, α-³⁵S-dATP, γ-³⁵S-GTP, γ-³³P-dATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Pharmacia Biotech, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY® TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA).

Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000), the disclosure of which is incorporated herein by reference in its entirety.

Haptens that are commonly conjugated to nucleotides for subsequent labeling include biotin (biotin-11-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-1-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

As another example, when desired to be used for antisense inhibition of transcription or translation, the isolated nucleic acids of the present invention can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties. Such altered internucloside bonds are often desired also when the isolated nucleic acid of the present invention is to be used for targeted gene correction, Gamper et al., Nucl. Acids Res. 28(21):4332-4339 (2000), the disclosures of which are incorporated herein by reference in its entirety.

Modified oligonucleotide backbones often preferred when the nucleic acid is to be used for antisense purposes are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Preferred modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,4.70,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

In other preferred oligonucleotide mimetics, both the sugar and the internucleoside linkage are replaced with novel groups, such as peptide nucleic acids (PNA).

In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages.

The uncharged nature of the PNA backbone provides PNA/DNA and PNA/RNA duplexes with a higher thermal stability than is found in DNA/DNA and DNA/RNA duplexes, resulting from the lack of charge repulsion between the PNA and DNA or RNA strand. In general, the Tm of a PNA/DNA or PNA/RNA duplex is 1C higher per base pair than the Tm of the corresponding DNA/DNA or DNA/RNA duplex (in 100 mM NaCl).

The neutral backbone also allows PNA to form stable DNA duplexes largely independent of salt concentration. At low ionic strength, PNA can be hybridized to a target sequence at temperatures that make DNA hybridization problematic or impossible. And unlike DNA/DNA duplex formation, PNA hybridization is possible in the absence of magnesium. Adjusting the ionic strength, therefore, is useful if competing DNA or RNA is present in the sample, or if the nucleic acid being probed contains a high level of secondary structure.

PNA also demonstrates greater specificity in binding to complementary DNA. A PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater.

Additionally, nucleases and proteases do not recognize the PNA polyamide backbone with nucleobase sidechains. As a result, PNA oligomers are resistant to degradation by enzymes, and the lifetime of these compounds is extended both in vivo and in vitro. In addition, PNA is stable over a wide pH range.

Because its backbone is formed from amide bonds, PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,331; and 5,719,262, each of which is herein incorporated by reference; automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.).

PNA chemistry and applications are reviewed, inter alia, in Ray et al., FASEB J. 14(9):1041-60 (2000); Nielsen et al., Pharmacol Toxicol. 86(1):3-7 (2000); Larsen et al., Biochim Biophys Acta. 1489(1):159-66 (1999); Nielsen, Curr. Opin. Struct. Biol. 9(3):353-7 (1999), and Nielsen, Curr. Opin. Biotechnol. 10(1):71-5 (1999), the disclosures of which are incorporated herein by reference in their entireties.

Differences from nucleic acid compositions found in nature—e.g., normative bases, altered internucleoside linkages, post-synthesis modification—can be present throughout the length of the nucleic acid or can, instead, usefully be localized to discrete portions thereof. As an example of the latter, chimeric nucleic acids can be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. As another example, chimeric nucleic acids comprising both DNA and PNA have been demonstrated to have utility in modified PCR reactions. See Misra et al., Biochem. 37: 1917-1925 (1998); see also Finn et al., Nucl. Acids Res. 24: 3357-3363 (1996), incorporated herein by reference.

Unless otherwise specified, nucleic acids of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Banér et al., Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994), the disclosures of which are incorporated herein by reference in their entireties. Triplex and quadruplex conformations, and their utilities, are reviewed in Praseuth et al., Biochim. Biophys. Acta. 1489(1):181-206 (1999); Fox, Curr. Med. Chem. 7(1):17-37 (2000); Kochetkova et al., Methods Mol. Biol. 130:189-201 (2000); Chan et al., J. Mol. Med. 75(4):267-82 (1997), the disclosures of which are incorporated herein by reference in their entireties.

The nucleic acids of the present invention can be detectably labeled.

Commonly-used labels include radionuclides, such as ³²P, ³³P, ³⁵S, ³H (and for NMR detection, ¹³C and ¹⁵N), haptens that can be detected by specific antibody or high affinity binding partner (such as avidin), and fluorophores.

As noted above, detectable labels can be incorporated by inclusion of labeled nucleotide analogues in the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach.

Analogues can also be incorporated during automated solid phase chemical synthesis.

As is well known, labels can also be incorporated after nucleic acid synthesis, with the 5′ phosphate and 3′ hydroxylproviding convenient sites for post-synthetic covalent attachment of detectable labels.

Various other post-synthetic approaches permit internal labeling of nucleic acids.

For example, fluorophores can be attached using a cisplatin reagent that reacts with the N7 of guanine residues (and, to a lesser extent, adenine bases) in DNA, RNA, and PNA to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer, Vol. 25, pp. 301-305 (1999); Jelsma et al., J. NIH Res. 5:82 (1994); Van Belkum et al., BioTechniques 16:148-153 (1994), incorporated herein by reference. As another example, nucleic acids can be labeled using a disulfide-containing linker (FastTag™ Reagent, Vector Laboratories, Inc., Burlingame, Calif., USA) that is photo- or thermally coupled to the target nucleic acid using aryl azide chemistry; after reduction, a free thiol is available for coupling to a hapten, fluorophore, sugar, affinity ligand, or other marker.

Multiple independent or interacting labels can be incorporated into the nucleic acids of the present invention.

For example, both a fluorophore and a moiety that in proximity thereto acts to quench fluorescence can be included to report specific hybridization through release of fluorescence quenching, Tyagi et al., Nature Biotechnol. 14: 303-308 (1996); Tyagi et al., Nature Biotechnol. 16, 49-53 (1998); Sokol et al., Proc. Natl. Acad. Sci. USA 95: 11538-11543 (1998); Kostrikis et al., Science 279:1228-1229 (1998); Marras et al., Genet. Anal. 14: 151-156 (1999); U.S. Pat. Nos. 5,846,726, 5,925,517, 5,925,517, or to report exonucleotidic excision, U.S. Pat. No. 5,538,848; Holland et al., Proc. Natl. Acad. Sci. USA 88:7276-7280 (1991); Heid et al., Genome Res. 6(10):986-94 (1996); Kuimelis et al., Nucleic Acids Symp Ser. (37):255-6 (1997); U.S. Pat. No. 5,723,591, the disclosures of which are incorporated herein by reference in their entireties.

So labeled, the isolated nucleic acids of the present invention can be used as probes, as further described below.

Nucleic acids of the present invention can also usefully be bound to a substrate. The substrate can porous or solid, planar or non-planar, unitary or distributed; the bond can be covalent or noncovalent. Bound to a substrate, nucleic acids of the present invention can be used as probes in their unlabeled state.

For example, the nucleic acids of the present invention can usefully be bound to a porous substrate, commonly a membrane, typically comprising nitrocellulose, nylon, or positively-charged derivatized nylon; so attached, the nucleic acids of the present invention can be used to detect GPCR-A-1 nucleic acids present within a labeled nucleic acid sample, either a sample of genomic nucleic acids or a sample of transcript-derived nucleic acids, e.g. by reverse dot blot.

The nucleic acids of the present invention can also usefully be bound to a solid substrate, such as glass, although other solid materials, such as amorphous silicon, crystalline silicon, or plastics, can also be used. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof.

Typically, the solid substrate will be rectangular, although other shapes, particularly disks and even spheres, present certain advantages. Particularly advantageous alternatives to glass slides as support substrates for array of nucleic acids are optical discs, as described in Demers, “Spatially Addressable Combinatorial Chemical Arrays in CD-ROM Format,” international patent publication WO 98/12559, incorporated herein by reference in its entirety.

The nucleic acids of the present invention can be attached covalently to a surface of the support substrate or applied to a derivatized surface in a chaotropic agent that facilitates denaturation and adherence by presumed noncovalent interactions, or some combination thereof.

The nucleic acids of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that include the nucleic acids of the present invention.

The isolated nucleic acids of the present invention can be used as hybridization probes to detect, characterize, and quantify GPCR-A-1 nucleic acids in, and isolate GPCR-A-1 nucleic acids from, both genomic and transcript-derived nucleic acid samples. When free in solution, such probes are typically, but not invariably, detectably labeled; bound to a substrate, as in a microarray, such probes are typically, but not invariably unlabeled.

For example, the isolated nucleic acids of the present invention can be used as probes to detect and characterize gross alterations in the GPCR-A-1 genomic locus, such as deletions, insertions, translocations, and duplications of the GPCR-A-1 genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acids of the present invention can be used as probes to isolate genomic clones that include the nucleic acids of the present invention, which thereafter can be restriction mapped and sequenced to identify deletions, insertions, translocations, and substitutions (single nucleotide polymorphisms, SNPs) at the sequence level.

The isolated nucleic acids of the present invention can also be used as probes to detect, characterize, and quantify GPCR-A-1 nucleic acids in, and isolate GPCR-A-1 nucleic acids from, transcript-derived nucleic acid samples.

For example, the isolated nucleic acids of the present invention can be used as hybridization probes to detect, characterize by length, and quantify GPCR-A-1 mRNA by northern blot of total or poly-A+—selected RNA samples. For example, the isolated nucleic acids of the present invention can be used as hybridization probes to detect, characterize by location, and quantify GPCR-A-1 message by in situ hybridization to tissue sections (see, e.g., Schwarchzacher et al., In Situ Hybridization, Springer-Verlag New York (2000) (ISBN: 0387915966), the disclosure of which is incorporated herein by reference in its entirety). For example, the isolated nucleic acids of the present invention can be used as hybridization probes to measure the representation of GPCR-A-1 clones in a cDNA library. For example, the isolated nucleic acids of the present invention can be used as hybridization probes to isolate GPCR-A-1 nucleic acids from cDNA libraries, permitting sequence level characterization of GPCR-A-1 messages, including identification of deletions, insertions, truncations—including deletions, insertions, and truncations of exons in alternatively spliced forms—and single nucleotide polymorphisms.

All of the aforementioned probe techniques are well within the skill in the art, and are described at greater length in standard texts such as Sambrook et al., Molecular Cloning: A Laboratory Manual (3^{rd} ed.), Cold Spring Harbor Laboratory Press (2001) (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X); and Walker et al. (eds.), The Nucleic Acids Protocols Handbook, Humana Press (2000) (ISBN: 0896034593), the disclosures of which are incorporated herein by reference in their entirety.

As described in the Examples herein below, the nucleic acids of the present invention can also be used to detect and quantify GPCR-A-1 nucleic acids in transcript-derived samples—that is, to measure expression of the GPCR-A-1 gene—when included in a microarray. Measurement of GPCR-A-1 expression has particular utility in both diagnosis and treatment of cancer, as further described in the Examples herein below.

As would be readily apparent to one of skill in the art, each GPCR-A-1 nucleic acid probe—whether labeled, substrate-bound, or both—is thus currently available for use as a tool for measuring the level of GPCR-A-1 expression in each of the tissues in which expression has already been confirmed, notably brain, heart, colon, testis and uterus, as well as kidney, bone marrow, skeletal muscle and placenta. The utility is specific to the probe: under high stringency conditions, the probe reports the level of expression of message specifically containing that portion of the GPCR-A-1 gene included within the probe.

Measuring tools are well known in many arts, not just in molecular biology, and are known to possess credible, specific, and substantial utility. For example, U.S. Pat. No. 6,016,191 describes and claims a tool for measuring characteristics of fluid flow in a hydrocarbon well; U.S. Pat. No. 6,042,549 describes and claims a device for measuring exercise intensity; U.S. Pat. No. 5,889,351 describes and claims a device for measuring viscosity and for measuring characteristics of a fluid; U.S. Pat. No. 5,570,694 describes and claims a device for measuring blood pressure; U.S. Pat. No. 5,930,143 describes and claims a device for measuring the dimensions of machine tools; U.S. Pat. No. 5,279,044 describes and claims a measuring device for determining an absolute position of a movable element; U.S. Pat. No. 5,186,042 describes and claims a device for measuring action force of a wheel; and U.S. Pat. No. 4,246,774 describes and claims a device for measuring the draft of smoking articles such as cigarettes.

As for tissues not yet demonstrated to express GPCR-A-1, the GPCR-A-1 nucleic acid probes of the present invention are currently available as tools for surveying such tissues to detect the presence of GPCR-A-1 nucleic acids.

Survey tools—i.e., tools for determining the presence and/or location of a desired object by search of an area—are well known in many arts, not just in molecular biology, and are known to possess credible, specific, and substantial utility. For example, U.S. Pat. No. 6,046,800 describes and claims a device for surveying an area for objects that move; U.S. Pat. No. 6,025,201 describes and claims an apparatus for locating and discriminating platelets from non-platelet particles or cells on a cell-by-cell basis in a whole blood sample; U.S. Pat. No. 5,990,689 describes and claims a device for detecting and locating anomalies in the electromagnetic protection of a system; U.S. Pat. No. 5,984,175 describes and claims a device for detecting and identifying wearable user identification units; U.S. Pat. No. 3,980,986 (“Oil well survey tool”), describes and claims a tool for finding the position of a drill bit working at the bottom of a borehole.

As noted above, the nucleic acid probes of the present invention are useful in constructing microarrays; the microarrays, in turn, are products of manufacture that are useful for measuring and for surveying gene expression.

When included on a microarray, each GPCR-A-1 nucleic acid probe makes the microarray specifically useful for detecting that portion of the GPCR-A-1 gene included within the probe, thus imparting upon the microarray device the ability to detect a signal where, absent such probe, it would have reported no signal. This utility makes each individual probe on such microarray akin to an antenna, circuit, firmware or software element included in an electronic apparatus, where the antenna, circuit, firmware or software element imparts upon the apparatus the ability newly and additionally to detect signal in a portion of the radio-frequency spectrum where previously it could not; such devices are known to have specific, substantial, and credible utility.

Changes in the level of expression need not be observed for the measurement of expression to have utility.

For example, where gene expression analysis is used to assess toxicity of chemical agents on cells, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part.

WO 99/58720, incorporated herein by reference in its entirety, provides methods for quantifying the relatedness of a first and second gene expression profile and for ordering the relatedness of a plurality of gene expression profiles, without regard to the identity or function of the genes whose expression is used in the calculation.

Gene expression analysis, including gene expression analysis by microarray hybridization, is, of course, principally a laboratory-based art. Devices and apparatus used principally in laboratories to facilitate laboratory research are well-established to possess specific, substantial, and credible utility. For example, U.S. Pat. No. 6,001,233 describes and claims a gel electrophoresis apparatus having a cam-activated clamp; for example, U.S. Pat. No. 6,051,831 describes and claims a high mass detector for use in time-of-flight mass spectrometers; for example, U.S. Pat. No. 5,824,269 describes and claims a flow cytometer—as is well known, few gel electrophoresis apparatuses, TOF-MS devices, or flow cytometers are sold for consumer use.

Indeed, and in particular, nucleic acid microarrays, as devices intended for laboratory use in measuring gene expression, are well-established to have specific, substantial and credible utility. Thus, the microarrays of the present invention have at least the specific, substantial and credible utilities of the microarrays claimed as devices and articles of manufacture in the following U.S. patents, the disclosures of each of which is incorporated herein by reference: U.S. Pat. No. 5,445,934 (“Array of oligonucleotides on a solid substrate”); U.S. Pat. No. 5,744,305 (“Arrays of materials attached to a substrate”); and U.S. Pat. No. 6,004,752 (“Solid support with attached molecules”).

Genome-derived single exon probes and genome-derived single exon probe microarrays have the additional utility, inter alia, of permitting high-throughput detection of splice variants of the nucleic acids of the present invention, as further described in copending and commonly owned U.S. patent application Ser. No. 09/632,366, filed Aug. 3, 2000, the disclosure of which is incorporated herein by reference in its entirety.

The isolated nucleic acids of the present invention can also be used to prime synthesis of nucleic acid, for purpose of either analysis or isolation, using mRNA, cDNA, or genomic DNA as template.

For use as primers, at least 17 contiguous nucleotides of the isolated nucleic acids of the present invention will be used. Often, at least 18, 19, or 20 contiguous nucleotides of the nucleic acids of the present invention will be used, and on occasion at least 20, 22, 24, or 25 contiguous nucleotides of the nucleic acids of the present invention will be used, and even 30 nucleotides or more of the nucleic acids of the present invention can be used to prime specific synthesis.

The nucleic acid primers of the present invention can be used, for example, to prime first strand cDNA synthesis on an mRNA template.

Such primer extension can be done directly to analyze the message. Alternatively, synthesis on an mRNA template can be done to produce first strand cDNA. The first strand cDNA can thereafter be used, inter alia, directly as a single-stranded probe, as above-described, as a template for sequencing—permitting identification of alterations, including deletions, insertions, and substitutions, both normal allelic variants and mutations associated with abnormal phenotypes- or as a template, either for second strand cDNA synthesis (e.g., as an antecedent to insertion into a cloning or expression vector), or for amplification.

The nucleic acid primers of the present invention can also be used, for example, to prime single base extension (SBE) for SNP detection (see, e.g., U.S. Pat. No. 6,004,744, the disclosure of which is incorporated herein by reference in its entirety).

As another example, the nucleic acid primers of the present invention can be used to prime amplification of GPCR-A-1 nucleic acids, using transcript-derived or genomic DNA as template.

Primer-directed amplification methods are now well-established in the art. Methods for performing the polymerase chain reaction (PCR) are compiled, inter alia, in McPherson, PCR (Basics: From Background to Bench), Springer Verlag (2000) (ISBN: 0387916008); Innis et al. (eds.), PCR Applications: Protocols for Functional Genomics, Academic Press (1999) (ISBN: 0123721857); Gelfand et al. (eds.), PCR Strategies, Academic Press (1998) (ISBN: 0123721822); Newton et al., PCR, Springer-Verlag New York (1997) (ISBN: 0387915060); Burke (ed.), PCR: Essential Techniques, John Wiley & Son Ltd (1996) (ISBN: 047195697X); White (ed.), PCR Cloning Protocols: From Molecular Cloning to Genetic Engineering, Vol. 67, Humana Press (1996) (ISBN: 0896033430); McPherson et al. (eds.), PCR 2: A Practical Approach, Oxford University Press, Inc. (1995) (ISBN: 0199634254), the disclosures of which are incorporated herein by reference in their entireties. Methods for performing RT-PCR are collected, e.g., in Siebert et al. (eds.), Gene Cloning and Analysis by RT-PCR, Eaton Publishing Company/Bio Techniques Books Division, 1998 (ISBN: 1881299147); Siebert (ed.), PCR Technique:RT-PCR, Eaton Publishing Company/BioTechniques Books (1995) (ISBN:1881299139), the disclosure of which is incorporated herein by reference in its entirety.

Isothermal amplification approaches, such as rolling circle amplification, are also now well-described. See, e.g., Schweitzer et al., Curr. Opin. Biotechnol. 12(1):21-7 (2001); U.S. Pat. Nos. 6,235,502, 6,221,603, 6,210,884, 6,183,960, 5,854,033, 5,714,320, 5,648,245, and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

As further described below, nucleic acids of the present invention, inserted into vectors that flank the nucleic acid insert with a phage promoter, such as T7, T3, or SP6 promoter, can be used to drive in vitro expression of RNA complementary to either strand of the nucleic acid of the present invention. The RNA can be used, inter alia, as a single-stranded probe, in cDNA-mRNA subtraction, or for in vitro translation.

As will be further discussed herein below, nucleic acids of the present invention that encode GPCR-A-1 protein or portions thereof can be used, inter alia, to express the GPCR-A-1 proteins or protein fragments, either alone, or as part of fusion proteins.

Expression can be from genomic nucleic acids of the present invention, or from transcript-derived nucleic acids of the present invention.

Where protein expression is effected from genomic DNA, expression will typically be effected in eukaryotic, typically mammalian, cells capable of splicing introns from the initial RNA transcript. Expression can be driven from episomal vectors, such as EBV-based vectors, or can be effected from genomic DNA integrated into a host cell chromosome. As will be more fully described below, where expression is from transcript-derived (or otherwise intron-less) nucleic acids of the present invention, expression can be effected in wide variety of prokaryotic or eukaryotic cells.

Expressed in vitro, the protein, protein fragment, or protein fusion can thereafter be isolated, to be used, inter alia, as a standard in immunoassays specific for the proteins, or protein isoforms, of the present invention; to be used as a therapeutic agent, e.g., to be administered as passive replacement therapy in individuals deficient in the proteins of the present invention, or to be administered as a vaccine; to be used for in vitro production of specific antibody, the antibody thereafter to be used, e.g., as an analytical reagent for detection and quantitation of the proteins of the present invention or to be used as an immunotherapeutic agent.

The isolated nucleic acids of the present invention can also be used to drive in vivo expression of the proteins of the present invention. In vivo expression can be driven from a vector—typically a viral vector, often a vector based upon a replication incompetent retrovirus, an adenovirus, or an adeno-associated virus (AAV)—for purpose of gene therapy. In vivo expression can also be driven from signals endogenous to the nucleic acid or from a vector, often a plasmid vector, such as pVAX1 (Invitrogen, Carlsbad Calif., USA), for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; 6,204,250, the disclosures of which are incorporated herein by reference in their entireties.

The nucleic acids of the present invention can also be used for antisense inhibition of transcription or translation. See Phillips (ed.), Antisense Technology, Part B, Methods in Enzymology Vol. 314, Academic Press, Inc. (1999) (ISBN: 012182215X); Phillips (ed.), Antisense Technology, Part A, Methods in Enzymology Vol. 313, Academic Press, Inc. (1999) (ISBN: 0121822141); Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Agrawal et al. (eds.), Antisense Research and Application, Springer-Verlag New York, Inc. (1998) (ISBN: 3540638334); Lichtenstein et al. (eds.), Antisense Technology: A Practical Approach, Vol. 185, Oxford University Press; INC. (1998) (ISBN: 0199635838); Gibson (ed.), Antisense and Ribozyme Methodology: Laboratory Companion, Chapman & Hall (1997) (ISBN: 3826100794); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties.

Nucleic acids of the present invention, particularly cDNAs of the present invention, that encode full-length human GPCR-A-1 protein isoforms, have additional, well-recognized, immediate, real world utility as commercial products of manufacture suitable for sale.

For example, Invitrogen Corp. (Carlsbad, Calif., USA), through its Research Genetics subsidiary, sells full length human cDNAs cloned into one of a selection of expression vectors as GeneStorm® expression-ready clones; utility is specific for the gene, since each gene is capable of being ordered separately and has a distinct catalogue number, and utility is substantial, each clone selling for $650.00 US. Similarly, Incyte Genomics (Palo Alto, Calif., USA) sells clones from public and proprietary sources in multi-well plates or individual tubes.

Nucleic acids of the present invention that include genomic regions encoding the human GPCR-A-1 protein, or portions thereof, have yet further utilities.

For example, genomic nucleic acids of the present invention can be used as amplification substrates, e.g. for preparation of genome-derived single exon probes of the present invention, as described above and in copending and commonly-owned U.S. patent application Ser. No. 09/864,761, filed May 23, 2001, Ser. No. 09/774,203, filed Jan. 29, 2001, and Ser. No. 09/632,366, filed Aug. 3, 2000, the disclosures of which are incorporated herein by reference in their entireties.

As another example, genomic nucleic acids of the present invention can be integrated non-homologously into the genome of somatic cells, e.g. CHO cells, COS cells, or 293 cells, with or without amplification of the insertional locus, in order, e.g., to create stable cell lines capable of producing the proteins of the present invention.

As another example, more fully described herein below, genomic nucleic acids of the present invention can be integrated nonhomologously into embryonic stem (ES) cells to create transgenic non-human animals capable of producing the proteins of the present invention.

Genomic nucleic acids of the present invention can also be used to target homologous recombination to the human GPCR-A-1 locus. See, e.g., U.S. Pat. Nos. 6,187,305; 6,204,061; 5,631,153; 5,627,059; 5,487,992; 5,464,764; 5,614,396; 5,527,695 and 6,063,630; and Kmiec et al. (eds.), Gene Targeting Protocols, Vol. 133, Humana Press (2000) (ISBN: 0896033600); Joyner (ed.), Gene Targeting: A Practical Approach, Oxford University Press, Inc. (2000) (ISBN: 0199637938); Sedivy et al., Gene Targeting, Oxford University Press (1998) (ISBN: 071677013X); Tymms et al. (eds.), Gene Knockout Protocols, Humana Press (2000) (ISBN: 0896035727); Mak et al. (eds.), The Gene Knockout FactsBook, Vol. 2, Academic Press, Inc. (1998) (ISBN: 0124660444); Torres et al., Laboratory Protocols for Conditional Gene Targeting, Oxford University Press (1997) (ISBN: 019963677X); Vega (ed.), Gene Targeting, CRC Press, LLC (1994) (ISBN: 084938950X), the disclosures of which are incorporated herein by reference in their entireties.

Where the genomic region includes transcription regulatory elements, homologous recombination can be used to alter the expression of GPCR-A-1, both for purpose of in vitro production of GPCR-A-1 protein from human cells, and for purpose of gene therapy. See, e.g., U.S. Pat. Nos. 5,981,214, 6,048,524; 5,272,071.

Fragments of the nucleic acids of the present invention smaller than those typically used for homologous recombination can also be used for targeted gene correction or alteration, possibly by cellular mechanisms different from those engaged during homologous recombination.

For example, partially duplexed RNA/DNA chimeras have been shown to have utility in targeted gene correction, U.S. Pat. Nos. 5,945,339, 5,888,983, 5,871,984, 5,795,972, 5,780,296, 5,760,012, 5,756,325, 5,731,181, the disclosures of which are incorporated herein by reference in their entireties. So too have small oligonucleotides fused to triplexing domains have been shown to have utility in targeted gene correction, Culver et al., “Correction of chromosomal point mutations in human cells with bifunctional oligonucleotides,” Nature Biotechnol. 17(10):989-93 (1999), as have oligonucleotides having modified terminal bases or modified terminal internucleoside bonds, Gamper et al., Nucl. Acids Res. 28(21):4332-9 (2000), the disclosures of which are incorporated herein by reference.

The isolated nucleic acids of the present invention can also be used to provide the initial substrate for recombinant engineering of GPCR-A-1 protein variants having desired phenotypic improvements. Such engineering includes, for example, site-directed mutagenesis, random mutagenesis with subsequent functional screening, and more elegant schemes for recombinant evolution of proteins, as are described, inter alia, in U.S. Pat. Nos. 6,180,406; 6,165,793; 6,117,679; and 6,096,548, the disclosures of which are incorporated herein by reference in their entireties.

Nucleic acids of the present invention can be obtained by using the labeled probes of the present invention to probe nucleic acid samples, such as genomic libraries, cDNA libraries, and mRNA samples, by standard techniques. Nucleic acids of the present invention can also be obtained by amplification, using the nucleic acid primers of the present invention, as further demonstrated in Example 1, herein below. Nucleic acids of the present invention of fewer than about 100 nt can also be synthesized chemically, typically by solid phase synthesis using commercially available automated synthesizers.

“Full Length” GPCR-A-1 Nucleic Acids

In a first series of nucleic acid embodiments, the invention provides isolated nucleic acids that encode the entirety of the GPCR-A-1 protein. As discussed above, the “full-length” nucleic acids of the present invention can be used, inter alia, to express full length GPCR-A-1 protein. The full-length nucleic acids can also be used as nucleic acid probes; used as probes, the isolated nucleic acids of these embodiments will hybridize to GPCR-A-1.

In a first such embodiment, the invention provides an isolated nucleic acid comprising (i) the nucleotide sequence of SEQ ID NO: 1 or (ii) the complement of (i). SEQ ID NO: 1 presents, the entire cDNA of GPCR-A-1, including the 5′ untranslated (UT) region and 3′ UT.

In a second embodiment, the invention provides an isolated nucleic acid comprising (i) the nucleotide sequence of SEQ ID NO: 2 (ii) a degenerate variant of the nucleotide sequence of SEQ ID NO: 2 or (iii) the complement of (i) or (ii). SEQ ID NO: 2 presents the open reading frame (ORF) from SEQ ID NO: 1

In a third embodiment, the invention provides an isolated nucleic acid comprising (i) a nucleotide sequence that encodes a polypeptide with the amino acid sequence of SEQ ID NO: 3 or (ii) the complement of a nucleotide sequence that encodes a polypeptide with the amino acid sequence of SEQ ID NO: 3. SEQ ID NO: 3 provides the amino acid sequence of GPCR-A-1.

In a fourth embodiment, the invention provides an isolated nucleic acid having a nucleotide sequence that (i) encodes a polypeptide having the sequence of SEQ ID NO: 3, (ii) encodes a polypeptide having the sequence of SEQ ID NO: 3 with conservative amino acid substitutions, or (iii) that is the complement of (i) or (ii), where SEQ ID NO: 3 provides the amino acid sequence of GPCR-A-1.

Selected Partial Nucleic Acids

In a second series of nucleic acid embodiments, the invention provides isolated nucleic acids that encode select portions of GPCR-A-1. As will be further discussed herein below, these “partial” nucleic acids can be used, inter alia, to express specific portions of the GPCR-A-1. These “partial” nucleic acids can also be used, inter alia, as nucleic probes.

In a first such embodiment, the invention provides an isolated nucleic acid comprising (i) the nucleotide sequence of SEQ ID NO: 4, (ii) a degenerate variant of SEQ ID NO: 4, or (iii) the complement of (i) or (ii), wherein the isolated nucleic acid is no more than about 100 kb in length, typically no more than about 75 kb in length, more typically no more than about 50 kb length. SEQ ID NO: 4 encodes a novel portion of GPCR-A-1. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) the nucleotide sequence of SEQ ID NO: 5 (ii) a degenerate variant of SEQ ID NO: 5 or (iii) the complement of (i) or (ii), wherein the isolated nucleic acid is no more than about 100 kb in length, typically no more than about 75 kb in length, more typically no more than about 50 kb length. SEQ ID NO: 5 encodes another novel portion of GPCR-A-1. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 6 or (ii) the complement of a nucleotide sequence that encodes SEQ ID NO: 6, wherein the isolated nucleic acid is no more than about 100 kb in length, typically no more than about 75 kb in length, frequently no more than about 50 kb in length. SEQ ID NO: 6 is the amino acid sequence encoded by the portion of GPCR-A-1 (SEQ ID NO; 5) not found in any EST fragments. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) a nucleotide sequence that encodes SEQ ID NO: 6, (ii) a nucleotide sequence that encodes SEQ ID NO: 6 with conservative substitutions, or (iii) the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In another embodiment, the invention provides an isolated nucleic acid comprising (i) the nucleotide sequence of SEQ ID NO: 7 (ii) a degenerate variant of SEQ ID NO: 7 or (iii) the complement of (i) or (ii), wherein the isolated nucleic acid is no more than about 100 kb in length, typically no more than about 75 kb in length, more typically no more than about 50 kb length. SEQ ID NO: 7 encodes another novel portion of GPCR-A-1. Often, the isolated nucleic acids of this embodiment are no more than about 25 kb in length, often no more than about 15 kb in length, and frequently no more than about 10 kb in length.

Cross-Hybridizing Nucleic Acids

In another series of nucleic acid embodiments, the invention provides isolated nucleic acids that hybridize to various of the GPCR-A-1 nucleic acids of the present invention. These cross-hybridizing nucleic acids can be used, inter alia, as probes for, and to drive expression of, proteins that are related to GPCR-A-1 of the present invention as further isoforms, homologues, paralogues, or orthologues.

In a first such embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 4 or the complement of SEQ ID NO: 4, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 5 or the complement of SEQ ID NO: 5, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 5 or the complement of SEQ ID NO: 5, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under high stringency conditions to a hybridization probe the nucleotide sequence of which (i) encodes a polypeptide having the sequence of SEQ ID NO: 6, (ii) encodes a polypeptide having the sequence of SEQ ID NO: 6 with conservative amino acid substitutions, or (iii) is the complement of (i) or (ii), wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a first such embodiment, the invention provides an isolated nucleic acid comprising a sequence that hybridizes under high stringency conditions to a probe the nucleotide sequence of which consists of at least 17 nt, 18, 19, 20, 21, 22, 23, 24, 25, 30, 40, or 50 nt of SEQ ID NO: 7 or the complement of SEQ ID NO: 7, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

In a further embodiment, the invention provides an isolated nucleic acid comprising a sequence that hybridizes under moderate stringency conditions to a probe the nucleotide sequence of which consists of at least 17 nt, 18, 19, 20, 21, 22, 23, 24, 25, 30, 40, or 50 nt of SEQ ID NO: 7 or the complement of SEQ ID NO: 7, wherein the isolated nucleic acid is no more than about 100 kb in length, typically no more than about 75 kb in length, and often no more than about 50 kb in length. Often, the isolated nucleic acids of this embodiment are no more than about 25 kb in length, often no more than about 15 kb in length, and frequently no more than about 10 kb in length.

Particularly Useful Nucleic Acids

Particularly useful among the above-described nucleic acids are those that are expressed, or the complement of which are expressed, in brain, heart, colon, testis and uterus, as well as in kidney, bone marrow, skeletal muscle and placenta, preferably at a level greater than that in liver or lung, typically at a level at least two-fold that in liver or lung, often at least three-fold, four-fold, or even five-fold that in liver or lung.

Also particularly useful among the above-described nucleic acids are those that encode, or the complement of which encode a G protein coupled receptor that functions as a tumor suppressor, mutations of which cause a variety of cancers.

Other particularly useful embodiments of the nucleic acids above-described are those that encode, or the complement of which encode, a polypeptide having at least one DUF6 motif, and especially those that encode two DUF6 motifs, those that encode one transmembrane motif, and especially those that encode seven transmembrane motifs

Nucleic Acid Fragments

In another series of nucleic acid embodiments, the invention provides fragments of various of the isolated nucleic acids of the present invention which prove useful, inter alia, as nucleic acid probes, as amplification primers, and to direct expression or synthesis of epitopic or immunogenic protein fragments.

In a first such embodiment, the invention provides an isolated nucleic acid comprising at least 17 nucleotides, 18 nucleotides, 20 nucleotides, 24 nucleotides, or 25 nucleotides of (i) SEQ ID NO: 4, (ii) a degenerate variant of SEQ ID NO: 4, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising at least 17 nucleotides, 18 nucleotides, 20 nucleotides, 24 nucleotides, or 25 nucleotides of (i) SEQ ID NO: 5, (ii) a degenerate variant of SEQ ID NO: 5, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising (i) a nucleotide sequence that encodes a peptide of at least 8 contiguous amino acids of SEQ ID NO: 6, (ii) a nucleotide sequence that encodes a peptide of at least 15 contiguous amino acids of SEQ ID NO: 6, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

The invention also provides an isolated nucleic acid comprising a nucleotide sequence that encodes (i) a polypeptide having the sequence of at least 8 contiguous amino acids of SEQ ID NO: 6 with conservative amino acid substitutions, (ii) a polypeptide having the sequence of at least 15 contiguous amino acids of SEQ ID NO: 6 with conservative amino acid substitutions, (iii) a polypeptide having the sequence of at least 8 contiguous amino acids of SEQ ID NO: 6 with moderately conservative substitutions, (iv) a polypeptide having the sequence of at least 15 contiguous amino acids of SEQ ID NO: 6 with moderately conservative substitutions, or (v) the complement of any of (i)-(iv), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another such embodiment, the invention provides an isolated nucleic acid comprising at least 17 nucleotides, 18 nucleotides, 20 nucleotides, 24 nucleotides, or 25 nucleotides of (i) SEQ ID NO: 7, (ii) a degenerate variant of SEQ ID NO: 7, or (iii) the complement of (i) or (ii), wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

Single Exon Probes

The invention further provides genome-derived single exon probes having portions of no more than one exon of the GPCR-A-1 gene. As further described in commonly owned and copending U.S. patent application Ser. No. 09/632,366, filed Aug. 3, 2000 (“Methods and Apparatus for High Throughput Detection and Characterization of alternatively Spliced Genes”), the disclosure of which is incorporated herein by reference in its entirety, such single exon probes have particular utility in identifying and characterizing splice variants. In particular, such single exon probes are useful for identifying and discriminating the expression of distinct isoforms of GPCR-A-1.

In a first embodiment, the invention provides an isolated nucleic acid comprising a nucleotide sequence of no more than one portion of SEQ ID NOs: 8-15 or the complement of SEQ ID NOs: 8-15, wherein the portion comprises at least 17 contiguous nucleotides, 18 contiguous nucleotides, 20 contiguous nucleotides, 24 contiguous nucleotides, 25 contiguous nucleotides, or 50 contiguous nucleotides of any one of SEQ ID NOs: 8-15, or their complement. In a further embodiment, the exonic portion comprises the entirety of the referenced SEQ ID NO: or its complement.

In other embodiments, the invention provides isolated single exon probes having the nucleotide sequence of any one of SEQ ID NOs: 16-23.

Transcription Control Nucleic Acids

In another aspect, the present invention provides genome-derived isolated nucleic acids that include nucleic acid sequence elements that control transcription of the GPCR-A-1 gene. These nucleic acids can be used, inter alia, to drive expression of heterologous coding regions in recombinant constructs, thus conferring upon such heterologous coding regions the expression pattern of the native GPCR-A-1 gene. These nucleic acids can also be used, conversely, to target heterologous transcription control elements to the GPCR-A-1 genomic locus, altering the expression pattern of the GPCR-A-1 gene itself.

In a first such embodiment, the invention provides an isolated nucleic acid comprising the nucleotide sequence of SEQ ID NO: 24 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

In another embodiment, the invention provides an isolated nucleic acid comprising at least 17, 18, 20, 24, or 25 nucleotides of the sequence of SEQ ID NO: 24 or its complement, wherein the isolated nucleic acid is no more than about 100 kb in length, typically no more than about 75 kb in length, more typically no more than about 50 kb in length. Often, the isolated nucleic acids of this embodiment are no more than about 25 kb in length, often no more than about 15 kb in length, and frequently no more than about 10 kb in length.

Vectors and Host Cells

In another aspect, the present invention provides vectors that comprise one or more of the isolated nucleic acids of the present invention, and host cells in which such vectors have been introduced.

The vectors can be used, inter alia, for propagating the nucleic acids of the present invention in host cells (cloning vectors), for shuttling the nucleic acids of the present invention between host cells derived from disparate organisms (shuttle vectors), for inserting the nucleic acids of the present invention into host cell chromosomes (insertion vectors), for expressing sense or antisense RNA transcripts of the nucleic acids of the present invention in vitro or within a host cell, and for expressing polypeptides encoded by the nucleic acids of the present invention, alone or as fusions to heterologous polypeptides. Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems: Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al. (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. Furthermore, an enormous variety of vectors are available commercially. Use of existing vectors and modifications thereof being well within the skill in the art, only basic features need be described here.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the present invention will further include at least one nucleic acid of the present invention inserted into the vector in at least one location.

Where present, the origin of replication and selectable markers are chosen based upon the desired host cell or host cells; the host cells, in turn, are selected based upon the desired application.

For example, prokaryotic cells, typically E. coli, are typically chosen for cloning. In such case, vector replication is predicated on the replication strategies of coliform-infecting phage—such as phage lambda, M13, T7, T3 and P1- or on the replication origin of autonomously replicating episomes, notably the ColE1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin, zeocin; auxotrophic markers can also be used.

As another example, yeast cells, typically S. cerevisiae, are chosen, inter alia, for eukaryotic genetic studies, due to the ease of targeting genetic changes by homologous recombination and to the ready ability to complement genetic defects using recombinantly expressed proteins, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast.

Integrative YIp vectors do not replicate autonomously, but integrate, typically in single copy, into the yeast genome at low frequencies and thus replicate as part of the host cell chromosome; these vectors lack an origin of replication that is functional in yeast, although they typically have at least one origin of replication suitable for propagation of the vector in bacterial cells. YEp vectors, in contrast, replicate episomally and autonomously due to presence of the yeast 2 micron plasmid origin (2 μm ori). The YCp yeast centromere plasmid vectors are autonomously replicating vectors containing centromere sequences, CEN, and autonomously replicating sequences, ARS; the ARS sequences are believed to correspond to the natural replication origins of yeast chromosomes. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trp1-D1 and lys2-201. The URA3 and LYS2 yeast genes further permit negative selection based on specific inhibitors, 5-fluoro-orotic acid (FOA) and α-aminoadipic acid (aAA), respectively, that prevent growth of the prototrophic strains but allows growth of the ura3 and lys2 mutants, respectively. Other selectable markers confer resistance to, e.g., zeocin.

As yet another example, insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

As yet another example, mammalian cells are often chosen for expression of proteins intended as pharmaceutical agents, and are also chosen as host cells for screening of potential agonist and antagonists of a protein or a physiological pathway.

Where mammalian cells are chosen as host cells, vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Plant cells can also be used for expression, with the vector replicon typically derived from a plant virus (e.g., cauliflower mosaic virus, CaMV; tobacco mosaic virus, TMV) and selectable markers chosen for suitability in plants.

For propagation of nucleic acids of the present invention that are larger than can readily be accomodated in vectors derived from plasmids or virus, the invention further provides artificial chromosomes—BACs, YACs, PACs, and HACs—that comprise GPCR-A-1 nucleic acids, often genomic nucleic acids.

The BAC system is based on the well-characterized E. coli F-factor, a low copy plasmid that exists in a supercoiled circular form in host cells. The structural features of the F-factor allow stable maintenance of individual human DNA clones as well as easy manipulation of the cloned DNA. See Shizuya et al., Keio J. Med. 50(1):26-30 (2001); Shizuya et al., Proc. Natl. Acad. Sci. USA 89(18):8794-7 (1992).

YACs are based on yeast linear plasmids, denoted YLp, containing homologous or heterologous DNA sequences that function as telomeres (TEL) in vivo, as well as containing yeast ARS (origins of replication) and CEN (centromeres) segments.

HACs are human artifical chromosomes. Kuroiwa et al., Nature Biotechnol. 18(10):1086-90 (2000); Henning et al., Proc. Natl. Acad. Sci. USA 96(2):592-7 (1999); Harrington et al., Nature Genet. 15(4):345-55 (1997). In one version, long synthetic arrays of alpha satellite DNA are combined with telomeric DNA and genomic DNA to generate linear microchromosomes that are mitotically and cytogenetically stable in the absence of selection.

PACs are P1-derived artificial chromosomes. Sternberg, Proc. Natl. Acad. Sci. USA 87(1):103-7 (1990); Sternberg et al., New Biol. 2 (2):151-62 (1990); Pierce et al., Proc. Natl. Acad. Sci. USA 89(6):2056-60 (1992).

Vectors of the present invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Expression vectors of the present invention—that is, those vectors that will drive expression of polypeptides from the inserted heterologous nucleic acid—will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences.

For example, vectors for expressing proteins of the present invention in prokaryotic cells, typically E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), or the araBAD operon. Often, such prokaryotic expression vectors will further include transcription terminators, such as the aspA terminator, and elements that facilitate translation, such as a consensus ribosome binding site and translation termination codon, Schomer et al., Proc. Natl. Acad. Sci. USA 83:8506-8510 (1986).

As another example, vectors for expressing proteins of the present invention in yeast cells, typically S. cerevisiae, will include a yeast promoter, such as the CYC1 promoter, the GAL1 promoter, ADH1 promoter, or the GPD promoter, and will typically have elements that facilitate transcription termination, such as the transcription termination signals from the CYC1 or ADH1 gene.

As another example, vectors for expressing proteins of the present invention in mammalian cells will include a promoter active in mammalian cells. Such promoters are often drawn from mammalian viruses—such as the enhancer-promoter sequences from the immediate early gene of the human cytomegalovirus (CMV), the enhancer-promoter sequences from the Rous sarcoma virus long terminal repeat (RSV LTR), and the enhancer-promoter from SV40. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Vector-drive protein expression can be constitutive or inducible.

Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline.

As another example of inducible elements, hormone response elements, such as the glucocorticoid response element (GRE) and the estrogen response element (ERE), can confer hormone inducibility where vectors are used for expression in cells having the respective hormone receptors. To reduce background levels of expression, elements responsive to ecdysone, an insect hormone, can be used instead, with coexpression of the ecdysone receptor.

Expression vectors can be designed to fuse the expressed polypeptide to small protein tags that facilitate purification and/or visualization.

For example, proteins of the present invention can be expressed with a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). As another example, the fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the proteins of the present invention can be expressed as a fusion to glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione.

Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG® antibody (Stratagene, La Jolla, Calif., USA), and the HA epitope.

For secretion of expressed proteins, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Vectors for phage display fuse the encoded polypeptide to, e.g., the gene III protein (pIII) or gene VIII protein (pVIII) for display on the surface of filamentous phage, such as M13. See Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, San Diego: Academic Press, Inc., 1996;
Abelson et al. (eds.), Combinatorial Chemistry, Methods in Enzymology vol. 267, Academic Press (May 1996).

Vectors for yeast display, e.g. the PYDI yeast display vector (Invitrogen, Carlsbad, Calif., USA), use the a-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea victoria (“GFP”) and its variants. These proteins are intrinsically fluorescent: the GFP-like chromophore is entirely encoded by its amino acid sequence and can fluoresce without requirement for cofactor or substrate.

Structurally, the GFP-like chromophore comprises an 11-stranded β-barrel (β-can) with a central α-helix, the central α-helix having a conjugated π-resonance system that includes two aromatic ring systems and the bridge between them. The π-resonance system is created by autocatalytic cyclization among amino acids; cyclization proceeds through an imidazolinone intermediate, with subsequent dehydrogenation by molecular oxygen at the Cα-Cβ bond of a participating tyrosine.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., “Deletions of the Aequorea victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety. A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormo et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in the fusion proteins of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein, Methods in Enzymol. Vol. 302, pp 378-394 (1999), incorporated herein by reference in its entirety. A variety of such modified chromophores are now commercially available and can readily be used in the fusion proteins of the present invention.

Fusions to the IgG Fc region increase serum half life of protein pharmaceutical products through interaction with the FcRn receptor (also denominated the FcRp receptor and the Brambell receptor, FcRb), further described in international patent application nos. WO 97/43316, WO 97/34631, WO 96/32478, WO 96/18412.

For long-term, high-yield recombinant production of the proteins, protein fusions, and protein fragments of the present invention, stable expression is particularly useful.

Stable expression is readily achieved by integration into the host cell genome of vectors having selectable markers, followed by selection for integrants.

For example, the pUB6/V5-His A, B, and C vectors (Invitrogen, Carlsbad, Calif., USA) are designed for high-level stable expression of heterologous proteins in a wide range of mammalian tissue types and cell lines. pUB6/V5-His uses the promoter/enhancer sequence from the human ubiquitin C gene to drive expression of recombinant proteins: expression levels in 293, CHO, and NIH3T3 cells are comparable to levels from the CMV and human EF-1a promoters. The bsd gene permits rapid selection of stably transfected mammalian cells with the potent antibiotic blasticidin.

Replication incompetent retroviral vectors, typically derived from Moloney murine leukemia virus, prove particularly useful for creating stable transfectants having integrated provirus. The highly efficient transduction machinery of retroviruses, coupled with the availability of a variety of packaging cell lines—such as RetroPack™ PT 67, EcoPack2™-293, AmphoPack-293, GP2-293 cell lines (all available from Clontech Laboratories, Palo Alto, Calif., USA)—allow a wide host range to be infected with high efficiency; varying the multiplicity of infection readily adjusts the copy number of the integrated provirus. Retroviral vectors are available with a variety of selectable markers, such as resistance to neomycin, hygromycin, and puromycin, permitting ready selection of stable integrants.

The present invention further includes host cells comprising the vectors of the present invention, either present episomally within the cell or integrated, in whole or in part, into the host cell chromosome.

Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide GPCR-A-1 proteins with such post-translational modifications.

As noted earlier, host cells can be prokaryotic or eukaryotic. Representative examples of appropriate host cells include, but are not limited to, bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, murine ES cell lines (e.g., from strains 129/SV, C57/BL6, DBA-1, 129/SVJ), K562, Jurkat cells, and BW5147. Other mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

For example, phage lambda vectors will typically be packaged using a packaging extract (e.g., Gigapack® packaging extract, Stratagene, La Jolla, Calif., USA), and the packaged virus used to infect E. coli. Plasmid vectors will typically be introduced into chemically competent or electrocompetent bacterial cells.

E. coli cells can be rendered chemically competent by treatment, e.g., with CaCl₂, or a solution of Mg²⁺, Mn²⁺, Ca²⁺, Rb⁺ or K⁺, dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian Coli® XL10-Gold® Ultracompetent Cells (Stratagene, La Jolla, Calif., USA); DHSa competent cells (Clontech Laboratories, Palo Alto, Calif., USA); TOP10 Chemically Competent E. coli Kit (Invitrogen, Carlsbad, Calif., USA)).

Bacterial cells can be rendered electrocompetent—that is, competent to take up exogenous DNA by electroporation—by various pre-pulse treatments; vectors are introduced by electroporation followed by subsequent outgrowth in selected media. An extensive series of protocols is provided online in Electroprotocols (BioRad, Richmond, Calif., USA) (http://www.bio-rad.com/LifeScience/pdf/New_Gene_Pulser.pdf).

Vectors can be introduced into yeast cells by spheroplasting, treatment with lithium salts, electroporation, or protoplast fusion.

Spheroplasts are prepared by the action of hydrolytic enzymes—a snail-gut extract, usually denoted Glusulase, or Zymolyase, an enzyme from Arthrobacter luteus—to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol. For lithium-mediated transformation, yeast cells are treated with lithium acetate, which apparently permeabilizes the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6):339-46 (1989). For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194:182-7 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means.

For chemical transfection, DNA can be coprecipitated with CaPO₄ or introduced using liposomal and nonliposomal lipid-based agents. Commercial kits are available for CaPO₄ transfection (CalPhoS™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect™, Superfect® (Qiagen, Inc., Valencia, Calif., USA). Protocols for electroporating mammalian cells can be found online in Electroprotocols (Bio-Rad, Richmond, Calif., USA) (http://www.bio-rad.com/LifeScience/pdf/New_Gene_Pulser.pdf). See also, Norton et al. (eds.), Gene Transfer Methods: Introducing DNA into Living Cells and Organisms, BioTechniques Books, Eaton Publishing Co. (2000) (ISBN 1-881299-34-1), incorporated herein by reference in its entirety.

Other transfection techniques include transfection by particle embardment. See, e.g., Cheng et al., Proc. Natl. Acad. Sci. USA 90(10):4455-9 (1993); Yang et al., Proc. Natl. Acad. Sci. USA 87(24):9568-72 (1990).

Proteins

In another aspect, the present invention provides GPCR-A-1 proteins, various fragments thereof suitable for use as antigens (e.g., for epitope mapping) and for use as immunogens (e.g., for raising antibodies or as vaccines), fusions of GPCR-A-1 polypeptides and fragments to heterologous polypeptides, and conjugates of the proteins, fragments, and fusions of the present invention to other moieties (e.g., to carrier proteins, to fluorophores).

FIG. 3 presents the predicted amino acid sequences encoded by the GPCR-A-1 cDNA clone. The amino acid sequence is further presented, respectively, in SEQ ID NO: 3.

Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)- and the sequence determined from one individual of a species may differ from other allelic forms present within the population. Small deletions and insertions can often be found that do not alter the function of the protein.

Accordingly, it is an aspect of the present invention to provide proteins not only identical in sequence to those described with particularity herein, but also to provide isolated proteins at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program BLAST 2 SEQUENCES, available online at
http://www.ncbi.nlm.nih.gov/blast/bl2seq/bl2.html,
   To assess percent identity of amino acid sequences, the BLASTP module of BLAST 2 SEQUENCES is used with default values of (i) BLOSUM62 matrix, Henikoff et al., Proc. Natl. Acad. Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function.

Accordingly, it is an aspect of the present invention to provide proteins not only identical in sequence to those described with particularity herein, but also to provide isolated proteins having the sequence of GPCR-A-1 proteins, or portions thereof, with conservative amino acid substitutions. It is a further aspect to provide isolated proteins having the sequence of GPCR-A-1 proteins, and portions thereof, with moderately conservative amino acid substitutions. These conservatively-substituted and moderately conservatively-substituted variants can be naturally occurring or can result from human intervention.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

$$\begin{matrix} & A & R & N & D & C & Q & E & G & H & I & L & K & M & F & P & S & T & W & Y & V \\ A & 2 & - 1 & 0 & 0 & 0 & 0 & 0 & 0 & - 1 & - 1 & - 1 & 0 & - 1 & - 2 & 0 & 1 & 1 & - 4 & - 2 & 0 \\ R & - 1 & 5 & 0 & 0 & - 2 & 2 & 0 & - 1 & 1 & - 2 & - 2 & 3 & - 2 & - 3 & - 1 & 0 & 0 & - 2 & - 2 & - 2 \\ N & 0 & 0 & 4 & 2 & - 2 & 1 & 1 & 0 & 1 & - 3 & - 3 & 1 & - 2 & - 3 & - 1 & 1 & 0 & - 4 & - 1 & - 2 \\ D & 0 & 0 & 2 & 5 & - 3 & 1 & 3 & 0 & 0 & - 4 & - 4 & 0 & - 3 & - 4 & - 1 & 0 & 0 & - 5 & - 3 & - 3 \\ C & 0 & - 2 & - 2 & - 3 & 12 & - 2 & - 3 & - 2 & - 1 & - 1 & - 2 & - 3 & - 1 & - 1 & - 3 & 0 & 0 & - 1 & 0 & 0 \\ Q & 0 & \cdot 2 & 1 & 1 & - 2 & 3 & 2 & - 1 & 1 & - 2 & - 2 & 2 & - 1 & - 3 & 0 & 0 & 0 & - 3 & - 2 & - 2 \\ E & 0 & 0 & 1 & 3 & - 3 & 2 & 4 & - 1 & 0 & - 3 & - 3 & 1 & - 2 & - 4 & 0 & 0 & 0 & - 4 & - 3 & - 2 \\ G & 0 & - 1 & 0 & 0 & - 2 & - 1 & - 1 & 7 & - 1 & - 4 & - 4 & - 1 & - 4 & - 5 & - 2 & 0 & - 1 & - 4 & - 4 & - 3 \\ H & - 1 & 1 & 1 & 0 & - 1 & 1 & 0 & - 1 & 6 & - 2 & - 2 & 1 & - 1 & 0 & - 1 & 0 & 0 & - 1 & 2 & - 2 \\ I & - 1 & - 2 & - 3 & - 4 & - 1 & - 2 & - 3 & - 4 & - 2 & 4 & 3 & - 2 & 2 & 1 & - 3 & - 2 & - 1 & - 2 & - 1 & 3 \\ L & - 1 & - 2 & - 3 & - 4 & - 2 & - 2 & - 3 & - 4 & - 2 & 3 & 4 & - 2 & 3 & 2 & - 2 & - 2 & - 1 & - 1 & 0 & 2 \\ K & 0 & 3 & 1 & 0 & - 3 & 2 & 1 & - 1 & 1 & - 2 & - 2 & 3 & - 1 & - 3 & - 1 & 0 & 0 & - 4 & - 2 & - 2 \\ M & - 1 & - 2 & - 2 & - 3 & - 1 & - 1 & - 2 & - 4 & - 1 & 2 & 3 & - 1 & 4 & 2 & - 2 & - 1 & - 1 & - 1 & 0 & 2 \\ F & - 2 & - 3 & - 3 & - 4 & - 1 & - 3 & - 4 & - 5 & 0 & 1 & 2 & - 3 & 2 & 7 & - 4 & - 3 & - 2 & 4 & 5 & 0 \\ P & 0 & - 1 & - 1 & - 1 & - 3 & 0 & 0 & - 2 & - 1 & - 3 & - 2 & - 1 & - 2 & - 4 & 8 & 0 & 0 & - 5 & - 3 & - 2 \\ S & 1 & 0 & 1 & 0 & 0 & 0 & 0 & 0 & 0 & - 2 & - 2 & 0 & - 1 & - 3 & 0 & 2 & 2 & - 3 & - 2 & - 1 \\ T & 1 & 0 & 0 & 0 & 0 & 0 & 0 & - 1 & 0 & - 1 & - 1 & 0 & - 1 & - 2 & 0 & 2 & 2 & - 4 & - 2 & 0 \\ W & - 4 & - 2 & - 4 & - 5 & - 1 & - 3 & - 4 & - 4 & - 1 & - 2 & - 1 & - 4 & - 1 & 4 & - 5 & - 3 & - 4 & 14 & 4 & - 3 \\ Y & - 2 & - 2 & - 1 & - 3 & 0 & - 2 & - 3 & - 4 & 2 & - 1 & 0 & - 2 & 0 & 5 & - 3 & - 2 & - 2 & 4 & 8 & - 1 \\ V & 0 & - 2 & - 2 & - 3 & 0 & - 2 & - 2 & - 3 & - 2 & 3 & 2 & - 2 & 2 & 0 & - 2 & - 1 & 0 & - 3 & - 1 & 3\end{matrix}$$

For purposes herein, a “moderately conservative” replacement is any change having a nonnegative value in the PAM250 log-likelihood matrix reproduced herein above.

As is also well known in the art, relatedness of proteins can also be characterized using a functional test, the ability of the encoding nucleic acids to base-pair to one another at defined hybridization stringencies.

It is, therefore, another aspect of the invention to provide isolated proteins not only identical in sequence to those described with particularity herein, but also to provide isolated proteins (“hybridization related proteins”) that are encoded by nucleic acids that hybridize under high stringency conditions (as defined herein above) to all or to a portion of various of the isolated nucleic acids of the present invention (“reference nucleic acids”). It is a further aspect of the invention to provide isolated proteins (“hybridization related proteins”) that are encoded by nucleic acids that hybridize under moderate stringency conditions (as defined herein above) to all or to a portion of various of the isolated nucleic acids of the present invention (“reference nucleic acids”).

The hybridization related proteins can be alternative isoforms, homologues, paralogues, and orthologues of the GPCR-A-1 protein of the present invention. Particularly useful orthologues are those from other primate species, such as chimpanzee, rhesus macaque monkey, baboon, orangutan, and gorilla, from rodents, such as rats, mice, guinea pigs; from lagomorphs, such as rabbits, and from domestic livestock, such as cow, pig, sheep, horse, and goat.

Relatedness of proteins can also be characterized using a second functional test, the ability of a first protein competitively to inhibit the binding of a second protein to an antibody.

It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated GPCR-A-1 proteins of the present invention (“reference proteins”). Such competitive inhibition can readily be determined using immunoassays well known in the art.

Among the proteins of the present invention that differ in amino acid sequence from those described with particularity herein—including those that have deletions and insertions causing up to 10% non-identity, those having conservative or moderately conservative substitutions, hybridization related proteins, and cross-reactive proteins—those that substantially retain one or more GPCR-A-1 activities are particularly useful. As described above, those activities include G protein coupled receptor function in signal transduction, suppression of tumorigenesis and tumor metastasis.

Residues that are tolerant of change while retaining function can be identified by altering the protein at known residues using methods known in the art, such as alanine scanning mutagenesis, Cunningham et al., Science 244(4908):1081-5 (1989); transposon linker scanning mutagenesis, Chen et al., Gene 263(1-2):39-48 (2001); combinations of homolog- and alanine-scanning mutagenesis, Jin et al., J. Mol. Biol. 226(3):851-65 (1992); combinatorial alanine scanning, Weiss et al., Proc. Natl. Acad. Sci USA 97(16):8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, Epicentre Technologies Corporation, Madison, Wis., USA).

As further described below, the isolated proteins of the present invention can readily be used as specific immunogens to raise antibodies that specifically recognize GPCR-A-1 proteins, their isoforms, homologues, paralogues, and/or orthologues. The antibodies, in turn, can be used, inter alia, specifically to assay for the GPCR-A-1 proteins of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of GPCR-A-1 proteins, as for example by immunoprecipitation, and for use as specific agonists or antagonists of GPCR-A-1 action.

The isolated proteins of the present invention are also immediately available for use as specific standards in assays used to determine the concentration and/or amount specifically of the GPCR-A-1 proteins of the present invention. As is well known, ELISA kits for detection and quantitation of protein analytes typically include isolated and purified protein of known concentration for use as a measurement standard (e.g., the human interferon-γ OptEIA kit, catalog no. 555142, Pharmingen, San Diego, Calif., USA includes human recombinant gamma interferon, baculovirus produced).

The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al., Electrophoresis 21(6):1164-77 (2000), the disclosures of which are incorporated herein by reference in their entireties. Analogously, the isolated proteins of the present invention are also immediately available for use as specific biomolecule capture probes on BIACORE surface plasmon resonance probes. See Weinberger et al., Pharmacogenomics 1(4):395-416 (2000); Malmqvist, Biochem. Soc. Trans. 27(2):335-40 (1999).

The isolated proteins of the present invention are also useful as a therapeutic supplement in patients having a specific deficiency in GPCR-A-1 production.

In another aspect, the invention also provides fragments of various of the proteins of the present invention. The protein fragments are useful, inter alia, as antigenic and immunogenic fragments of GPCR-A-1.

By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least 8 contiguous amino acids, often at least 15 contiguous amino acids, have utility as immunogens for raising antibodies that recognize the proteins of the present invention. See, e.g., Lerner, “Tapping the immunological repertoire to produce antibodies of predetermined specificity,” Nature 299:592-596 (1982); Shinnick et al., “Synthetic peptide immunogens as vaccines,” Annu. Rev. Microbiol. 37:425-46 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20 amino acids in length, even 25 amino acids, 30 amino acids, 35 amino acids, or 50 amino acids or more in length. Of course, larger fragments having at least 75 amino acids, 100 amino acids, or even 150 amino acids are also useful, and at times preferred.

The present invention further provides fusions of each of the proteins and protein fragments of the present invention to heterologous polypeptides.

By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the GPCR-A-1 protein in altered arrangement; in such case, any of the GPCR-A-1 fragments can be considered heterologous to the other GPCR-A-1 fragments in the fusion protein. More typically, however, the heterologous polypeptide is not drawn from the GPCR-A-1 protein itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25 amino acids long, at least 50 amino acids long, and can be at least 75, 100, or even 150 amino acids long. Fusions that include the entirety of the proteins of the present invention have particular utility.

The heterologous polypeptide included within the fusion protein of the present invention is at least 6 amino acids in length, often at least 8 amino acids in length, and usefully at least 15, 20, and 25 amino acids in length. Fusions that include larger polypeptides, such as the IgG Fc region, and even entire proteins (such as GFP chromophore-containing proteins), have particular utility.

As described above in the description of vectors and expression vectors of the present invention, which discussion is incorporated herein by reference in its entirety, heterologous polypeptides to be included in the fusion proteins of the present invention can usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of GPCR-A-1 presence.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al., Yeast Hybrid Technologies, Eaton Publishing, (2000) (ISBN 1-881299-15-5); Fields et al., Trends Genet. 10(8):286-92 (1994); Mendelsohn et al., Curr. Opin. Biotechnol. 5(5):482-6 (1994); Luban et al., Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al., Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful protein fusions include those that permit display of the encoded protein on the surface of a phage or cell, fusions to intrinsically fluorescent proteins, such as green fluorescent protein (GFP), and fusions to the IgG Fc region, as described above, which discussion is incorporated here by reference in its entirety.

The proteins and protein fragments of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, normative bonds, and post-synthetic (post translational) modifications, either throughout the length of the protein or localized to one or more portions thereof.

As is well known in the art, when the isolated protein is used, e.g., for epitope mapping, the range of such nonnatural analogues, normative inter-residue bonds, or post-synthesis modifications will be limited to those that permit binding of the peptide to antibodies. When used as an immunogen for the preparation of antibodies in a non-human host, such as a mouse, the range of such nonnatural analogues, normative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Non-natural amino acids can be incorporated during solid phase chemical synthesis or by recombinant techniques, although the former is typically more common.

Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

For example, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide.

Biotin, for example (indirectly detectable through interaction with avidin, streptavidin, neutravidin, captavidin, or anti-biotin antibody), can be added using biotinoyl—(9-fluorenylmethoxycarbonyl)-L-lysine (FMOC biocytin) (Molecular Probes, Eugene, Oreg., USA). (Biotin can also be added enzymatically by incorporation into a fusion protein of a E. coli BirA substrate peptide.)

The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)—TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-S-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-b-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-?-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, all available from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl. Acad. Sci. USA 96(9):4780-5 (1999); Wang et al., Science 292(5516):498-500 (2001).

The isolated proteins, protein fragments and fusion proteins of the present invention can also include normative inter-residue bonds, including bonds that lead to circular and branched forms.

The isolated proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or O-linked glycosylation, the pattern of which will reflect both the availability of glycosylation sites on the protein sequence and the identity of the host cell. Further modification of glycosylation pattern can be performed enzymatically.

As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide.

Useful post-synthetic (and post-translational) modifications include conjugation to detectable labels, such as fluorophores.

A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents.

Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive.

Other labels that usefully can be conjugated to the proteins, protein fragments, and fusion proteins of the present invention include radioactive labels, echosonographic contrast reagents, and MRI contrast agents.

The proteins, protein fragments, and protein fusions of the present invention can also usefully be conjugated using cross-linking agents to carrier proteins, such as KLH, bovine thyroglobulin, and even bovine serum albumin (BSA), to increase immunogenicity for raising anti-GPCR-A-1 antibodies.

The proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

The isolated proteins of the present invention, including fusions thereof, can be produced by recombinant expression, typically using the expression vectors of the present invention as above-described or, if fewer than about 100 amino acids, by chemical synthesis (typically, solid phase synthesis), and, on occasion, by in vitro translation.

Production of the isolated proteins of the present invention can optionally be followed by purification. Purification of recombinantly expressed proteins is now well within the skill in the art. See, e.g., Thorner et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Expression and Protein Purification: Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Purification of chemically-synthesized peptides can readily be effected, e.g., by HPLC.

Accordingly, it is an aspect of the present invention to provide the isolated proteins of the present invention in pure or substantially pure form.

A purified protein of the present invention is an isolated protein, as above described, that is present at a concentration of at least 95%, as measured on a weight basis (w/w) with respect to total protein in a composition. Such purities can often be obtained during chemical synthesis without further purification, as, e.g., by HPLC. Purified proteins of the present invention can be present at a concentration (measured on a weight basis with respect to total protein in a composition) of 96%, 97%, 98%, and even 99%. The proteins of the present invention can even be present at levels of 99.5%, 99.6%, and even 99.7%, 99.8, or even 99.9% following purification, as by HPLC.

Although high levels of purity are particularly useful when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, in another aspect, the present invention provides the isolated proteins of the present invention in substantially purified form. A “substantially purified protein” of the present invention is an isolated protein, as above described, present at a concentration of at least 70%, measured on a weight basis with respect to total protein in a composition. Usefully, the substantially purified protein is present at a concentration, measured on a weight basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The proteins, fragments, and fusions of the present invention can usefully be attached to a substrate. The substrate can porous or solid, planar or non-planar; the bond can be covalent or noncovalent.

For example, the proteins, fragments, and fusions of the present invention can usefully be bound to a porous substrate, commonly a membrane, typically comprising nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention.

As another example, the proteins, fragments, and fusions of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in standard microtiter dish, the plastic is typically polystyrene.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween. The proteins, fragments, and fusions of the present invention can also be attached to a substrate suitable for use in surface plasmon resonance detection; so attached, the protein, fragment, or fusion of the present invention is useful for binding and then detecting secondary proteins that bind with sufficient affinity or avidity to the surface-bound protein to indicate biological interaction therebetween.

GPCR-A-1 Proteins

In a first series of protein embodiments, the invention provides an isolated GPCR-A-1 polypeptide having the amino acid sequence in SEQ ID NO: 3 which are full length GPCR-A-1 proteins. When used as immunogens, the full length proteins of the present invention can be used, inter alia, to elicit antibodies that bind to a variety of epitopes of the GPCR-A-1 protein.

The invention further provides fragments of the above-described polypeptides, particularly fragments having at least 6 amino acids, typically at least 8 amino acids, often at least 15 amino acids, and even the entirety of the sequence given in SEQ ID NO: 3.

The invention further provides fragments of at least 6 amino acids, typically at least 8 amino acids, often at least 15 amino acids, and even the entirety of the sequence given in SEQ ID NO: 6.

As described above, the invention further provides proteins that differ in sequence from those described with particularity in the above-referenced SEQ ID NOs., whether by way of insertion or deletion, by way of conservative or moderately conservative substitutions, as hybridization related proteins, or as cross-hybridizing proteins, with those that substantially retain a GPCR-A-1 activity particularly useful.

The invention further provides fusions of the proteins and protein fragments herein described to heterologous polypeptides.

Antibodies and Antibody-Producing Cells

In another aspect, the invention provides antibodies, including fragments and derivatives thereof, that bind specifically to GPCR-A-1 proteins and protein fragments of the present invention or to one or more of the proteins and protein fragments encoded by the isolated GPCR-A-1 nucleic acids of the present invention. The antibodies of the present invention can be specific for all of linear epitopes, discontinuous epitopes, or conformational epitopes of such proteins or protein fragments, either as present on the protein in its native conformation or, in some cases, as present on the proteins as denatured, as, e.g., by solubilization in SDS.

In other embodiments, the invention provides antibodies, including fragments and derivatives thereof, the binding of which can be competitively inhibited by one or more of the GPCR-A-1 proteins and protein fragments of the present invention, or by one or more of the proteins and protein fragments encoded by the isolated GPCR-A-1 nucleic acids of the present invention.

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to non-GPCR-A-1 proteins by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human brain, heart, colon, testis and uterus, as well as kidney, bone marrow, skeletal muscle and placenta.

Typically, the affinity or avidity of an antibody (or antibody multimer, as in the case of an IgM pentamer) of the present invention for a protein or protein fragment of the present invention will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of 8 or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties.

For example, peptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, Cold Spring Harbor Laboratory (1998) (ISBN: 0879693142); Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference, and often include multiple immunizations, either with or without adjuvants such as Freund's complete adjuvant and Freund's incomplete adjuvant.

Antibodies from nonhuman mammals can be polyclonal or monoclonal, with polyclonal antibodies having certain advantages in immunohistochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0-471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907); Howard et al. (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Techniques, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Briefly, however, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the proteins or protein fragments of the present invention can be cloned from hybridomas and thereafter expressed in other host cells. Nor need the two necessarily be performed together: e.g., genes encoding antibodies specific for the proteins and protein fragments of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant antibody production—either whole antibodies, antibody fragments, or antibody derivatives—can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established, Sidhu, Curr. Opin. Biotechnol. 11(6):610-6 (2000); Griffiths et al., Curr. Opin. Biotechnol. 9(1):102-8 (1998); Hoogenboom et al., Immunotechnology, 4(1):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the present invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(1):96-104 (1997); Edelman et al., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention can also be produced in plant cells, Giddings et al., Nature Biotechnol. 18(11):1151-5 (2000); Gavilondo et al., Biotechniques 29 (1):128-38 (2000); Fischer et al., J. Biol. Regul. Homeost. Agents 14(2):83-92 (2000); Fischer et al., Biotechnol. Appl. Biochem. 30 (Pt 2):113-6 (1999); Fischer et al., Biol. Chem. 380 (7-8):825-39 (1999); Russell, Curr. Top. Microbiol. Immunol. 240:119-38 (1999); and Ma et al., Plant Physiol. 109 (2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2):165-81 (1998), review and compare bacterial, yeast, insect and mammalian expression systems for expression of antibodies.

Antibodies of the present invention can also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998).

It is also an aspect of the present invention to provide antibody derivatives that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA. 81(21):6851-5 (1984); Sharon et al., Nature 309(5966):364-7 (1984); Takeda et al., Nature 314(6010):452-4 (1985), the disclosures of which are incorporated herein by reference in their entireties. Primatized and humanized antibodies typically include heavy and/or light chain CDRs from a murine antibody grafted into a non-human primate or human antibody V region framework, usually further comprising a human constant region, Riechmann et al., Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

The choice of label depends, in part, upon the desired use.

For example, when the antibodies of the present invention are used for immunohistochemical staining of tissue samples, the label can usefully be an enzyme that catalyzes production and local deposition of a detectable product.

Enzymes typically conjugated to antibodies to permit their immunohistochemical visualization are well known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 31,31-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cys, fluorescence resonance energy tandem fluorophores such as PerCP-Cy5.5, PE-Cys, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as ³³P, ³²P, ³⁵S, ³H, and ¹²⁵I.

As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²¹¹At, ²⁰³Pb, ¹⁹⁴Os, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y, ⁹⁰Sr, ⁸⁸Y, ⁷²Se, ⁶⁷Cu, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling

As would be understood, use of the labels described above is not restricted to the application as for which they were mentioned.

The antibodies of the present invention, including fragments and derivatives thereof, can also be conjugated to toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate, and it is, therefore, another aspect of the invention to provide antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention, attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

GPCR-A-1 Antibodies

In a first series of antibody embodiments, the invention provides antibodies, both polyclonal and monoclonal, and fragments and derivatives thereof, that bind specifically to a polypeptide having the amino acid sequence in SEQ ID NO: 3 which are full length GPCR-A-1 proteins.

Such antibodies are useful in in vitro immunoassays, such as ELISA, western blot or immunohistochemical assay of tumor tissues and cells. Such antibodies are also useful in isolating and purifying GPCR-A-1 proteins, including related cross-reactive proteins, by immunoprecipitation, immunoaffinity chromatography, or magnetic bead-mediated purification.

In another series of antibody embodiments, the invention provides antibodies, both polyclonal and monoclonal, and fragments and derivatives thereof, the specific binding of which can be competitively inhibited by the isolated proteins and polypeptides of the present invention.

In other embodiments, the invention further provides the above-described antibodies detectably labeled, and in yet other embodiments, provides the above-described antibodies attached to a substrate.

Pharmaceutical Compositions

GPCR-A-1 is important for signal transduction and tumor suppression; defects in GPCR-A-1 expression, activity, distribution, localization, and/or solubility are a cause of human disease, which disease can manifest as a disorder of brain, heart, colon, testis and uterus, as well as kidney, bone marrow, skeletal muscle and placenta function.

Accordingly, pharmaceutical compositions comprising nucleic acids, proteins, and antibodies of the present invention, as well as mimetics, agonists, antagonists, or inhibitors of GPCR-A-1 activity, can be administered as therapeutics for treatment of GPCR-A-1 defects.

Thus, in another aspect, the invention provides pharmaceutical compositions comprising the nucleic acids, nucleic acid fragments, proteins, protein fusions, protein fragments, antibodies, antibody derivatives, antibody fragments, mimetics, agonists, antagonists, and inhibitors of the present invention.

Such a composition typically contains from about 0.1 to 90% by weight of a therapeutic agent of the invention formulated in and/or with a pharmaceutically acceptable carrier or excipient.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties, and thus need not be described in detail herein.

Briefly, however, formulation of the pharmaceutical compositions of the present invention will depend upon the route chosen for administration. The pharmaceutical compositions utilized in this invention can be administered by various routes including both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

Oral dosage forms can be formulated as tablets, pills, dragees, capsules, liquids, gels, syrups, slurries, suspensions, and the like, for ingestion by the patient.

Solid formulations of the compositions for oral administration can contain suitable carriers or excipients, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid.

Agents that facilitate disintegration and/or solubilization can be added, such as the cross-linked polyvinyl pyrrolidone, agar, alginic acid, or a salt thereof, such as sodium alginate, microcrystalline cellulose, corn starch, sodium starch glycolate, and alginic acid.

Tablet binders that can be used include acacia, methylcellulose, sodium carboxymethylcellulose, polyvinylpyrrolidone (Povidone™), hydroxypropyl methylcellulose, sucrose, starch and ethylcellulose.

Lubricants that can be used include magnesium stearates, stearic acid, silicone fluid, talc, waxes, oils, and colloidal silica.

Fillers, agents that facilitate disintegration and/or solubilization, tablet binders and lubricants, including the aforementioned, can be used singly or in combination.

Solid oral dosage forms need not be uniform throughout.

For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Oral dosage forms of the present invention include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Additionally, dyestuffs or pigments can be added to the tablets or dragee coatings for product identification or to characterize the quantity of active compound, i.e., dosage.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone; and polyvinyl alcohol. The liquid formulations can also include solutions, emulsions, syrups and elixirs containing, together with the active compound(s), wetting agents, sweeteners, and coloring and flavoring agents.

The pharmaceutical compositions of the present invention can also be formulated for parenteral administration.

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution.

Intramuscular preparations, e.g. a sterile formulation of a suitable soluble salt form of the compounds of the present invention, can be dissolved and administered in a pharmaceutical excipient such as Water-for-Injection, 0.9% saline, or 5 glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Parenteral formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical compositions of the present invention can also be formulated to permit injectable, long-term, deposition.

The pharmaceutical compositions of the present invention can be administered topically.

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. In other transdermal formulations, typically in patch-delivered formulations, the pharmaceutically active compound is formulated with one or more skin penetrants, such as 2-N-methyl-pyrrolidone (NMP) or Azone.

Inhalation formulations can also readily be formulated. For inhalation, various powder and liquid formulations can be prepared.

The pharmaceutically active compound in the pharmaceutical compositions of the present inention can be provided as the salt of a variety of acids, including but not limited to hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art.

A “therapeutically effective dose” refers to that amount of active ingredient—for example GPCR-A-1 protein, fusion protein, or fragments thereof, antibodies specific for GPCR-A-1, agonists, antagonists or inhibitors of GPCR-A-1—which ameliorates the signs or symptoms of the disease or prevents progression thereof; as would be understood in the medical arts, cure, although desired, is not required.

The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial useful concentration range and route of administration.

For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are particularly useful.

The data obtained from cell culture assays and animal studies is used in formulating an initial dosage range for human use, and preferably provides a range of circulating concentrations that includes the ED50 with little or no toxicity. After administration, or between successive administrations, the circulating concentration of active agent varies within this range depending upon pharmacokinetic factors well known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions.

Therapeutic Methods

The present invention further provides methods of treating subjects having defects in GPCR-A-1—e.g., in expression, activity, distribution, localization, and/or solubility of GPCR-A-1—which can manifest as a disorder of brain, heart, colon, testis and uterus, as well as kidney, bone marrow, skeletal muscle and placenta function. As used herein, “treating” includes all medically-acceptable types of therapeutic intervention, including palliation and prophylaxis (prevention) of disease.

In one embodiment of the therapeutic methods of the present invention, a therapeutically effective amount of a pharmaceutical composition comprising GPCR-A-1 protein, fusion, fragment or derivative thereof is administered to a subject with a clinically-significant GPCR-A-1 defect.

Protein compositions are administered, for example, to complement a deficiency in native GPCR-A-1. In other embodiments, protein compositions are administered as a vaccine to elicit a humoral and/or cellular immune response to GPCR-A-1. The immune response can be used to modulate activity of GPCR-A-1 or, depending on the immunogen, to immunize against aberrant or aberrantly expressed forms, such as mutant or inappropriately expressed isoforms. In yet other embodiments, protein fusions having a toxic moiety are administered to ablate cells that aberrantly accumulate GPCR-A-1.

In another embodiment of the therapeutic methods of the present invention, a therapeutically effective amount of a pharmaceutical composition comprising nucleic acid of the present invention is administered. The nucleic acid can be delivered in a vector that drives expression of GPCR-A-1 protein, fusion, or fragment thereof, or without such vector.

Nucleic acid compositions that can drive expression of GPCR-A-1 are administered, for example, to complement a deficiency in native GPCR-A-1, or as DNA vaccines. Expression vectors derived from virus, replication deficient retroviruses, adenovirus, adeno-associated (AAV) virus, herpes virus, or vaccinia virus can be used—see, e.g., Cid-Arregui (ed.), Viral Vectors: Basic Science and Gene Therapy, Eaton Publishing Co., 2000 (ISBN: 188129935X)—as can plasmids.

Antisense nucleic acid compositions, or vectors that drive expression of GPCR-A-1 antisense nucleic acids, are administered to downregulate transcription and/or translation of GPCR-A-1 in circumstances in which excessive production, or production of aberrant protein, is the pathophysiologic basis of disease.

Antisense compositions useful in therapy can have sequence that is complementary to coding or to noncoding regions of the GPCR-A-1 gene. For example, oligonucleotides derived from the transcription initiation site, e.g., between positions −10 and +10 from the start site, are particularly useful.

Catalytic antisense compositions, such as ribozymes, that are capable of sequence-specific hybridization to GPCR-A-1 transcripts, are also useful in therapy. See, e.g., Phylactou, Adv. Drug Deliv. Rev. 44(2-3):97-108 (2000); Phylactou et al., Hum. Mol. Genet. 7(10):1649-53 (1998); Rossi, Ciba Found. Symp. 209:195-204 (1997); and Sigurdsson et al., Trends Biotechnol. 13(8):286-9 (1995), the disclosures of which are incorporated herein by reference in their entireties.

Other nucleic acids useful in the therapeutic methods of the present invention are those that are capable of triplex helix formation in or near the GPCR-A-1 genomic locus. Such triplexing oligonucleotides are able to inhibit transcription, Intody et al., Nucleic Acids Res. 28(21):4283-90 (2000); McGuffie et al., Cancer Res. 60(14):3790-9 (2000), the disclosures of which are incorporated herein by reference, and pharmaceutical compositions comprising such triplex forming oligos (TFOs) are administered in circumstances in which excessive production, or production of aberrant protein, is a pathophysiologic basis of disease.

In another embodiment of the therapeutic methods of the present invention, a therapeutically effective amount of a pharmaceutical composition comprising an antibody (including fragment or derivative thereof) of the present invention is administered. As is well known, antibody compositions are administered, for example, to antagonize activity of GPCR-A-1, or to target therapeutic agents to sites of GPCR-A-1 presence and/or accumulation.

In another embodiment of the therapeutic methods of the present invention, a pharmaceutical composition comprising a non-antibody antagonist of GPCR-A-1 is administered. Antagonists of GPCR-A-1 can be produced using methods generally known in the art. In particular, purified GPCR-A-1 can be used to screen libraries of pharmaceutical agents, often combinatorial libraries of small molecules, to identify those that specifically bind and antagonize at least one activity of GPCR-A-1.

In other embodiments a pharmaceutical composition comprising an agonist of GPCR-A-1 is administered. Agonists can be identified using methods analogous to those used to identify antagonists.

In still other therapeutic methods of the present invention, pharmaceutical compositions comprising host cells that express GPCR-A-1, fusions, or fragments thereof can be administered. In such cases, the cells are typically autologous, so as to circumvent xenogeneic or allotypic rejection, and are administered to complement defects in GPCR-A-1 production or activity.

In other embodiments, pharmaceutical compositions comprising the GPCR-A-1 proteins, nucleic acids, antibodies, antagonists, and agonists of the present invention can be administered in combination with other appropriate therapeutic agents. Selection of the appropriate agents for use in combination therapy can be made by one of ordinary skill in the art according to conventional pharmaceutical principles. The combination of therapeutic agents or approaches can act additively or synergistically to effect the treatment or prevention of the various disorders described above, providing greater therapeutic efficacy and/or permitting use of the pharmaceutical compositions of the present invention using lower dosages, reducing the potential for adverse side effects.

Transgenic Animals and Cells

In another aspect, the invention provides transgenic cells and non-human organisms comprising GPCR-A-1 isoform nucleic acids, and transgenic cells and non-human organisms with targeted disruption of the endogenous orthologue of the human GPCR-A-1 gene.

The cells can be embryonic stem cells or somatic cells. The transgenic non-human organisms can be chimeric, nonchimeric heterozygotes, and nonchimeric homozygotes.

Diagnostic Methods

The nucleic acids of the present invention can be used as nucleic acid probes to assess the levels of GPCR-A-1 mRNA in tumor tissues and cells, and antibodies of the present invention can be used to assess the expression levels of GPCR-A-1 proteins in tumor tissues and cells to diagnose cancer.

The following examples are offered for purpose of illustration, not limitation.

### EXAMPLE 1

### Identification and Characterization of cDNAs Encoding GPCR-A-1 Proteins

Bioinformatic algorithms were applied to human genomic sequence data to identify putative exons. Based on sequence information of one such exon, we identified a possible open reading frame. The predicted protein sequence from this potential exon shares significant homology with G protein coupled receptors.

Rapid Amplification of cDNA ends (RACE,

Clontech Laboratories, Palo Alto, Calif., USA) and direct PCR were used to obtain the coding region of the gene from human brain marathon-Ready™ cDNA (Clontech Labortory Inc). The final pair of primers used to amplify the coding region are: 34128F2 (5′-AGCGGGACTGGAGAGGAGTGAG-3′; SEQ ID NO: 1913) and 34128R2 (5′-CTAGGCATCCTTCCAAGTCAGGTGG-3′; SEQ ID NO: 1914). The PCR parameters were as follows: 94° C. 1 min; (94° C. 10 seconds; 64° C. 30 seconds; 72° C. 3 min.) for 35 cycles. The PCR reaction composition is as follows: 5 ul of cDNA; 5 ul of 10× amplification buffer; 1 ul dNTP (10 M); 3 ul of primer pairs; 1 ul of Taq polymerase Mix in 50 ul. The PCR product was cloned into a pGEM-Teasy vector and both strands of the DNA were sequenced using a MegaBACE™ automatic sequencer (Amersham Biosciences, Sunnyvale, Calif.). The clone spans 2.2 kilobases and appears to contain the entire coding region of the gene to which the exon contributes; for reasons described below, we termed this cDNA GPCR-A-1. Sequencing both strands provided us with the exact chemical structure of the cDNA, which is shown in FIG. 3 and further presented in the SEQUENCE LISTING as SEQ ID NO: 1, and placed us in actual physical possession of the entire set of single-base incremented fragments of the sequenced clone, starting at the 5′ and 3′ termini.

As shown in FIG. 3, the GPCR-A-1 cDNA spans 2225 nucleotides and contains an open reading frame from nucleotide 205 through and including nt 1431 (inclusive of termination codon), predicting a protein of 408 amino acids with a (posttranslationally unmodified) molecular weight of 45.3 kD. The clone appears full length, with the reading frame opening starting with a methionine and terminating with a stop codon.

BLAST query of genomic sequence identified four BACS, spanning over 192 kb, that constitute the minimum set of clones encompassing the cDNA sequence. Based upon the known origin of the BACs (GenBank accession numbers AL136478.9, Z95326.1, AL449196.1 and AL133379.3), the GPCR-A-1 gene can be mapped to human chromosome 6q22.1.

Comparison of the cDNA and genomic sequences identified eight exons. Exon organization is listed in Table 1. The genomic origin of the first three base pairs for the GPCR-A-1 cDNA is currently unknown.

TABLE 1
GPCR-A-1 Exon Structure

| Exon no. | cDNA range | genomic range | BAC accession |
|---|---|---|---|
| 1 | 4-377 | 38365-37992 | AL136478.9 |
| 2 | 378-553 | 24890-25065 | Z95326.1 |
| 3 | 554-681 | 20222-20349 | AL449196.1 |
| 4 | 682-841 | 93093-92934 | AL133379.3 |
| 5 | 842-998 | 84629-84473 | |
| 6 | 999-1051 | 82594-82542 | |
| 7 | 1052-1206 | 74904-74750 | |
| 8 | 1207-2225 | 46060-45042 | |

FIG. 2 schematizes the exon organization of the GPCR-A-1 clone.

At the top is shown the four bacterial artificial chromosomes (BACs), with GenBank accession numbers, that span the GPCR-A-1 locus.

As shown in FIG. 2, GPCR-A-1, encoding a protein of 408 amino acids, comprises exons 1-8. Predicted molecular weight, prior to any post-translational modification, is 45.3 kD. The cDNA clone appears full length, with the open reading frame starting with a methionine and terminating with a stop codon.

As further discussed in the examples herein, expression of GPCR-A-1 was assessed using RT-PCR. RT-PCR detected high level expression of GPCR-A-1 in brain, heart, colon, testis and uterus, as well as lower expression in kidney, bone marrow, skeletal muscle and placenta.

The sequence of the GPCR-A-1 cDNA was used as a BLAST query into the GenBank nr and dbEst databases. The nr database includes all non-redundant GenBank coding sequence translations, sequences derived from the 3-dimensional structures in the Brookhaven Protein Data Bank (PDB), sequences from SwissProt, sequences from the protein information resource (PIR), and sequences from protein research foundation (PRF). The dbEst (database of expressed sequence tags) includes ESTs, short, single pass read cDNA (mRNA) sequences, and cDNA sequences from differential display experiments and RACE experiments.

BLAST search identified multiple human ESTs, four mouse ESTs, one EST (BE030476.1) from pig, and one EST (BE104555.1) from rat as having sequence closely related to POSHL1.

Motif searches using Pfam (http://pfam.wustl.edu), SMART (http://smart.embl-heidelberg.de), and PROSITE pattern and profile databases (http://www.expasy.ch/prosite), identified several known domains shared with other membrane proteins and GPCRs.

FIG. 1 shows the domain structure of GPCR-A-1 and the alignmnet of the DUF6 domains with that of other proteins.

As schematized in FIG. 1, the newly isolated gene product shares certain protein domains and an overall structural organization with other G protein coupled receptors (GPCRs). The shared structural features strongly imply that GPCR-A-1 plays a role similar to that of other G protein coupled receptors in signal transduction, aberrant expression of which could lead to tumorigenesis and tumor metastasis.

Like other GPCRs, GPCR-A-1 has two DUF6 motifs (http://www.ncbi.nlm.nih.gov/Structure/cdd/wrpsb.cgi) and seven transmembrane domains (http://smart.embl-heidelberg.de/smart/show_{—}motifs.pl). The two DUF6 motifs in GPCR-A-1 cover amino acid sequences 81-200 and 229-354 of GPCR-A-1, respectively. DUF6 is a domain often found in integral membrane proteins.

Other signatures of the newly isolated GPCR-A-1 proteins were identified by searching the PROSITE database (http://www.expasy.ch/tools/scnpsitl.html). These include one N-glycosylation sites (313-316), one cAMP- and cGMP-dependent protein kinase phosphorylation site (305-308), two protein kinase C phosphorylation sites (43-45 and 80-82), three Casein kinase II phosphorylation sites (24-27, 245-248, and 399-402), and multiple N-myristoylation sites.

Possession of the genomic sequence permitted search for promoter and other control sequences for the GPCR-A-1 gene.

A putative transcriptional control region, inclusive of promoter and downstream elements, was defined as 1 kb around the transcription start site, itself defined as the first nucleotide of the GPCR-A-1 cDNA clone. The region, drawn from sequence of BAC AL136478.9, has the sequence given in SEQ ID NO: 24, which lists 1000 nucleotides before the first exon.

Transcription factor binding sites were identified using a web based program (http://motif.genome.ad.jp/), including binding sites for MyoD (379-388), CdxA (315-321), STATx (260-268), c-Myb (789-798, with numbering according to SEQ ID NO: 24), amongst others.

We have thus identified a newly described human gene, GPCR-A-1, which shares certain protein domains and an overall structural organization with other GPCRs. The shared structural features strongly imply that the GPCR-A-1 protein plays a role similar to other GPCRs, in signal transduction, making the GPCR-A-1 proteins and nucleic acids clinically useful diagnostic markers and potential therapeutic agents for cancer.

### EXAMPLE 2

### Preparation and Labeling of Useful Fragments of GPCR-A-1

Useful fragments of GPCR-A-1 are produced by PCR, using standard techniques, or solid phase chemical synthesis using an automated nucleic acid synthesizer. Each fragment is sequenced, confirming the exact chemical structure thereof.

The exact chemical structure of preferred fragments is provided in the attached SEQUENCE LISTING, the disclosure of which is incorporated herein by reference in its entirety. The following summary identifies the fragments whose structures are more fully described in the SEQUENCE LISTING:

SEQ ID NO: 1 (nt, full length GPCR-A-1 cDNA)

SEQ ID NO: 2 (nt, cDNA ORF)

SEQ ID NO: 3 (aa, full length GPCR-A-1 protein)

SEQ ID NO: 4 (nt, (nt 107-159) portion of GPCR-A-1)

SEQ ID NO: 5 (nt, (nt 730-1156) portion of GPCR-A-1)

SEQ ID NO: 6 (aa, (aa 176-317) CDS entirely within SEQ ID NO: 5)

SEQ ID NO: 7 (nt, (nt 1571-2094) portion of GPCR-A-1)

SEQ ID NOs: 8-15 (nt, exons 1-8 (from genomic sequence))

SEQ ID NOs: 16-23 (nt, 500 bp genomic amplicon centered about exons 1-8)

SEQ ID NO: 24 (nt, 1000 bp putative promoter)

SEQ ID NOs: 25-61 (nt, 17-mers scanning (nt 107-159) portion of GPCR-A-1)

SEQ ID NOs: 62-90 (nt, 25-mers scanning (nt 107-159) portion of GPCR-A-1)

SEQ ID NOs: 91-501 (nt, 17-mers scanning (nt 730-1156) portion of GPCR-A-1)

SEQ ID NOs: 502-904 (nt, 25-mers scanning (nt 730-1156) portion of GPCR-A-1)

SEQ ID NOs: 905-1412 (nt, 17-mers scanning (nt 1571-2094) portion of GPCR-A-1)

SEQ ID NOs: 1413-1912 (nt, 25-mers scanning (nt 1571-2094) portion of GPCR-A-1)

SEQ ID NO: 1913 (nt, primer 34128F2 for amplification of the GPCR-A-1 coding region)

SEQ ID NO: 1914 (nt, primer 34128R2 for amplification of the GPCR-A-1 coding region)

SEQ ID NO: 1915 (nt, primer 34128F4 for expression analysis of GPCR-A-1 by RT-PCR)

SEQ ID NO: 1916 (nt, primer 34128R3 for expression analysis of GPCR-A-1 by RT-PCR)

SEQ ID NO: 1917 (aa, consensus sequence of the DUF6 motif 1)

SEQ ID NO: 1918 (aa, sequence of the GPCR-A-1 DUF6 motif 1)

SEQ ID NO: 1919 (aa, sequence of the DUF6 motif 1 of protein gi: 6136579)

SEQ ID NO: 1920 (aa, sequence of the DUF6 motif 1 of protein gi: 3128363)

SEQ ID NO: 1921 (aa, sequence of the DUF6 motif 1 of protein gi: 6136689)

SEQ ID NO: 1922 (aa, consensus sequence of the DUF6 motif 2)

SEQ ID NO: 1923 (aa, sequence of the GPCR-A-1 DUF6 motif 2)

SEQ ID NO: 1924 (aa, sequence of the DUF6 motif 2 of protein gi: 6136694)

SEQ ID NO: 1925 (aa, sequence of the DUF6 motif 2 of protein gi: 3256456)

SEQ ID NO: 1926 (aa, sequence of the DUF6 motif 2 of protein gi: 3257364)

Upon confirmation of the exact structure, each of the above-described nucleic acids of confirmed structure is recognized to be immediately useful as a GPCR-A-1-specific probe.

For use as labeled nucleic acid probes, the above-described GPCR-A-1 nucleic acids are separately labeled by random priming. As is well known in the art of molecular biology, random priming places the investigator in possession of a near-complete set of labeled fragments of the template of varying length and varying starting nucleotide.

The labeled probes are used to identify the GPCR-A-1 gene on a Southern blot, and are used to measure expression of GPCR-A-1 mRNA on a northern blot and by RT-PCR, using standard techniques.

### EXAMPLE 3

### Expression Analysis of GPCR-A-1 by RT-PCR

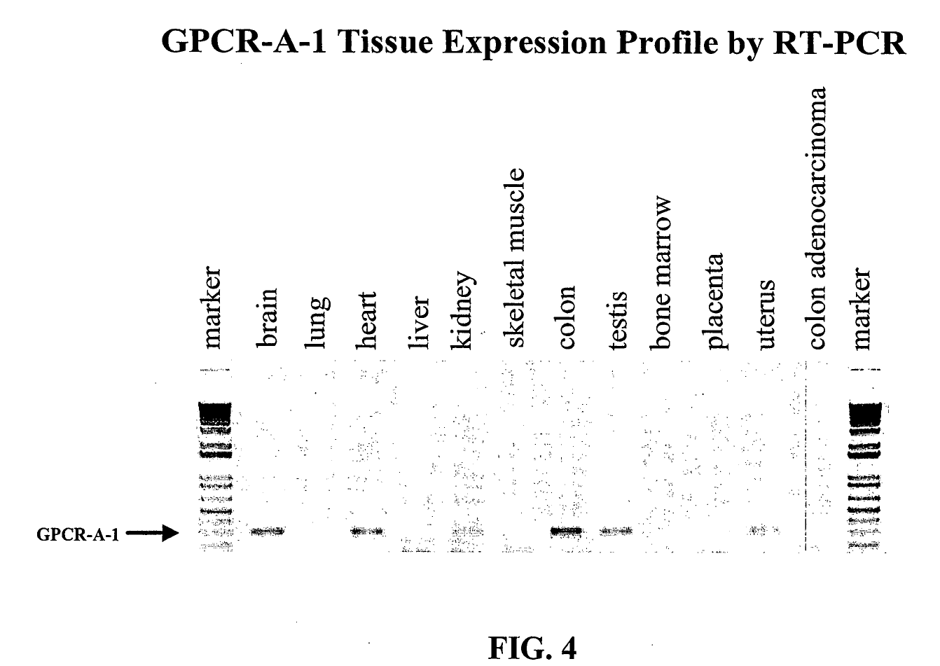

The Advantage 2 PCR amplification kit and PCR cDNAs of different human tissues were obtained from Clontech Laboratories Inc. (Palo Alto, Calif.). The PCR parameters were set-up as follows, 94° C. 10 seconds; 64° C. 30 seconds; 720C 40 seconds for 36 cycles. The PCR composition was as follows: 0.5 ul of cDNA; 2.5 ul of 10× amplification buffer; 0.5 ul dNTP (10 M); 1 ul of primer pairs (10 M each; 34128F4 (5′-CCACCTGACTTGGAAGGATGCCTAG-3′; SEQ ID NO: 1915) and 34128R3 (5′-GAGCTCCTGTGGCATGCATCACTC-3′; SEQ ID NO: 1916)); 0.5 ul of Advantage polymerase Mix in 25 ul reaction mixture. The amplified DNA products were resolved in 1.2% agarose gel in TAE buffer. The gel was scanned using Typhoon™ Imaging System (Amersham Biosciences, Sunnyvale, Calif.). RT-PCR detected high level expression of GPCR-A-1 in brain, heart, colon, testis and uterus, as well as lower expression in kidney, bone marrow, skeletal muscle and placenta. No expression was detected in liver, lung, as well as colon adenocarcinoma (FIG. 4).

### EXAMPLE 4

### Production of GPCR-A-1 Protein

The full length GPCR-A-1 cDNA clone is cloned into the mammalian expression vector pcDNA3.1/HISA (Invitrogen, Carlsbad, Calif., USA), transfected into COS7 cells, transfectants selected with G418, and protein expression in transfectants confirmed by detection of the anti-Xpress™ epitope according to manufacturer's instructions. Protein is purified using immobilized metal affinity chromatography and vector-encoded protein sequence is then removed with enterokinase, per manufacturer's instructions, followed by gel filtration and/or HPLC.

Following epitope tag removal, GPCR-A-1 protein is present at a concentration of at least 70w, measured on a weight basis with respect to total protein (i.e., w/w), and is free of acrylamide monomers, bis acrylamide monomers, polyacrylamide and ampholytes. Further HPLC purification provides GPCR-A-1 protein at a concentration of at least 95%, measured on a weight basis with respect to total protein (i.e., w/w).

### EXAMPLE 5

### Production of Anti-GPCR-A-1 Antibody

Purified proteins prepared as in Example 4 are conjugated to carrier proteins and used to prepare murine monoclonal antibodies by standard techniques. Initial screening with the unconjugated purified proteins, followed by competitive inhibition screening using peptide fragments of the GPCR-A-1, identifies monoclonal antibodies with specificity for GPCR-A-1.

### EXAMPLE 6

### Use of GPCR-A-1 Probes and Antibodies for Diagnosis of Cancer

After informed consent is obtained, samples are drawn from tumor tissue or cells and tested for GPCR-A-1 mRNA levels by standard techniques and tested additionally for GPCR-A-1 protein levels using anti-GPCR-A-1 antibodies in a standard ELISA.

### EXAMPLE 7

### Use of GPCR-A-1 Nucleic Acids, Proteins, and Antibodies in Therapy

Once over-expression of GPCR-A-1 is detected in patients, GPCR-A-1 antisense RNA or GPCR-A-1-specific antibody is introduced into disease cells to reduce the amount of the protein.

Once mutations of GPCR-A-1 have been detected in patients, normal GPCR-A-1 is reintroduced into the patient's disease cells by introduction of expression vectors that drive GPCR-A-1 expression or by introducing GPCR-A-1 proteins into cells. Antibodies for the mutated forms of GPCR-A-1 are used to block the function of the abnormal forms of the protein.

### EXAMPLE 8

### GPCR-A-1 Disease Associations

Diseases that map to the GPCR-A-1 chromosomal by OMIM: http://www.ncbi.nlm.nih.gov/entrez/query.fcgi) are shown in Table 2:

TABLE 2
Diseases mapped to human chromosome 6q22.1.

| | | chromosomal |
|---|---|---|
| mim_num | disease | location |
| 161950 | IgA NEPHROPATHY | 6g22-q23 |
| 164200 | OCULODENTODIGITAL DYSPLASIA | 6q22-q24 |
| 601228 | HEREDITARY MIXED POLYPOSIS | 6q |
| | SYNDROME | |
| 603175 | SCHIZOPHRENIA 5 | 6q13-q26 |
| 604201 | HEPATIC FIBROSIS, SEVERE, | 6q22-q23 |
| | SUSCEPTIBILITY TO, DUE TO | |
| | SCHISTOSOMA MANSONI INFECTION | |
| 600089 | PANCREATIC BETA CELL AGENESIS | Chr.6 |
| | WITH NEONATAL DIABETES | |
| | MELLITUS | |

One of these diseases, the hereditary mixed polyposis syndrome, has physiological characteristics consistent with alteration of a GPCR-A-1-like gene. The hereditary mixed polyposis syndrome is characterized by atypical juvenile polyps, colonic adenomas, and colorectal carcinomas. Most older individuals presented with colorectal carcinoma; younger individuals, most of whom had undergone screening by colonoscopy, tended to present with benign colonic lesions, often the characteristic polyp of the hereditary mixed polyposis syndrome.

Two hot spots were found on chromosome 6q that may harbor putative tumor suppressor genes involved not only in the oncogenesis but maybe also in the malignant and metastatic progression of sporadic endocrine pancreatic tumors (EPTs). A loss of heterozygosity (LOH) in a majority of patients was seen at 6q22.1 (D6S262) and 6q23-q24 (D6S310-UTRN) with LOH-frequencies of 50.0% and 41.2 to 56.3%, respectively. Barghorn et al., Am. J. Pathol. 158:1903-1911 (2001). Abnormality of chromosome 6q22 is also associated with several other cancers, such as parathyroid carcinomas (Kytosa et al., Am. J. Pathol. 157:579-586 (2000)), as well as bladder cancer progression (Richter et al., Cancer Res. 59:5687-5691 (1999)).

Mutations of GPCR-A-1 may result in one of the syndromes listed above, or even another disease not listed here.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. An isolated nucleic acid that encodes a G protein coupled receptor, mutations of which cause cancer, comprising:
(a) a nucleotide sequence selected from the group consisting of:
(i) SEQ ID NO: 1;
(ii) the complete complement of the sequences set forth in (i);
(iii) the nucleotide sequence of SEQ ID NO: 2;
(iv) a degenerate variant of the sequences set forth in (iii); and
(v) the complete complement of the sequences set forth in (iii) and (iv); or
(b) a nucleotide sequence selected from the group consisting of:
(i) a nucleotide sequence that encodes a polypeptide having the sequence of SEQ ID NO: 3;
(ii) a nucleotide sequence that encodes a polypeptide having the sequence of SEQ ID NO: 3, with conservative amino acid substitutions; and
(iii) the complete complement of the sequences set forth in (i) and (ii), wherein said isolated nucleic acid comprising a nucleotide sequence selected from group (b) is no more than about 100 kb in length.

2. The isolated nucleic acid of claim 1, wherein said nucleic acid, or the complement of said nucleic acid, encodes a polypeptide having G protein coupled receptor activity.

3. The isolated nucleic acid of claim 1, wherein said nucleic acid, or the complement of said nucleic acid, is expressed in brain, heart, colon, testis and uterus, as well as in kidney, bone marrow, skeletal muscle and placenta.

4. A nucleic acid probe, comprising:
the nucleic acid of claim 1,
wherein said probe is no longer than about 100 kb in length.

5. The probe of claim 4, wherein said probe is detectably labeled.

6. The probe of claim 4, attached to a substrate.

7. A microarray, wherein at least one probe of said array is a probe of claim 4.

8. The isolated nucleic acid molecule of claim 1, wherein said nucleic acid molecule is operably linked to one or more expression control elements.

9. A replicable vector comprising a nucleic acid molecule of claim 1.

10. A replicable vector comprising an isolated nucleic acid molecule of claim 8.

11. A host cell transformed to contain the nucleic acid molecule of claim 1, or the progeny thereof.

12. A method for producing a polypeptide, the method comprising:
culturing the host cell of claim 11 under conditions in which the protein encoded by said nucleic acid molecule is expressed.

13. An isolated polypeptide produced by the method of claim 12.

14. An isolated polypeptide, comprising:
(a) an amino acid sequence having the sequence listed in SEQ ID NO: 3;
(b) an amino acid sequence having at least 65% amino acid sequence identity to that of (a); or
(c) an amino acid sequence according to (a) in which at least 95% of deviations from the sequence of (a) are conservative substitutions.

15. A fusion protein comprising a polypeptide of claim 14 fused to a heterologous amino acid sequence.

16. The fusion protein of claim 15, wherein said heterologous amino acid sequence is a detectable moiety.

17. The fusion protein of claim 16, wherein said detectable moiety is fluorescent.

18. The fusion protein of claim 15, wherein said heterologous amino acid sequence is an Ig Fc region.

19. An isolated antibody, or antigen-binding fragment or derivative thereof, the binding of which can be competitively inhibited by a polypeptide of claim 14.

20. A transgenic non-human animal modified to contain the nucleic acid molecule of claim 1.

21. A transgenic non-human animal unable to express the endogenous orthologue of the nucleic acid molecule of claim 1.

22. A method of identifying agents that modulate the expression of GPCR-A-1, the method comprising:
contacting a cell or tissue sample believed to express GPCR-A-1 with a chemical or biological agent, and then
comparing the amount of GPCR-A-1 expression in said cell or tissue sample with that of a control,
changes in the amount relative to control identifying an agent that modulates expression of GPCR-A-1.

23. A method of identifying agonists and antagonists of GPCR-A-1, the method comprising:
contacting a cell or tissue sample believed to express GPCR-A-1 with a chemical or biological agent, and then
comparing the activity of GPCR-A-1 with that of a control, increased activity relative to a control identifying an agonist, decreased activity relative to a control identifying an antagonist.

24. A purified agonist of the polypeptide of claim 14.

25. A purified antagonist of the polypeptide of claim 14.

26. A method of identifying a specific binding partner for a polypeptide according to claim 14, the method comprising:
contacting said polypeptide to a potential binding partner; and
determining if the potential binding partner binds to said polypeptide.

27. The method of claim 26, wherein said contacting is performed in vivo.

28. A purified binding partner of the polypeptide of claim 14.

29. A method for detecting a target nucleic acid in a sample, said target being a nucleic acid according to claim 1, the method comprising:
(a) hybridizing the sample with a probe comprising at least 17 contiguous nucleotides of a sequence complementary to said target nucleic acid in said sample under high stringency hybridization conditions, and
(b) detecting the presence or absence, and optionally the amount, of said binding.

30. A method of diagnosing a disease caused by mutation in GPCR-A-1, comprising:
detecting said mutation in a sample of nucleic acids that derives from a subject suspected to have said disease.

31. A method of diagnosing or monitoring a disease caused by altered expression of GPCR-A-1, comprising:
determining the level of expression of GPCR-A-1 in a sample of nucleic acids or proteins that derives from a subject suspected to have said disease,
alterations from a normal level of expression providing diagnostic and/or monitoring information.

32. A diagnostic composition comprising the nucleic acid of claim 1, said nucleic acid being detectably labeled.

33. The diagnostic composition of claim 32, wherein said composition is further suitable for in vivo administration.

34. A diagnostic composition comprising the polypeptide of claim 14, said polypeptide being detectably labeled.

35. The diagnostic composition of claim 34, wherein said composition is further suitable for in vivo administration.

36. A diagnostic composition comprising the antibody, or antigen-binding fragment or derivative thereof, of claim 19.

37. The diagnostic composition of claim 36, wherein said antibody or antigen-binding fragment or derivative thereof is detectably labeled.

38. The diagnostic composition of claim 37, wherein said composition is further suitable for in vivo administration.

39. A pharmaceutical composition comprising the nucleic acid of claim 1 and a pharmaceutically acceptable excipient.

40. A pharmaceutical composition comprising the polypeptide of claim 14 and a pharmaceutically acceptable excipient.

41. A pharmaceutical composition comprising the antibody or antigen-binding fragment or derivative thereof of claim 19 and a pharmaceutically acceptable excipient.

42. A pharmaceutical composition comprising the agonist of claim 24 and a pharmaceutically acceptable excipient.

43. A pharmaceutical composition comprising the antagonist of claim 25 and a pharmaceutically acceptable excipient.

44. A method for treating or preventing a disorder associated with decreased expression or activity of GPCR-A-1, the method comprising administering to a subject in need of such treatment an effective amount of the pharmaceutical composition of claim 39.

45. A method for treating or preventing a disorder associated with increased expression or activity of GPCR-A-1, the method comprising administering to a subject in need of such treatment an effective amount of the pharmaceutical composition of claim 41.

46. A method of modulating the expression of a nucleic acid according to claim 1, the method comprising:
administering an effective amount of an agent which modulates the expression of a nucleic acid according to claim 1.

47. A method of modulating at least one activity of a polypeptide according to claim 14, the method comprising:
administering an effective amount of an agent which modulates at least one activity of a polypeptide according to claim 14.
